(19) United States
(12) Patent Application Publication
(10) Pub. No.: US 2023/0233608 A1
(43) Pub. Date: Jul. 27, 2023

(54) REGULATED BIOCIRCUIT SYSTEMS

(71) Applicant: OBSIDIAN THERAPEUTICS, INC., Cambridge, MA (US)

(72) Inventors: Peter Barrett, Cambridge, MA (US); Michael N. Gladstone, Cambridge, MA (US); Tariq A. Kassum, Cambridge, MA (US); Vipin Suri, Belmont, MA (US); Dan Jun Li, Cambridge, MA (US); Dexue Sun, Cambridge, MA (US); Brian Dolinski, Cambridge, MA (US)

(73) Assignee: OBSIDIAN THERAPEUTICS, INC., Cambridge, MA (US)

(21) Appl. No.: 17/888,808

(22) Filed: Aug. 16, 2022

Related U.S. Application Data
(63) Continuation-in-part of application No. 16/092,829, filed on Oct. 11, 2018, now Pat. No. 11,446,398.
(60) Provisional application No. 62/466,596, filed on Mar. 3, 2017.
(60) Provisional application No. 62/320,864, filed on Apr. 11, 2016.

(51) Int. Cl. A61K 35/17; C07K 14/54

(52) U.S. Cl. CPC A61K 35/17; C07K 14/5443; C07K 14/5434

(57) ABSTRACT

The present invention provides regulatable biocircuit systems. Such systems provide modular and tunable protein expression systems in support of the discovery and development of therapeutic modalities.

### CROSS REFERENCE TO RELATED APPLICATIONS

This application is a continuation-in-part of U.S. patent application Ser. No. 16/092,829, filed Oct. 11, 2018, which is a U.S. national stage application under 35 USC § 371 of PCT/US2017/026950, filed Apr. 11, 2017, which claims priority to U.S. Provisional Patent Application No. 62/320,864, filed Apr. 11, 2016, entitled Regulated Biocircuit Systems, and U.S. Provisional Patent Application No. 62/466,596, filed Mar. 3, 2017, entitled Regulated Biocircuit Systems, the contents each of which are herein incorporated by reference in their entireties.

### SEQUENCE LISTING

The instant application contains a Sequence Listing in XML format. The Sequence Listing, named 1306615.xml was created on Feb. 13, 2023, is 60.6 Kilobytes in size, and is hereby incorporated by reference in its entirety.

### FIELD OF THE INVENTION

The invention relates to regulatable and tunable biocircuit systems for the development of controlled and/or regulated therapeutic systems, e.g., biocircuits.

### BACKGROUND OF THE INVENTION

Gene therapy is revolutionizing medicine and offering new promise for the treatment of previously intractable conditions. However, current technologies do not allow titration of the timing or levels of target protein induction. This has rendered many potential gene therapy applications difficult or impossible to safely and effectively deploy.

Inadequate exogenous and/or endogenous gene control is a critical issue in numerous gene therapy settings. This lack of tunability also makes it difficult to safely express proteins with narrow or uncertain therapeutic windows or those requiring more titrated or transient expression.

One approach to regulated protein expression or function is the use of Destabilizing Domains (DDs). Destabilizing domains are small protein domains that can be appended to a target protein of interest. DDs render the attached protein of interest unstable in the absence of a DD-binding ligand such that the protein is rapidly degraded by the ubiquitin-proteasome system of the cell (Stankunas, K., et al., (2003). Mol. Cell 12, 1615-1624; Banaszynski, et al., (2006) Cell; 126(5): 995-1004; reviewed in Banaszynski, L. A., and Wandless, T. J. (2006) Chem. Biol.; 13, 11-21 and Rakhit R, Navarro R, Wandless T J (2014) Chem Biol. September 18; 21(9):1238-52). However, when a specific small molecule ligand binds its intended DD as a ligand binding partner, the instability is reversed and protein function is restored. Such a system is herein referred to as a biocircuit, with the canonical DD-containing biocircuit described above being the prototypical model biocircuit.

It is believed that improvements of biocircuits, including those containing DDs can form the basis of a new class of cell and gene therapies that employ tunable and temporal control of gene expression and function. Such novel moieties are described by the present inventors as stimulus response elements (SREs) which act in the context of an effector module to complete a biocircuit arising from a stimulus and ultimately producing a signal or outcome. When properly formatted with a polypeptide payload, and when activated by a particular stimulus, e.g., a small molecule, biocircuit systems can be used to regulate transgene and/or protein levels either up or down by perpetuating a stabilizing signal or destabilizing signal. This approach has many advantages over existing methods of regulating protein function and/or expression, which are currently focused on top level transcriptional regulation via inducible promoters.

Beyond the initial work on destabilizing domains (Banaszynski, et al., (2006) Cell; 126(5): 995-1004; U.S. Pat. Nos. 8,173,792 and 8,530,636, the contents of which are each incorporated herein by reference in their entirety), is the development of expanded biocircuit systems such as those taught in the present application including stimulus response elements (SREs) which go far beyond the destabilizing or dimerization domains of the art. Such therapies represent a significant improvement on existing gene therapy strategies, and could also expand the universe of protein therapeutics that can be safely and effectively incorporated into gene therapy modalities, including applications that have previously been considered unsuitable for therapeutic use.

### SUMMARY OF THE INVENTION

The invention provides biocircuit systems which may comprise an effector module responsive to at least one stimulus. The effector module may comprise a first component and a second component which may be independently selected from, but are not limited to, a peptide, peptide complex, peptide-protein complex, protein, fusion protein, protein complex, protein-protein complex. In one aspect, the biocircuit system may comprise two or more effector modules.

In one aspect, the first component may be a stimulus response element (SRE) comprising one or more regions derived from a payload, one or more regions derived from a Target of a ligand binding partner pair, one or more regions derived from an antibody, and/or an SRE.

In one aspect, the second component may be a payload construct comprising a payload, a Target of a ligand binding partner pair, and/or an antibody or a functional fragment thereof.

In one aspect, the first component may be a stimulus response element (SRE) comprising one or more regions derived from a payload, the payload may have a sequence such as, but not limited to, one or more regions derived from a Target of a ligand binding partner pair listed in Table 2 or Table 3 of U.S. Patent Application Publication No. 20190192691, one or more regions derived from an antibody listed in Table 5 of U.S. Patent Application Publication No. 20190192691, and/or an SRE listed in Table 4 of U.S. Patent Application Publication No. 20190192691. Each of Tables 2-4 is hereby incorporated by reference herein in its entirety.

In one aspect, the second component may be a payload construct comprising a payload, the payload may have a sequence such as, but not limited to, a Target of a ligand binding partner pair listed in Table 2 of U.S. Patent Application Publication No. 20190192691, Table 3 of U.S. Patent Application Publication No. 20190192691 or functional fragment thereof, and/or an antibody listed in Table 5 of U.S. Patent Application Publication No. 20190192691 or a functional fragment thereof. Table 5 is hereby incorporated by reference herein in its entirety.

In one aspect, at least one of the first or second component of the effector module may comprise one or more post translational modifications such as, but not limited to, acetylation, phosphorylation, ubiquitination, carboxylation, deamination, deamination, deacetylation, dihydroxylation, dephosphorylation, formylation, gamma-carboxyglutamation, glutathionylation, glycation, hydroxylation, methylation, nitration, sumoylation, N- or O-transglutamination, glycosylation and farnesylation.

In one aspect, the biocircuit system may be, but is not limited to, a DD biocircuit system, a Dimer biocircuit system, a CAR biocircuit system, a Receptor biocircuit system, and a Cell biocircuit system.

In one aspect, the effector module of the biocircuit system may comprise a signal sequence, a cleavage and/or processing feature, a targeting and/or penetrating peptide, and/or a linker.

In one aspect, the effector module of the biocircuit system may comprise a signal sequence selected from those listed in Table 6 of U.S. Patent Application Publication No. 20190192691, a cleavage and/or processing feature selected from those listed in Table 7 of U.S. Patent Application Publication No. 20190192691, a targeting and/or penetrating peptide selected from those listed in Tables 8 or 10 of U.S. Patent Application Publication No. 20190192691, and/or a linker selected from those listed in Tables 9, 11 and 12 U.S. Patent Application Publication No. 20190192691. Tables 6-12 are hereby incorporated by reference herein in their entireties.

In one aspect, the stimulus of the biocircuit system may be, but is not limited to, a ligand, an externally added or endogenous metabolite, the presence or absence of a defined ligand, pH, temperature, light, ionic strength, cellular location, subject site, microenvironment, the presence or concentration of one or more cations or one or more anions, an effector module, a concentration gradient of ions, biomolecules or the like, and the presence or concentration of one or more metal ions.

In another aspect, the stimulus of the biocircuit system may be a ligand. The ligand may be, but is not limited to, any of the ligands of Tables 1-3 of U.S. Patent Application Publication No. 20190192691, a protein, peptide, nucleic acid, lipid, lipid derivative, sterol, steroid, metabolite, metabolite derivative, and small molecule. Tables 1-3 are incorporated by reference herein in their entireties. The ligand may be complexed or bound to another molecule.

In one aspect, the stimulus is a ligand which is a small molecule. As a non-limiting example, the small molecule may be cell permeable.

In one aspect, the stimulus of the biocircuit system may be a cellular location such as, but not limited to, the nucleus, the cytoplasm, a membrane, lysosome, mitochondria, endoplasmic reticulum, cellular organelle, cytoskeletal protein or subregion, intracellular membrane surface, a transmembrane region, and the extracellular matrix.

In one aspect, the stimulus of the biocircuit system may be a subject site such as, but not limited to, a location in the subject selected from the blood, plasma, an organ selected from liver, kidney, brain, heart, lung, bone, and bone marrow.

In one aspect, the stimulus of the biocircuit system may be a microenvironment such as, but not limited to, a tumor microenvironment, the cell periphery, the cell membrane, the nuclear membrane, an endosome, a microenvironment characterized by an intracellular or extracellular gradient, and cytoskeletal structures or regions.

In one aspect, the stimulus of the biocircuit system may be the presence of one or more cations and the cation such as, but not limited to, Aluminum, Ammonium, Barium, Calcium, Chromium(II), Chromium(III), Copper(I), Copper(II), Iron(II), Iron(III), Hydrogen, Hydronium, Lead(II), Lithium, Magnesium, Manganese(II), Manganese(III), Mercury(I), Mercury(II), Nitronium, Potassium, Silver, Sodium, Strontium, Tin(II), Tin(IV), and Zinc.

In one aspect, the stimulus of the biocircuit system may be the presence of one or more anions or oxoanions such as, but not limited to, Chloride, Fluoride, Arsenate, Phosphate, Arsenite, Hydrogen phosphate, Dihydrogen phosphate, Sulfate, Nitrate, Hydrogen sulfate, Nitrite, Thiosulfate, Sulfite, Perchlorate, Iodate, Chlorate, Bromate, Chlorite, Hypochlorite, Hypobromite, Carbonate, Chromate, Hydrogen carbonate or Bicarbonate, Dichromate, Acetate, formate, Cyanide, Cyanate, Peroxide, Thiocyanate, Oxalate, Hydroxide, and Permanganate.

In one aspect, the stimulus of the biocircuit system may be the presence of one or more metal ions and the metal ion such as, but not limited to, Magnesium, Manganese, Calcium and Zinc.

The invention also provides polynucleotides encoding an effector module of a biocircuit system described herein. In one aspect, at least one region of the polynucleotide is codon optimized such as, but not limited to, the region encoding the first component of the effector module and/or the region encoding the second component of the effector module.

The polynucleotide encoding an effector module of a biocircuit system may be a DNA molecule. The polynucleotide may have a region such as, but not limited to, a region comprising the sequence of any known microRNAs. The polynucleotide may have a region such as, but not limited to, a region comprising the sequence of any of the microRNAs listed in Table 13 U.S. Patent Application Publication No. 20190192691, the reverse complement of the microRNAs listed in Table 13 U.S. Patent Application Publication No. 20190192691, or the microRNA anti-seed region of any of the microRNAs listed in Table 13 U.S. Patent Application Publication No. 20190192691. Table 13 is incorporated by reference herein in its entirety,

The polynucleotide encoding an effector module of a biocircuit system may be a messenger RNA (mRNA) molecule. The polynucleotide may have a region such as, but not limited to, a region comprising the sequence of any of the microRNAs listed in Table 13 of U.S. Patent Application Publication No. 20190192691, the reverse complement of the microRNAs listed in Table 13 of U.S. Patent Application Publication No. 20190192691, or the microRNA anti-seed region of any of the microRNAs listed in Table 13 of U.S. Patent Application Publication No. 20190192691. The mRNA molecule may comprise one or more chemical modifications such as, but not limited to, a sugar, nucleobase or backbone modifications. The modifications may be naturally or non-naturally occurring modifications.

The invention also provides polynucleotides comprising a first region encoding a stimulus response element (SRE). The SRE may be, but is not limited to, a peptide, a peptide complex, a peptide-protein complex, a protein, a fusion protein, a protein complex, and a protein-protein complex. The SRE may comprise one or more regions derived from a payload, the payload may have a sequence such as, but not limited to, one or more regions derived from a Target of a ligand binding partner pair listed in Table 2 of U.S. Patent Application Publication No. 20190192691, Table 3 of U.S. Patent Application Publication No. 20190192691, or an SRE listed in Table 4 of U.S. Patent Application Publication No. 20190192691.

The polynucleotide may comprise a second region encoding one or more payload constructs. The payload construct may comprise a payload or an antibody or functional fragment thereof. The payload may have a sequence such as, an antibody listed in Table 5 of U.S. Patent Application Publication No. 20190192691 or a functional fragment thereof.

The polynucleotide may also comprise a third region encoding a linker, modifier, signal sequence; binding domain, regulatory motif, dimerization domain, and/or cleavage site.

At least one region of the polynucleotide may be codon optimized. As a non-limiting example, the region encoding the first component of the effector module is codon optimized. As another non-limiting example, the region encoding the second component of the effector module is codon optimized.

The polynucleotide may be a DNA molecule. The polynucleotide may have a region such as, but not limited to, a region comprising the sequence of any known microRNA. The polynucleotide may have a region such as, but not limited to, a region comprising the sequence of any of the microRNAs listed in Table 13 of U.S. Patent Application Publication No. 20190192691, the reverse complement of the microRNAs listed in Table 13 of U.S. Patent Application Publication No. 20190192691, or the microRNA anti-seed region of any of the microRNAs listed in Table 13 of U.S. Patent Application Publication No. 20190192691.

The polynucleotide may be a messenger RNA (mRNA) molecule. The polynucleotide may have a region such as, but not limited to, a region comprising the sequence of any of the known microRNAs. The polynucleotide may have a region such as, but not limited to, a region comprising the sequence of any of the microRNAs listed in Table 13 of U.S. Patent Application Publication No. 20190192691, the reverse complement of the microRNAs listed in Table 13 of U.S. Patent Application Publication No. 20190192691, or the microRNA anti-seed region of any of the microRNAs listed in Table 13 of U.S. Patent Application Publication No. 20190192691. The mRNA molecule may comprise one or more chemical modifications such as, but not limited to, a sugar, nucleobase or backbone modifications. The modifications may be naturally or non-naturally occurring modifications.

The invention also provides an expression vector comprising any of the polynucleotides described herein.

The invention also provides a cell comprising any of the expression vectors described herein.

The invention also provides a transgenic animal comprising any of the cells comprising expression vectors described herein.

The invention also provides a kit comprising any of the polynucleotides or expression vectors described herein.

The invention also provides methods of treating a disease or disorder in a subject, comprising administration of a pharmaceutical composition comprising a biocircuit system or component thereof described herein or the polynucleotides described herein.

In one aspect of the method, the pharmaceutical composition may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

The invention also provides a regulatable human T cell or T cell population engineered to express an effector module. In one aspect, the effector module encodes a chimeric antigen receptor (CAR). In one aspect, the T-cells are primary T-cells. In another aspect, the T cell may be, but is not limited to, cytotoxic T-cells, helper T-cells, memory T-cells, regulatory T-cells, tissue infiltrating lymphocytes and combinations thereof.

In one aspect, the cell population may be obtained from a subject suffering from, being treated for, diagnosed with, at risk of developing, or suspected of having a disorder selected from the group consisting of an immune disorder (including autoimmune disorders), a hypoproliferative condition including cancer, an infectious disease, a non-infectious disease, and graft vs. host disease.

The invention also provides methods of producing a regulatable human T-cell or population thereof by contacting an isolated population of T-cells with a polynucleotide encoding one or more effector modules which can be expressed in the contacted population. The contacting of the isolated population may cause the level of the payload encoded by one or more effector modules to be modulated upon exposure of the expressed effector module with one or more stimuli.

In one aspect, the level of the payload encoded by one or more effector modules may be upregulated upon exposure to the stimulus.

In one aspect, the one or more effector modules encodes at least one cytokine.

In one aspect, the isolated population of T-cells may be contacted with two effector modules. The expressed payload of one of the effector modules may act as a stimulus to the other effector module. Additionally, an isolated population of T-cells may be contacted with a third effector module. The expressed payload of one of the two effector modules can act as a stimulus to the third effector module.

In one aspect, the level of the payload encoded by both of the two effector modules is increased upon exposure to a stimulus.

In another aspect, the level of the payload encoded by one of the two effector modules is increased upon exposure to a stimulus and the level of the payload encoded by the other of the two effector modules is reduced upon exposure to a stimulus.

In another aspect, the exposed stimulus of the first effector module is different from the exposed stimulus of the other effector module.

The invention also provides a method of treating a patient in need, the method comprising administration of the regulatable human T cell or T cell population described herein. The treatment may comprise adoptive immunotherapy.

In one aspect, prior to administration, the regulatable human T cell or T cell population may be expanded.

The invention also provides a multi-tuned effector module responsive to at least one stimulus. The multi-tuned effector module may comprise a first component and a second component, wherein each of the first said second component may independently be but are not limited to, a peptide, peptide complex, peptide-protein complex, protein, fusion protein, protein complex, protein-protein complex.

The first component of the multi-tuned effector module responsive to at least one stimulus may be a stimulus response element (SRE) which comprises one or more regions derived from a payload, one or more regions derived from a Target of a ligand binding partner pair, one or more regions derived from an antibody, or an SRE. The second component of the multi-tuned effector module responsive to at least one stimulus may be a payload construct comprising a payload, a Target of a ligand binding partner pair listed, or an antibody. The payload construct may comprise a polypeptide variant which comprises a cleavage motif, or one or more insertions, deletions, substitutions, additions, or covalent modifications.

The first component of the multi-tuned effector module responsive to at least one stimulus may be a stimulus response element (SRE) which comprises one or more regions derived from a payload, the payload may have a sequence such as, but not limited to, one or more regions derived from a Target of a ligand binding partner pair listed in Table 2 of U.S. Patent Application Publication No. 20190192691, Table 3 of U.S. Patent Application Publication No. 20190192691, one or more regions derived from an antibody listed in Table 5 of U.S. Patent Application Publication No. 20190192691, or an SRE listed in Table 4 of U.S. Patent Application Publication No. 20190192691. The second component of the multi-tuned effector module responsive to at least one stimulus may be a payload construct comprising a payload, the payload may have a sequence such as, but not limited to, a Target of a ligand binding partner pair listed in Table 2 of U.S. Patent Application Publication No. 20190192691, Table 3 of U.S. Patent Application Publication No. 20190192691, or functional fragment thereof; or an antibody listed in Table 5 of U.S. Patent Application Publication No. 20190192691 or a functional fragment thereof of U.S. Patent Application Publication No. 20190192691. The payload construct may comprise a polypeptide variant which comprises a cleavage motif, or one or more insertions, deletions, substitutions, additions, or covalent modifications.

### BRIEF DESCRIPTION OF THE DRAWINGS

FIG. 1 shows an overview diagram of a biocircuit system of the invention. The biocircuit comprises a stimulus and at least one effector module responsive to a stimulus, where the response to the stimulus produces a signal or outcome. The effector module comprises at least one stimulus response element (SRE) and one payload.

FIG. 2 shows representative effector modules carrying one payload. The signal sequence (SS), SRE and payload may be located or positioned in various arrangements without (A to F) or with (G to Z, and AA to DD) a cleavage site. An optional linker may be inserted between each component of the effector module.

FIG. 3 shows representative effector modules carrying two payloads without a cleavage site. The two payloads may be either directly linked to each other or separated.

FIG. 4 shows representative effector modules carrying two payloads with a cleavage site. In one embodiment, an SS is positioned at the N-terminus of the construct, while other components: SRE, two payloads and the cleavage site may be located at different positions (A to L). In another embodiment, the cleavage site is positioned at the N-terminus of the construct (M to X). An optional linker may be inserted between each component of the effector module.

FIG. 5 shows effector modules of the invention carrying two payloads, where an SRE is positioned at the N-terminus of the construct (A to L), while SS, two payloads and the cleavage site can be in any configuration. An optional linker may be inserted between each component of the effector module.

FIG. 6 shows effector modules of the invention carrying two payloads, where either the two payloads (A to F) or one of the two payloads (G to X) is positioned at the N-terminus of the construct (A to L), while SS, SRE and the cleavage site can be in any configuration. An optional linker may be inserted between each component of the effector module.

FIG. 7A and FIG. 7B depict representative configurations of the stimulus and effector module within a biocircuit system. A trans-membrane effector module is activated either by a free stimulus (FIG. 7A) or a membrane bound stimulus (FIG. 7B) which binds to SRE. The response to the stimulus causes the cleavage of the intracellular signal/payload, which activates downstream effector/payload.

FIG. 8 depicts a dual stimulus-dual presenter biocircuit system, where two bound stimuli (A and B) from two different presenters (e.g., different cells) bind to two different effector modules in a single receiver (e.g., another single cell) simultaneously and create a dual-signal to downstream payloads.

FIG. 9 depicts a dual stimulus-single presenter biocircuit system, where two bound stimuli (A and B) from the same presenter (e.g., a single cell) bind to two different effector modules in another single cell simultaneously and create a dual-signal.

FIG. 10 depicts a single-stimulus-bridged receiver biocircuit system. In this configuration, a bound stimulus (A) binds to an effector module in the bridge cell and creates a signal to activate a payload which is a stimulus (B) for another effector module in the final receiver (e.g., another cell).

FIG. 11 depicts a single stimulus-single receiver biocircuit system, wherein the single receiver contains the two effector modules which are sequentially activated by a single stimulus.

FIG. 12 depicts a biocircuit system which requires a dual activation. In this embodiment, one stimulus must bind the transmembrane effector module first to prime the receiver cell being activated by the other stimulus. The receiver only activates when it senses both stimuli (B).

FIG. 13 depicts a standard effector module of a chimeric antigen receptor (CAR) system which comprises an antigen binding domain as an SRE, and signaling domain(s) as payload.

FIG. 14 depicts the structure design of a regulatable CAR system, where the trans-membrane effector modules comprise antigen binding domains sensing an antigen and a first switch domain and the intracellular module comprises a second switch domain and signaling domains. A stimulus (e.g., a dimerization small molecule) can dimerize the first and second switch domains and assemble an activated CAR system.

FIG. 15 shows schematic representation of CAR systems having one (A) or two (B and C) SREs incorporated into the effector module.

FIG. 16A, FIG. 16B, and FIG. 16C depict a split CAR design to control T cell activation by a dual stimulus (e.g., an antigen and small molecule). FIG. 16A shows normal T cell activation which entails a dual activation of TCR and co-stimulatory receptor. The regular CAR design (FIG. 16B) combines the antigen recognition domain with TCR signaling motif and co-stimulatory motif in a single molecule. The split CAR system separates the components of the regular CAR into two separate effector modules which can be reassembled when a heterodimerizing small molecule (stimulus) is present (FIG. 16C).

FIG. 17A and FIG. 17B depict the positive and negative regulation of CAR engineered T cell activation. The absence or presence of a second stimulus can negatively (FIG. 17A) or positively (FIG. 17B) control T cell activation.

FIG. 18A, FIG. 18B, FIG. 18C, and FIG. 18D show schematic representations of gated activation of CAR engineered T cells. If a normal cell that has no stimulus (e.g., an antigen) (FIG. 18A) or an antigen that cannot bind to the trans-membrane effector module (FIG. 18B), or only an antigen that activates the trans-membrane effector module and primes the receiver T cell to express the second effector (FIG. 18C), the receiver T cell remains inactive. When both stimuli (e.g. two antigens) that bind the trans-membrane effector module and the primed effector, are present on the presenter cell (e.g. a cancer cell), the T cell is activated (FIG. 18D).

FIG. 19A and FIG. 19B show representative effector modules having Cas9 or variant Cas9 as the payload and a nuclear localization signal (NLS) without a miR binding site (miR BS) (FIG. 19A) or with a miR binding site (FIG. 19B).

FIG. 20 is a line graph depicting DD-IL2 levels in response to varying concentrations of Shield-1.

FIG. 21A is a bar graph depicting DD-IL12 levels in the various dilutions of media derived from cells expressing DD-IL12. FIG. 21B is a bar graph depicting the Shield-1 dose responsive induction of DD-IL12.

FIG. 22A is a western blot depicting luciferase levels in DD-luciferase expressing cells. FIG. 22B depicts luciferase activity.

FIG. 23A and FIG. 23B are western blots depicting CD3 zeta levels in CD19 CAR expressing cells. FIG. 23C is a western blot depicting 41-BB levels in CD19 CAR expressing cells. FIG. 23D is a bar graph depicting the surface expression of CD19 CAR.

### DETAILED DESCRIPTION OF THE INVENTION

### I. Introduction

Strategies for the conditional regulation of gene expression and protein function have been described in the art and have focused predominantly on the manipulation of gene promoters to alter expression levels. However, such strategies suffer from delays between administration of the stimulus, most often a ligand, and the ultimate response from the system-owing to the delay for signals to reach the nucleus and effect transcriptional changes.

Further, knock down strategies aimed at muting the DNA or destruction of mRNA suffer from a long wait time for the ultimate desired drop in protein levels and function, owing in part to residual protein present and longer protein half-lives for destruction.

The longer lag times between administration of a particular perturbation agent and the response of the natural systems involving transcription, translation and protein degradation limit the application of the methods whose mechanism of action is subject to such natural processing times.

It is more advantageous to target the protein molecule directly. Strategies which directly trigger a cell's natural degradation systems have been developed. One such system relies on temperature sensitivity (for DHFR and the ligand, methotrexate) (Dohmen R J, et al., Science. 1994; 263:1273-1276; and Levy F, et al., Eur. J. Biochem. 1999; 259:244-252).

Others have used reversible systems employing a rapamycin derivative for the regulation of GSK-3β kinase fused to an unstable triple-mutant of the FRB domain (FRB*) (Stankunas et al., Mol Cell. 2003; 12:1615-1624 and Liu et al., Nature. 2007; 446:79-82).

Banaszynski, et al., developed a cell-permeable ligand systems using mutants of FKBP12 protein which were engineered to be unstable in the absence of a high-affinity ligand, Shield-1. (Banaszynski et al., Cell. 2006; 126:995-1004). They termed these unstable domains, destabilizing domains (DDs).

Subsequently, E. coli dihydrofolate reductase (ecDHFR) was explored as a candidate protein from which to design destabilizing domains. One inhibitor of DHFR, trimethoprim (TMP), inhibits ecDHFR much more potently than mammalian DHFR and this differential responsiveness makes this protein-ligand pair ideal for development for use as a biocircuit (Iwamoto, et al., Chem Biol. (2010) September 24; 17(9): 981-988).

Post-translational control has been of great interest in several gene therapy and cell therapy areas including immune-oncology applications and the expanding area of stem cell technology (Reviewed in Rakhit, et al., Chem Biol. 2014 Sep. 18; 21(9): 1238-1252).

Most recently protein switches useful as biosensors as well as new chimeric antigen receptors and other small molecule stabilization frameworks have been disclosed (An W, et al. (2015), PLoS ONE (2015) 10(12): e0145783. doi: 10.1371/journal.pone.0145783; Nicholes, et al., Protein Engineering, Design & Selection, 2016, vol. 29 no. 2, pp. 77-85; Nath, et al., Biochemical and Biophysical Research Communications 470 (2016) 4Ile416); Stevers, et al., PNAS, 2016, vol. 119, no. 9, pp. E112-1161; Juillerat, A. et al., Sci. Rep. (2016), 6, 18950; Roybal, Cell, (2016), vol. 164, pp. 1-10; and Morsut, Cell, (2016), vol. 164, pp. 1-12).

But collectively, these may all be characterized as simple on-off switches, even when combined with one another switch, e.g., the propagation of a single input or effect.

The present invention expands upon the early understandings of destabilizing domain research toward the development of new regulatable biocircuit systems and their methods of use. Such biocircuits include broader spectrum tunable stimulus response elements (SREs) which may be exploited alone or in concert with tunable proteins thus providing increased modularity and flexibility.

### II. Compositions of the Invention

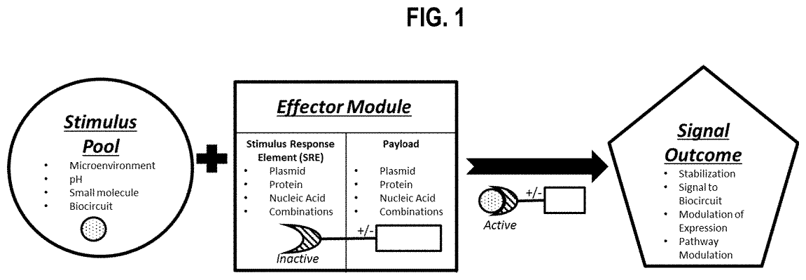

According to the present invention, biocircuit systems are provided which comprise, at their core, at least one effector module system. Such effector module systems comprise at least one effector module having associated, or integral therewith, one or more stimulus response elements (SREs). The overall architecture of a biocircuit system of the invention is illustrated in FIG. 1.

As used herein, a “biocircuit” or “biocircuit system” is defined as a circuit within or useful in biologic systems comprising a stimulus and at least one effector module responsive to a stimulus, where the response to the stimulus produces at least one signal or outcome within, between, as an indicator of, or on a biologic system. Biologic systems are generally understood to be any cell, tissue, organ, organ system or organism, whether animal, plant, fungi, bacterial, or viral. It is also understood that biocircuits may be artificial circuits which employ the stimuli or effector modules taught by the present invention and effect signals or outcomes in acellular environments such as with diagnostic, reporter systems, devices, assays or kits. The artificial circuits may be associated with one or more electronic, magnetic, or radioactive components or parts.

The present invention includes several types of biocircuits including destabilizing domain (DD) biocircuit system, dimer biocircuit systems, chimeric antigen receptor (CAR) biocircuit systems (also known as immune-oncology (I/O biocircuit systems), receptor biocircuit systems, and cell biocircuit systems. Any of these systems may act as a signal to any other of these biocircuit systems.

### Effector Modules, SREs and Payloads

As stated, the biocircuits of the invention include at least one effector module as a component of an effector module system. As used herein, an “effector module” is a single or multi-component construct or complex comprising at least (a) one or more stimulus response elements and (b) one or more payloads.

As used herein a “stimulus response element (SRE)” is a component of an effector module which is joined, attached, linked to or associated with one or more payloads of the effector module and in some instances, is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1% and 100% or a factored increase or decrease such as 2-fold, 3-fold, 4-fold, 5-fold, 10-fold or more.

In some embodiments, the present invention provides methods for modulating protein expression, function or level. In some aspects, the modulation of protein expression, function or level refers to modulation of expression, function or level by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

As used herein a “payload” or “target payload” or “payload of interest (POI)” is defined as any protein or nucleic acid whose function is to be altered.

Payloads may include any coding or non-coding gene or any protein or fragment thereof.

Payloads are often associated with one or more SREs and may be encoded alone or in combination with one or more SRE in a polynucleotide of the invention. Payloads themselves may be altered (at the protein or nucleic acid level) thereby providing for an added layer of tenability of the effector module. For example, payloads may be engineered or designed to contain mutations, single or multiple, which affect the stability of the payload or its susceptibility to degradation, cleavage or trafficking. The combination of an SRE which can have a spectrum of responses to a stimulus with a payload which is altered to exhibit a variety of responses or gradations of output signals, e.g., expression levels, produce biocircuits which are superior to those in the art. For example, mutations or substitutional designs such as those created for IL-12 in WO2016048903 (specifically in Example 1 therein), the contents of which are incorporated herein by reference in their entirety, may be used in any protein payload in conjunction with an SRE of the present invention to create dual tunable biocircuits. The ability to independently tune both the SRE and the payload greatly increases the scope of uses of the effector modules of the present invention.

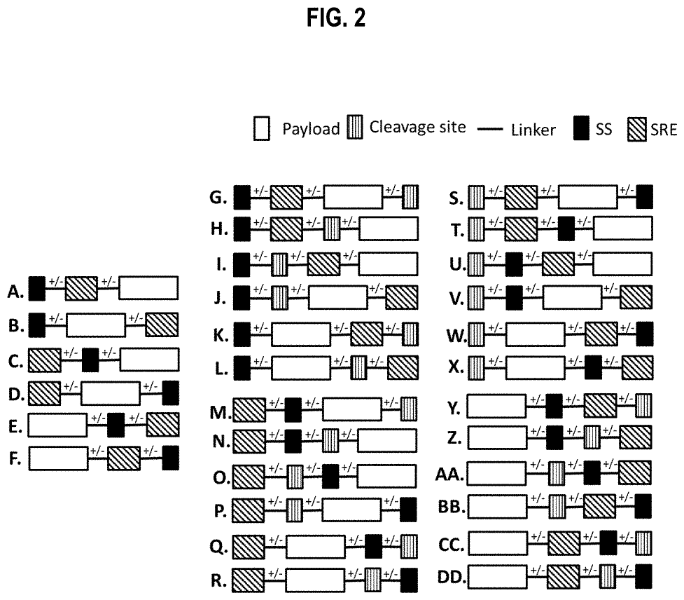

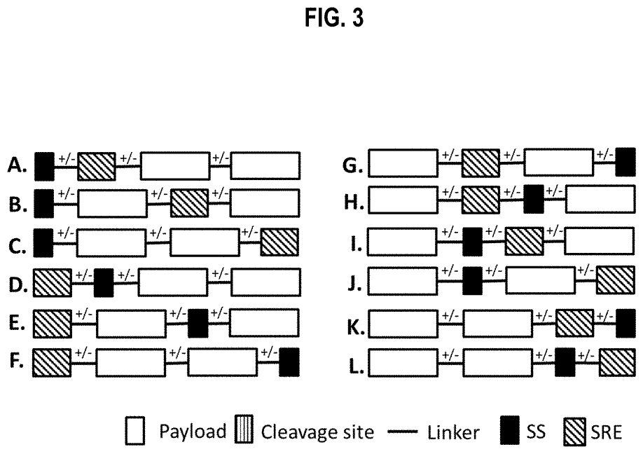

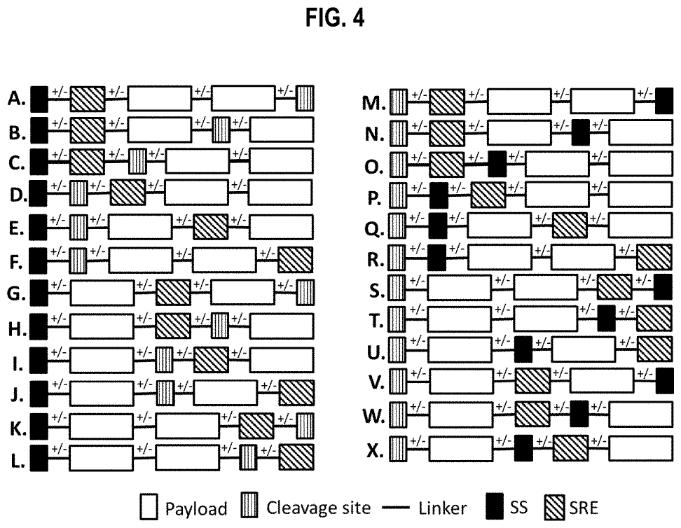

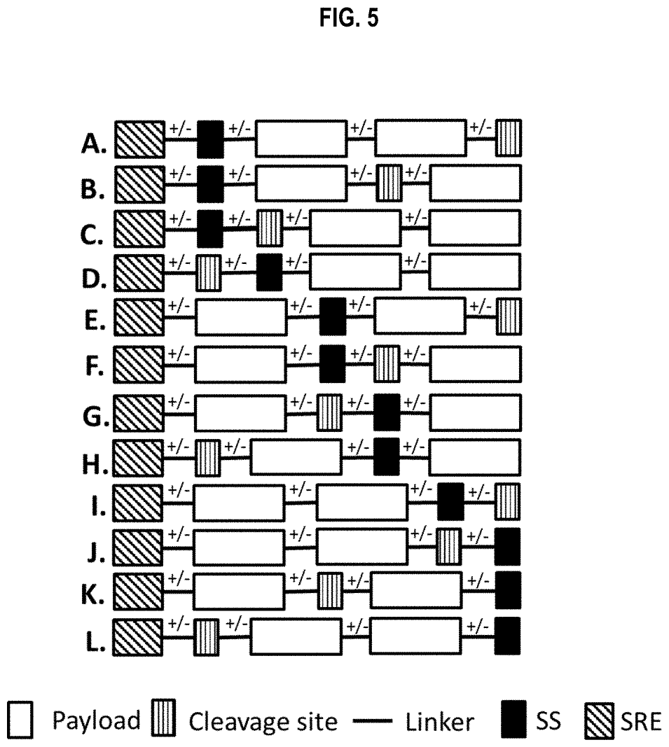

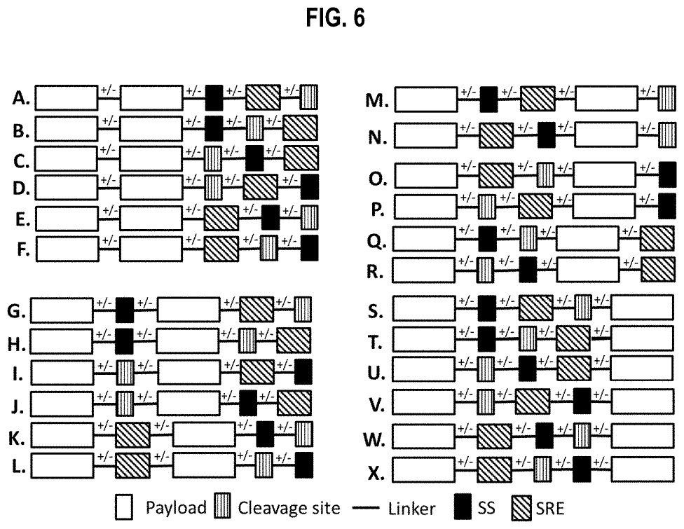

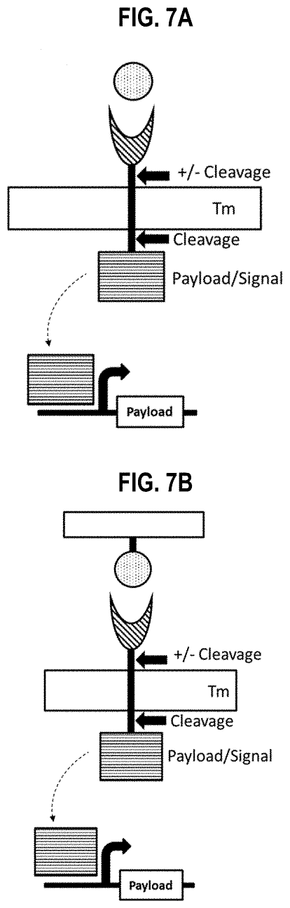

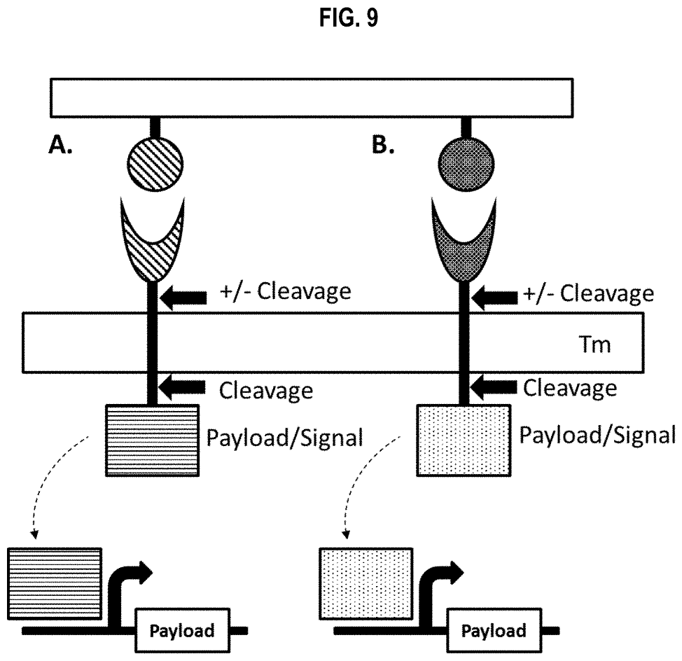

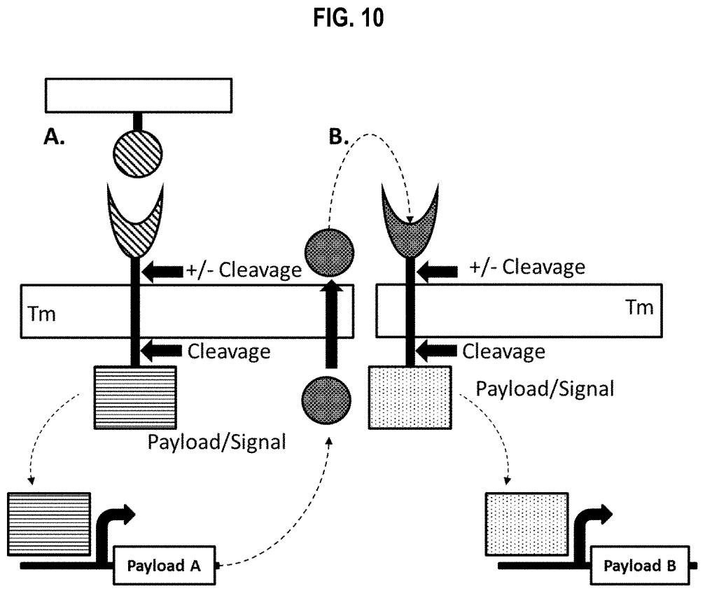

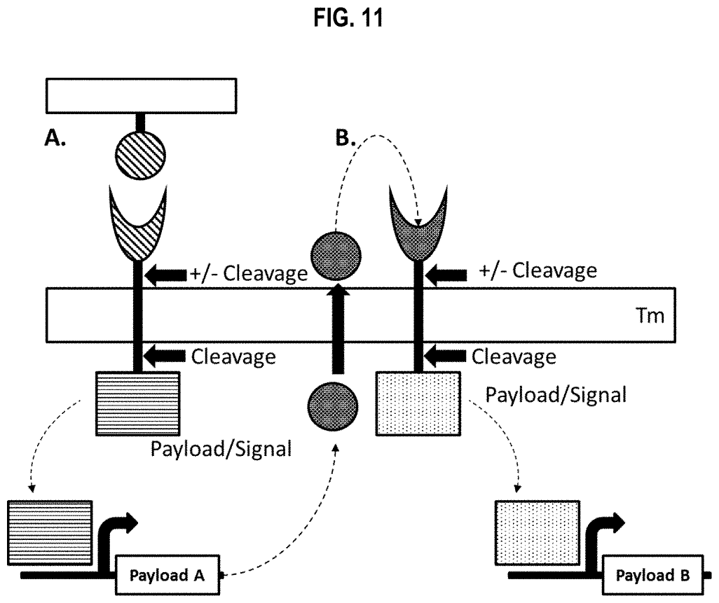

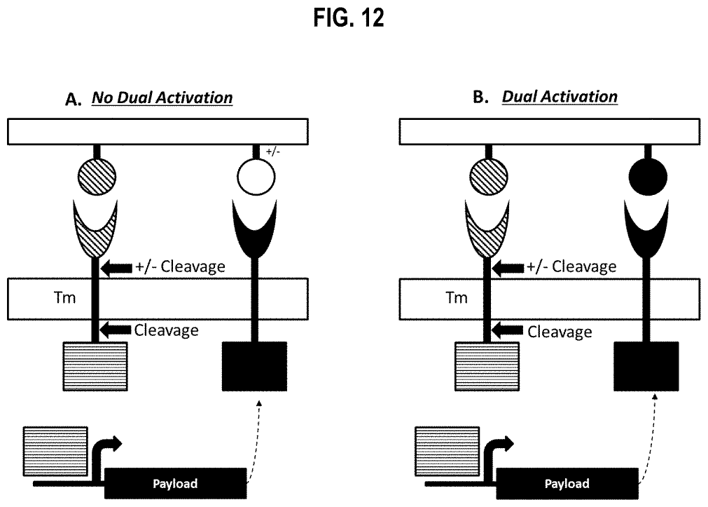

Effector modules may be designed to include one or more payloads, one or more SREs, one or more cleavage sites, one or more signal sequences and one or more additional features including the presence or absence of one or more linkers. Representative effector module embodiments of the invention are illustrated in FIGS. 2-6. Biocircuits and components utilizing such effector molecules are given in FIGS. 7-12.

Effector modules, including their SREs and payloads, may be nucleic acid-based, protein-based or a combination thereof. They may be in the form of DNA, RNA, mRNA, proteins, fusion proteins, or any combination of the foregoing.

Effector modules, including their SREs and payloads may individually, collectively or independently comprise peptides, polypeptides or proteins. At the protein level, such payload may be any natural or artificial peptide or polypeptide or fragment thereof. Natural peptides or polypeptide components of the payload may be derived from any known protein of any species. In some embodiments, they are selected from the payloads represented in Lengthy Table 1 of U.S. Patent Application No. 62/320,864 (Attorney Docket No. 2095.1300USPRO), the contents of which is herein incorporated by reference in its entirety), their coding sequences, or fragments thereof.

Any of the payloads described in U.S. Patent Application Publication No. 20190192691 may be used in the present invention, for example, a cytokine (e.g., IL2, IL12, IL15), a chimeric antigen receptor (e.g., a CAR that recognizes a CD19 antigen) or Cas9, as described in the Examples. The payloads may also comprise any one or more of the ligand binding partners of Tables 2 or 3 of U.S. Patent Application Publication No. 20190192691, any polypeptides taught herein or fragments thereof.

Artificial peptides or polypeptide components of the payload may be derived from any known polypeptide which is not naturally occurring. In some embodiments, they are selected from the antibodies taught in Table 5 of U.S. Patent Application Publication No. 20190192691 or any non-naturally occurring peptides or proteins taught in, for example, Tables 2 and 3 of U.S. Patent Application Publication No. 20190192691, or fragments thereof.

As used herein, the phrase “derived from” as it relates to effector modules, SRE's or payloads means that the effector module, SRE or payload originates at least in part from the stated parent molecule or sequence. For example, in designing an SRE, such SRE may be derived from an epitope or region of a naturally occurring protein but then have been modified in any of the ways taught herein to optimize the SRE function.

Polypeptides of the present invention may comprise amino acid sequences similar to those in Tables 2, 3, or 5 of U.S. Patent Application Publication No. 20190192691, but comprise additional or fewer amino acids than those listed. Such amino acid sequences may comprise about 1 more or fewer amino acids, about 2 more or fewer amino acids, about 3 more or fewer amino acids, about 4 more or fewer amino acids, about 5 more or fewer amino acids, about 6 more or fewer amino acids, about 7 more or fewer amino acids, about 8 more or fewer amino acids, about 9 more or fewer amino acids, about 10 more or fewer amino acids or greater than 10 more or fewer amino acids on N-terminal and/or C-terminal ends.

The stimuli, biocircuit components, effector modules, including their SREs and payloads of the present invention may exist as a whole polypeptide, a plurality of polypeptides or fragments of polypeptides, which independently may be encoded by one or more nucleic acids, a plurality of nucleic acids, fragments of nucleic acids or variants of any of the aforementioned.

As used herein, the term “polypeptide” refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

As used herein, the term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” refers to a variant which contains one or more amino acids which would mimic an activated sequence. For example, glutamate may serve as a mimic for phospho-threonine and/or phospho-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the pharmaceutical compositions, biocircuits, biocircuit components, effector modules including their SREs or payloads of the invention may comprise naturally occurring amino acids and as such may be considered to be proteins, peptides, polypeptides, or fragments thereof. Alternatively, the pharmaceutical compositions, biocircuits, biocircuit components, effector modules including their SREs or payloads may comprise both naturally and non-naturally occurring amino acids.

As used herein, the term “amino acid sequence variant” refers to molecules with some differences in their amino acid sequences as compared to a native or starting sequence. The amino acid sequence variants may possess substitutions, deletions, and/or insertions at certain positions within the amino acid sequence. As used herein, the terms “native” or “starting” when referring to sequences are relative terms referring to an original molecule against which a comparison may be made. Native or starting sequences should not be confused with wild type sequences. Native sequences or molecules may represent the wild-type (that sequence found in nature) but do not have to be identical to the wild-type sequence.

Ordinarily, variants will possess at least about 70% homology to a native sequence, and preferably, they will be at least about 80%, more preferably at least about 90% homologous to a native sequence.

As used herein, the term “homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

As used herein, the term “homolog” as it applies to amino acid sequences is meant the corresponding sequence of other species having substantial identity to a second sequence of a second species.

As used herein, the term “analog” is meant to include polypeptide variants which differ by one or more amino acid alterations, e.g., substitutions, additions or deletions of amino acid residues that still maintain the properties of the parent polypeptide.

As used herein, the term “derivative” is used synonymously with the term “variant” and refers to a molecule that has been modified or changed in any way relative to a reference molecule or starting molecule.

The present invention contemplates several types of pharmaceutical compositions, biocircuits, biocircuit components, effector modules including their SREs or payloads which are amino acid based including variants and derivatives. These include substitutional, insertional, deletional and covalent variants and derivatives. As such, included within the scope of this invention are pharmaceutical compositions, biocircuits, biocircuit components, effector modules including their SREs or payloads comprising substitutions, insertions, additions, deletions and/or covalent modifications. For example, sequence tags or amino acids, such as one or more lysines, can be added to peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein, the term “insertional variants” when referring to proteins are those with one or more amino acids inserted immediately adjacent to an amino acid at a particular position in a native or starting sequence. As used herein, the term “immediately adjacent” refers to an adjacent amino acid that is connected to either the alpha-carboxy or alpha-amino functional group of a starting or reference amino acid.

As used herein, the term “deletional variants” when referring to proteins, are those with one or more amino acids in the native or starting amino acid sequence removed. Ordinarily, deletional variants will have one or more amino acids deleted in a particular region of the molecule.

As used herein, the term “derivatives,” as referred to herein includes variants of a native or starting protein comprising one or more modifications with organic proteinaceous or non-proteinaceous derivatizing agents, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Features of the proteins of the present invention include surface manifestations, local conformational shape, folds, loops, half-loops, domains, half-domains, sites, termini or any combination thereof. As used herein, the term “features” when referring to proteins are defined as distinct amino acid sequence-based components of a molecule.

As used herein, the term “surface manifestation” when referring to proteins refers to a polypeptide based component of a protein appearing on an outermost surface.

As used herein, the term “local conformational shape” when referring to proteins refers to a polypeptide based structural manifestation of a protein which is located within a definable space of the protein.

As used herein, the term “fold,” when referring to proteins, refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein, the term “turn” as it relates to protein conformation, refers to a bend which alters the direction of the backbone of a peptide or polypeptide and may involve one, two, three or more amino acid residues.

As used herein, the term “loop,” when referring to proteins, refers to a structural feature of a peptide or polypeptide which reverses the direction of the backbone of a peptide or polypeptide and comprises four or more amino acid residues. Oliva et al. have identified at least 5 classes of protein loops (Oliva, B. et al., An automated classification of the structure of protein loops. J Mol Biol. 1997. 266(4):814-30.)

As used herein, the term “half-loop,” when referring to proteins, refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein, the term “domain,” when referring to proteins, refers to a motif of a polypeptide having one or more identifiable structural or functional characteristics or properties (e.g., binding capacity, serving as a site for protein-protein interactions.)

As used herein, the term “half-domain,” when referring to proteins, refers to a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, the terms “site,” as it pertains to amino acid based embodiments is used synonymously with “amino acid residue” and “amino acid side chain”. A site represents a position within a peptide or polypeptide that may be modified, manipulated, altered, derivatized or varied within the polypeptide based molecules of the present invention.

As used herein, the terms “termini” or “terminus,” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)).

Polypeptides or proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a biocircuit system component, stimulus, effector module including the SREs or payloads of the invention, any of several manipulations and/or modifications of these features may be performed by moving, swapping, inverting, deleting, randomizing or duplicating. Furthermore, it is understood that manipulation of features may result in the same outcome as a modification to the compositions of the invention. For example, a manipulation which involved deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

In some embodiments, compositions of the present invention may comprise one or more atoms that are isotopes. As used herein, the term “isotope” refers to a chemical element that has one or more additional neutrons. In some embodiments, compounds of the present invention may be deuterated. As used herein, the term “deuterate” refers to the process of replacing one or more hydrogen atoms in a substance with deuterium isotopes. Deuterium isotopes are isotopes of hydrogen. The nucleus of hydrogen contains one proton while deuterium nuclei contain both a proton and a neutron. The pharmaceutical compositions, biocircuits, biocircuit components, effector modules including their SREs or payloads of the present invention may be deuterated in order to change one or more physical property, such as stability, or to allow pharmaceutical compositions, biocircuits, biocircuit components, effector modules including their SREs or payloads to be used in diagnostic and/or experimental applications.

Effector modules may be designed to operate in groups of one, two, three, four or more modules. When more than one effector module is utilized in a biocircuit, it is known as an effector module system of that biocircuit.

At the protein level, any of the biocircuit components may comprise one or more post-translational modifications (PTM). Such PTMs may occur intracellularly after administration of a protein-based biocircuit component or upon or after translation of a biocircuit component administered as a nucleic acid encoding said biocircuit component.

Post translational modifications (PTMs) of the present invention include, but are not limited to acetylation, phosphorylation, ubiquitination, carboxylation, deamidation, deamination, deacetylation, dihydroxylation, dephosphorylation, formylation, gamma-carboxyglutamation, glutathionylation, glycation, hydroxylation, methylation, nitration, sumoylation, N- or O-transglutamination, glycosylation and farnesylation.

Effector modules, including their SREs and payloads, may independently have 1, 2, 3, 4, 5, 6, 7, 8, 9, 10 or more PTMs which are the same or different.

Effector modules may be designed to include one or more structural or functional domain, repeat, or motif of a protein family. Such domains, repeats and motifs are categorized by protein family; and representative families are given in the EMBL-EBI database, located at http://www.ebi.ac.uk/.

### Polynucleotides of the Invention

Biocircuit components including effector modules, their SREs and payloads, may be nucleic acid-based. The term “nucleic acid,” in its broadest sense, includes any compound and/or substance that comprise a polymer of nucleotides, e.g., linked nucleosides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In some embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Polynucleotides of the invention may be mRNA or any nucleic acid molecule and may or may not be chemically modified.

Traditionally, the basic components of an mRNA molecule include at least a coding region, a 5′UTR, a 3′UTR, a 5′ cap and a poly-A tail. Building on this wild type modular structure, the present invention expands the scope of functionality of traditional mRNA molecules by providing payload constructs which maintain a modular organization, but which comprise one or more structural and/or chemical modifications or alterations which impart useful properties to the polynucleotide, for example tenability of function. As used herein, a “structural” feature or modification is one in which two or more linked nucleosides are inserted, deleted, duplicated, inverted or randomized in a polynucleotide without significant chemical modification to the nucleosides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, polynucleotides of the present invention may harbor 5′UTR sequences which play a role in translation initiation. 5′UTR sequences may include features such as Kozak sequences which are commonly known to be involved in the process by which the ribosome initiates translation of genes, Kozak sequences have the consensus XCCR(A/G) CCAUG, where R is a purine (adenine or guanine) three bases upstream of the start codon (AUG) and X is any nucleotide. In one embodiment, the Kozak sequence is ACCGCC. By engineering the features that are typically found in abundantly expressed genes of target cells or tissues, the stability and protein production of the polynucleotides of the invention can be enhanced.

Further provided are polynucleotides, which may contain an internal ribosome entry site (IRES) which play an important role in initiating protein synthesis in the absence of 5′ cap structure in the polynucleotide. An IRES may act as the sole ribosome binding site, or may serve as one of the multiple binding sites. Polynucleotides of the invention containing more than one functional ribosome binding site may encode several peptides or polypeptides that are translated independently by the ribosomes giving rise to bicistronic and/or multicistronic nucleic acid molecules.

In some embodiments, regions of the polynucleotides of the invention which may encode a biocircuit component, effector module, SRE or peptide or protein may include from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

Other regions of the polynucleotides which encode certain features such as for example cleavage sites, trafficking signals such as localization signals or smaller features may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

According to the present invention, multiple distinct biocircuit components, effector modules, their SREs or payloads may be linked together through the 3′-end using nucleotides which are modified at the 3′-terminus. Chemical conjugation may be used to control the stoichiometry of delivery into cells. Polynucleotides encoding biocircuit components, effector modules, their SREs or payloads can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. They may also be conjugated to, administered with, or further encode one or more of RNAi agents, siRNAs, shRNAs, miRNAs, miRNA binding sites, antisense RNAs, ribozymes, catalytic DNA, tRNA, RNAs that induce triple helix formation, aptamers or vectors, and the like.

Once any of the features have been identified or defined as a desired component of a biocircuit, effector modules, their SREs or payloads to be encoded by the polynucleotides the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full-length molecule would.

Modifications and manipulations can be accomplished by methods known in the art such as, but not limited to, site directed mutagenesis. The resulting modified molecules may then be tested for activity using in vitro or in vivo assays such as those described herein or any other suitable screening assay known in the art.

In one embodiment, polynucleotides of the present invention may encode variant polypeptides which have a certain identity with a reference polypeptide sequence. As used herein, a “reference polypeptide sequence” refers to a starting polypeptide sequence. Reference sequences may be wild type sequences or any sequence to which reference is made in the design of another sequence. A “reference polypeptide sequence” may, e.g., be any one of the protein sequences listed in Tables 2, 4 or 5, of U.S. Patent Application Publication No. 20190192691 or any starting polypeptide sequence or fragment thereof.

The term “identity” as known in the art, refers to a relationship between two or more sequences, as determined by comparing the sequences. In the art, identity also means the degree of sequence relatedness between sequences, as determined by the number of matches between strings of two or more residues (amino acid or nucleic acid). Identity measures the percent of identical matches between two or more sequences with gap alignments (if any) addressed by a particular mathematical model or computer program (i.e., “algorithms”). Identity of related sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the variant sequence may have the same or a similar activity as the reference sequence. Alternatively, the variant may have an altered activity (e.g., increased or decreased) relative to a reference sequence. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.)

### RNA Binding Domains

In one embodiment, the polynucleotides of the invention comprise at least one RNA-binding motif such as, but not limited to a RNA-binding domain (RBD).

RNA binding proteins (RBPs) can regulate numerous aspects of co- and post-transcription gene expression such as, but not limited to, RNA splicing, localization, translation, turnover, polyadenylation, capping, modification, export and localization. RNA-binding domains (RBDs), such as, but not limited to, RNA recognition motif (RR) and hnRNP K-homology (KH) domains, typically regulate the sequence association between RBPs and their RNA targets (Ray et al. Nature 2013. 499:172-177; herein incorporated by reference in its entirety). In one embodiment, RBDs can bind short RNA sequences. In another embodiment, the RBDs can recognize structure RNAs.

### Exosome Quantification

In one embodiment, the polynucleotides of the present invention may be quantified in exosomes derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

It is advantageous to correlate the level of polynucleotides with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

### Chemical Modifications to Polynucleotides

According to the present invention, the terms “modification” or, as appropriate, “modified” polynucleotides refer to modification with respect to A, G, U (T in DNA) or C nucleotides.

Modifications of the polynucleotides of the invention may be on the nucleoside base and/or sugar portion of the nucleosides which comprise the polynucleotide. In some embodiments, multiple modifications are included in the modified nucleic acid or in one or more individual nucleoside or nucleotide. For example, modifications to a nucleoside may include one or more modifications to the nucleobase and the sugar. Modifications to the polynucleotides of the present invention may include any of those taught in, for example, International Publication WO2013052523, the contents of which are incorporated herein by reference in its entirety.

As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group.

The modified nucleotides, which may be incorporated into a polynucleotide can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). Other modifications which may be used are taught in, for example, International Application WO2013052523, the contents of which are incorporated herein by reference in their entirety.

### Nucleotide and Nucleosides Modifications

Chemical modifications and/or substitution of the nucleotides or nucleobases of the polynucleotides of the invention which are useful in the present invention include, but are not limited to: (±)1-(2-Hydroxypropyl)pseudouridine TP, (2R)-1-(2-Hydroxypropyl)pseudouridine TP, (2S)-1-(2-Hydroxypropyl)pseudouridine TP, (E)-5-(2-Bromo-vinyl)ara-uridine TP, (E)-5-(2-Bromo-vinyl)cytidine TP, (E)-5-(2-Bromo-vinyl)uridine TP, (Z)-5-(2-Bromo-vinyl)ara-uridine TP, (Z)-5-(2-Bromo-vinyl)uridine TP, 1 (aminoalkylamino-carbonylethylenyl)-2(thio)-pseudouracil, 1 (aminoalkylaminocarbonylethylenyl)-2,4-(dithio)pseudouracil, 1 (aminoalkylaminocarbonylethylenyl)-4 (thio)pseudouracil, 1 (aminoalkylaminocarbonylethylenyl)-pseudouracil, 1 (aminocarbonylethylenyl)-2(thio)-pseudouracil, 1 (aminocarbonylethylenyl)-2,4-(dithio)pseudouracil, 1 (aminocarbonylethylenyl)-4 (thio)pseudouracil, 1 (aminocarbonylethylenyl)-pseudouracil, 1 substituted 2(thio)-pseudouracil, 1 substituted 2,4-(dithio)pseudouracil, 1 substituted 4 (thio)pseudouracil, 1 substituted pseudouracil, 1-(2,2,2-Trifluoroethyl)-pseudo-UTP, 1-(2,2,3,3,3-Pentafluoropropyl)pseudouridine TP, 1-(2,2-Diethoxyethyl)pseudouridine TP, 1-(2,4,6-Trimethylbenzyl)pseudouridine TP, 1-(2,4,6-Trimethyl-benzyl)pseudo-UTP, 1-(2,4,6-Trimethyl-phenyl)pseudo-UTP, 1-(2-Amino-2-carboxyethyl)pseudo-UTP, 1-(2-Amino-ethyl)pseudo-UTP, 1-(2-Hydroxyethyl)pseudouridine TP, 1-(2-Methoxyethyl)pseudouridine TP, 1-(3,4-Bis-trifluoromethoxybenzyl)pseudouridine TP, 1-(3,4-Dimethoxybenzyl)pseudouridine TP, 1-(3-Amino-3-carboxypropyl)pseudo-UTP, 1-(3-Amino-propyl)pseudo-UTP, 1-(3-Cyclopropyl-prop-2-ynyl)pseudouridine TP, 1-(4-Amino-4-carboxybutyl)pseudo-UTP, 1-(4-Amino-benzyl)pseudo-UTP, 1-(4-Amino-butyl)pseudo-UTP, 1-(4-Amino-phenyl)pseudo-UTP, 1-(4-Azidobenzyl)pseudouridine TP, 1-(4-Bromobenzyl)pseudouridine TP, 1-(4-Chlorobenzyl)pseudouridine TP, 1-(4-Fluorobenzyl)pseudouridine TP, 1-(4-Iodobenzyl)pseudouridine TP, 1-(4-Methanesulfonylbenzyl)pseudouridine TP, 1-(4-Methoxybenzyl)pseudouridine TP, 1-(4-Methoxy-benzyl)pseudo-UTP, 1-(4-Methoxy-phenyl)pseudo-UTP, 1-(4-Methylbenzyl)pseudouridine TP, 1-(4-Methyl-benzyl)pseudo-UTP, 1-(4-Nitrobenzyl)pseudouridine TP, 1-(4-Nitro-benzyl)pseudo-UTP, 1(4-Nitro-phenyl)pseudo-UTP, 1-(4-Thiomethoxybenzyl)pseudouridine TP, 1-(4-Trifluoromethoxybenzyl)pseudouridine TP, 1-(4-Trifluoromethylbenzyl)pseudouridine TP, 1-(5-Amino-pentyl)pseudo-UTP, 1-(6-Amino-hexyl)pseudo-UTP, 1-(aminoalkylamino-carbonylethylenyl)-2-(thio)-pseudouracil, 1-(aza)-2-(thio)-3-(aza)-phenoxazin-1-yl, 1,2′-O-dimethyladenosine, 1,2′-O-dimethylguanosine, 1,2′-O-dimethylinosine, 1,3-(diaza)-2-(oxo)-phenthiazin-1-yl, 1,3-(diaza)-2-(oxo)-phenoxazin-1-yl, 1,3,5-(triaza)-2,6-(dioxa)-naphthalene, 1,6-Dimethyl-pseudo-UTP, 1-[3-(2-{2-[2-(2-Aminoethoxy)-ethoxy]-ethoxy}-ethoxy)-propionyl]pseudouridine TP, 1-{3-[2-(2-Aminoethoxy)-ethoxy]-propionyl} pseudouridine TP, 1-Acetylpseudouridine TP, 1-Alkyl-6-(1-propynyl)-pseudo-UTP, 1-Alkyl-6-(2-propynyl)-pseudo-UTP, 1-Alkyl-6-allyl-pseudo-UTP, 1-Alkyl-6-ethynyl-pseudo-UTP, 1-Alkyl-6-homoallyl-pseudo-UTP, 1-Alkyl-6-vinyl-pseudo-UTP, 1-Allylpseudouridine TP, 1-Aminomethyl-pseudo-UTP, 1-Benzoylpseudouridine TP, 1-Benzyloxymethylpseudouridine TP, 1-Benzyl-pseudo-UTP, 1-Biotinyl-PEG2-pseudouridine TP, 1-Biotinylpseudouridine TP, 1-Butyl-pseudo-UTP, 1-carboxymethyl-pseudouridine, 1-Cyanomethylpseudouridine TP, 1-Cyclobutylmethyl-pseudo-UTP, 1-Cyclobutyl-pseudo-UTP, 1-Cycloheptylmethyl-pseudo-UTP, 1-Cycloheptyl-pseudo-UTP, 1-Cyclohexylmethyl-pseudo-UTP, 1-Cyclohexyl-pseudo-UTP, 1-Cyclooctylmethyl-pseudo-UTP, 1-Cyclooctyl-pseudo-UTP, 1-Cyclopentylmethyl-pseudo-UTP, 1-Cyclopentyl-pseudo-UTP, 1-Cyclopropylmethyl-pseudo-UTP, 1-Cyclopropyl-pseudo-UTP, 1-Deazaadenosine TP, 1-Ethyl-pseudo-UTP, 1-Hexyl-pseudo-UTP, 1-Homoallylpseudouridine TP, 1-Hydroxymethylpseudouridine TP, 1-iso-propyl-pseudo-UTP, 1-Me-2-thio-pseudo-UTP, 1-Me-4-thio-pseudo-UTP, 1-Me-alpha-thio-pseudo-UTP, 1-Me-GTP, 1-Methanesulfonylmethylpseudouridine TP, 1-Methoxymethylpseudouridine TP, 1-methyl-1-deaza-pseudoisocytidine, 1-methyl-1-deaza-pseudouridine, 1-Methyl-3-(3-amino-3-carboxypropyl) pseudouridine TP, 1-Methyl-3-(3-amino-3-carboxypropyl)pseudo-UTP, 1-methyl-3-(3-amino-5-carboxypropyl)pseudouridine, 1-Methyl-6-(2,2,2-Trifluoroethyl)pseudo-UTP, 1-Methyl-6-(4-morpholino)-pseudo-UTP, 1-Methyl-6-(4-thiomorpholino)-pseudo-UTP, 1-Methyl-6-(substituted phenyl)pseudo-UTP, 1-Methyl-6-amino-pseudo-UTP, 1-Methyl-6-azido-pseudo-UTP, 1-Methyl-6-bromo-pseudo-UTP, 1-Methyl-6-butyl-pseudo-UTP, 1-Methyl-6-chloro-pseudo-UTP, 1-Methyl-6-cyano-pseudo-UTP, 1-Methyl-6-dimethylamino-pseudo-UTP, 1-Methyl-6-ethoxy-pseudo-UTP, 1-Methyl-6-ethylcarboxylate-pseudo-UTP, 1-Methyl-6-ethyl-pseudo-UTP, 1-Methyl-6-fluoro-pseudo-UTP, 1-Methyl-6-formyl-pseudo-UTP, 1-Methyl-6-hydroxyamino-pseudo-UTP, 1-Methyl-6-hydroxy-pseudo-UTP, 1-Methyl-6-iodo-pseudo-UTP, 1-Methyl-6-iso-propyl-pseudo-UTP, 1-Methyl-6-methoxy-pseudo-UTP, 1-Methyl-6-methylamino-pseudo-UTP, 1-Methyl-6-phenyl-pseudo-UTP, 1-Methyl-6-propyl-pseudo-UTP, 1-Methyl-6-tert-butyl-pseudo-UTP, 1-methyl-6-thio-guanosine, 1-Methyl-6-trifluoromethoxy-pseudo-UTP, 1-Methyl-6-trifluoromethyl-pseudo-UTP, 1-methyladenosine, 1-methylguanosine, 1-methylinosine, 1-methylpseduouridine, 1-methyl-pseudoisocytidine, 1-methyl-pseudouridine, 1-Methyl-pseudo-UTP, 1-Morpholinomethylpseudouridine TP, 1-Pentyl-pseudo-UTP, 1-Phenyl-pseudo-UTP, 1-Pivaloylpseudouridine TP, 1-Propargylpseudouridine TP, 1-Propyl-pseudo-UTP, 1-propynyl-pseudouridine, 1-propynyl-uridine, 1-p-tolyl-pseudo-UTP, 1-taurinomethyl-1-methyl-uridine, 1-taurinomethyl-4-thio-uridine, 1-taurinomethyl-pseudouridine, 1-tert-Butyl-pseudo-UTP, 1-Thiomethoxymethylpseudouridine TP, 1-Thiomorpholinomethylpseudouridine TP, 1-Trifluoroacetylpseudouridine TP, 1-Trifluoromethyl-pseudo-UTP, 1-Vinylpseudouridine TP, 2 (amino)adenine, 2 (amino)purine, 2 (aminopropyl)adenine, 2 (methylthio) N6 (isopentenyl)adenine, 2 (propyl)guanine, 2 (thio)pseudouracil, 2′ deoxy uridine, 2′ fluorouridine, 2-(alkyl)adenine, 2-(alkyl)guanine, 2-(aminoalkyl)adenine, 2-(aminopropyl)adenine, 2-(halo)adenine, 2-(propyl)adenine, 2-(thio)cytosine, 2-(thio)uracil, 2-, 6-, 7- or 8-position of the purine base (A/G/inosine), 2,2′-anhydro-cytidine TP hydrochloride, 2,2′-anhydro-uridine TP, 2,2-dimethyl-guanosine, 2,4-(dithio)psuedouracil, 2,4,5-(trimethyl)phenyl, 2,4-diaminopurine, 2,6-(diamino)purine, 2,6-diaminopurine, 2′ methyl, 2′amino, 2′azido, 2′fluoro-cytidine, 2′ methyl, 2′amino, 2′azido, 2′fluoro-adenine, 2′ methyl, 2′amino, 2′azido, 2′fluoro-guanosine, 2′-Amino-2′-deoxy-ATP, 2′-Amino-2′-deoxy-CTP, 2′-Amino-2′-deoxy-GTP, 2′-Amino-2′-deoxy-UTP, 2′-Azido-2′-deoxy-ATP, 2′-Azido-2′-deoxy-CTP, 2′-Azido-2′-deoxy-GTP, 2′-Azido-2′-deoxy-UTP, 2′-Azido-deoxyuridine TP, 2′-bromo-deoxyuridine TP, 2′-F-5-Methyl-2′-deoxy-UTP, 2′Fluor-N4-Bz-cytidine TP, 2′Fluoro-N2-isobutyl-guanosine TP, 2′Fluoro-N4-Acetyl-cytidine TP, 2′Fluoro-N6-Bz-deoxyadenosine TP, 2′Fluro-N2-isobutyl-guanosine TP, 2′methyl, 2′amino, 2′azido, 2′fluoro-uridine, 2′-OMe-2-Amino-ATP, 2′-OMe-5-Me-UTP, 2′-OMe-6-Me-UTP, 2′-OMe-pseudo-UTP, 2′-O-methyladenosine, 2′O-methyl-N2-isobutyl-guanosine TP, 2′-O-Methyl-N4-Acetyl-cytidine TP, 2′O-methyl-N4-Bz-cytidine TP, 2′O-methyl-N6-Bz-deoxyadenosine TP, 2′-O-methylpseudouridine, 2′-O-methyluridine, 2′ deoxy uridine, 2′ fluorouridine, 2′-O-methyladenosine, 2′-O-methylcytidine, 2′-O-methylguanosine, 2′-O-methyluridine, 2′-a-Ethynyladenosine TP, 2′-a-Ethynylcytidine TP, 2′-a-Ethynylguanosine TP, 2′-a-Ethynyluridine TP, 2′-amino-2′-deoxy-, 2′-amino-2′-deoxyadenosine, 2′-amino-2′-deoxycytidine, 2′-amino-2′-deoxyguanosine, 2′-amino-2′-deoxyribose, 2′-amino-2′-deoxyuridine, 2-amino-6-Chloro-purine, 2-Amino-A/U/G/C, 2-aminoadenine, 2-Aminoadenosine TP, 2-Amino-ATP, 2′-aminopropargyl, 2-aminopurine, 2-Amino-riboside-TP, 2′-araadenosine, 2′-aracytidine, 2′-arauridine, 2′-a-Trifluoromethyladenosine TP, 2′-a-Trifluoromethylcytidine TP, 2′-a-Trifluoromethylguanosine TP, 2′-a-Trifluoromethyluridine TP, 2-aza-inosinyl, 2′-azido-2′-deoxyuridine, 2′-Azido-2′-deoxyadenosine, 2′-azido-2′-deoxycytidine, 2′-azido-2′-deoxyguanosine, 2′-azido-2′-deoxyribose, 2′-azido-2′-deoxyuridine, 2′-Azido-2a-deoxyadenosine, 2-Azidoadenosine TP, 2′-b-Ethynyladenosine TP, 2′-b-Ethynylcytidine TP, 2′-b-Ethynylguanosine TP, 2′-b-Ethynyluridine TP, 2-Bromoadenosine TP, 2′-b-Trifluoromethyladenosine TP, 2′-b-Trifluoromethylcytidine TP, 2′-b-Trifluoromethylguanosine TP, 2′-b-Trifluoromethyluridine TP, 2′-C-alkyl oligoribonucleotide, 2-Chloroadenosine TP, 2′-deoxy-uridines, 2′-Deoxy-2′,2′-difluoroadenosine TP, 2′-Deoxy-2′,2′-difluorocytidine TP, 2′-Deoxy-2′,2′-difluoroguanosine TP, 2′-Deoxy-2′,2′-difluorouridine TP, 2′-Deoxy-2′-a-aminoadenosine TP, 2′-Deoxy-2′-a-aminocytidine TP, 2′-Deoxy-2′-a-aminoguanosine TP, 2′-Deoxy-2′-a-aminouridine TP, 2′-Deoxy-2′-a-azidoadenosine TP, 2′-Deoxy-2′-a-azidocytidine TP, 2′-Deoxy-2′-a-azidoguanosine TP, 2′-Deoxy-2′-a-azidouridine TP, 2′-Deoxy-2′-a-mercaptoadenosine TP, 2′-Deoxy-2′-a-mercaptocytidine TP, 2′-Deoxy-2′-a-mercaptoguanosine TP, 2′-Deoxy-2′-a-mercaptouridine TP, 2′-Deoxy-2′-a-thiomethoxyadenosine TP, 2′-Deoxy-2′-a-thiomethoxycytidine TP, 2′-Deoxy-2′-a-thiomethoxyguanosine TP, 2′-Deoxy-2′-a-thiomethoxyuridine TP, 2′-Deoxy-2′-b-aminoadenosine TP, 2′-Deoxy-2′-b-aminocytidine TP, 2′-Deoxy-2′-b-aminoguanosine TP, 2′-Deoxy-2′-b-aminouridine TP, 2′-Deoxy-2′-b-azidoadenosine TP, 2′-Deoxy-2′-b-azidocytidine TP, 2′-Deoxy-2′-b-azidoguanosine TP, 2′-Deoxy-2′-b-azidouridine TP, 2′-Deoxy-2′-b-bromoadenosine TP, 2′-Deoxy-2′-b-bromocytidine TP, 2′-Deoxy-2′-b-bromoguanosine TP, 2′-Deoxy-2′-b-bromouridine TP, 2′-Deoxy-2′-b-chloroadenosine TP, 2′-Deoxy-2′-b-chlorocytidine TP, 2′-Deoxy-2′-b-chloroguanosine TP, 2′-Deoxy-2′-b-chlorouridine TP, 2′-Deoxy-2′-b-fluoroadenosine TP, 2′-Deoxy-2′-b-fluorocytidine TP, 2′-Deoxy-2′-b-fluoroguanosine TP, 2′-Deoxy-2′-b-fluorouridine TP, 2′-Deoxy-2′-b-iodoadenosine TP, 2′-Deoxy-2′-b-iodocytidine TP, 2′-Deoxy-2′-b-iodoguanosine TP, 2′-Deoxy-2′-b-iodouridine TP, 2′-Deoxy-2′-b-mercaptoadenosine TP, 2′-Deoxy-2′-b-mercaptocytidine TP, 2′-Deoxy-2′-b-mercaptoguanosine TP, 2′-Deoxy-2′-b-mercaptouridine TP, 2′-Deoxy-2′-b-thiomethoxyadenosine TP, 2′-Deoxy-2′-b-thiomethoxycytidine TP, 2′-Deoxy-2′-b-thiomethoxyguanosine TP, 2′-Deoxy-2′-b-thiomethoxyuridine TP, 2′-deoxy-2′-C-alkyl oligoribonucleotide, 2′-deoxy-2′-deamine oligoribonucleotide, 2′-deoxy-2′-fluoro-oligoribonucleotide, 2′-deoxy-A/U/G/C, 2′-deoxyuridine, 2′-fluoro-uridines, 2′-fluoro-2′-deoxy, 2′-fluoro-2′-deoxyadenosine, 2′-fluoro-2′-deoxycytidine, 2′-fluoro-2′-deoxyguanosine, 2′fluoro-2′-deoxyribose, 2′-fluoro-2′-deoxyuridine, 2′-fluoro-A/U/G/C, 2-Fluoroadenosine TP, 2′fluoroC/2thioU, 2′-fluoro-modified bases, 2′-fluorothymidine, 2-Iodoadenosine TP, 2′MeA/2thioU, 2′MeC/2thioU, 2′MeG/2thioU, 2-Mercaptoadenosine TP, 2-methoxy-4-thio-pseudouridine, 2-methoxy-4-thio-uridine, 2-methoxy-5-methyl-cytidine, 2-methoxy-adenine, 2-methoxy-cytidine, 2′-methoxyethyl, 2-methoxyuridine, 2-methyladenosine, 2-methyl-guanosine, 2-methylpseudouridine, 2-methylthio-adenine, 2-methylthio-N6-(cis-hydroxyisopentenyl)adenosine, 2-methylthio-N6-hydroxynorvalyl carbamoyladenosine, 2-methylthio-N6-methyladenosine, 2-methylthio-N6-threonyl carbamoyladenosine, 2′-methyluridine, 2′-O-methyl-2′-deoxyguanosine, 2′-O-(3-aminopropyl), 2′-O-alkenyl-A/U/G/C, 2′-O-alkinyl-A/U/G/C, 2′-O-alkyl-oligoribonucleotide, 2′-O-allyl-A/U/G/C, 2′-O-butyl-A/U/G/C, 2′-O-fluoro (2′-OF), 2′-OH-ara-adenosine TP, 2′-OH-ara-cytidine TP, 2′-OH-ara-guanosine TP, 2′-OH-ara-uridine TP, 2′-O-methyl (2′-OMe), 2′-O-methyl adenosine, 2′-O-methyl guanosine, 2′-O-Methyl inosine, 2′-O-methyl uridine, 2′-O-methyl-2-aminoadenosine, 2′-O-methyl-2′-deoxy-, 2′-O-Methyl-2′-deoxyadenosine, 2′-O-methyl-2′-deoxycytidine, 2′-O-Methyl-2′-deoxyguanosine, 2′-O-methyl-2′-deoxyuridine, 2′-O-Methyl-5-(1-propynyl)cytidine TP, 2′-O-Methyl-5-(1-propynyl)uridine TP, 2′-O-methyl-5-methyluridine, 2′-O-methylinosine, 2′-O-methylpseudouridine, 2′-O-methyl-ribose, 2′-O-propyl-A/U/G/C, 2′-O-ribosyladenosine (phosphate), 2′-O-ribosylguanosine (phosphate), 2-oxo-7-aminopyridopyrimidin-3-yl, 2-oxo-pyridopyrimidine-3-yl, 2′-position of the sugar of adenosine, 2′-position of the sugar of cytidine, 2′-position of the sugar of guanosine, 2′-position of the sugar of inosine, 2′-position of the sugar of uridine, 2′-propinyl-A/U/G/C, 2-pyridinone, 2-thio-1-methyl-1-deaza-pseudouridine, 2-thio-1-methyl-pseudouridine, 2-thio-2′-O-methyluridine, 2-thio-5-aza-uridine, 2-thio-5-methyl-cytidine, 2-thiocytidine, 2-thio-dihydropseudouridine, 2-thio-dihydrouridine, 2-thio-pseudouridine, 2thioU/2′aminoC, 2thioU/2′azidoG, 2-thiouridine, 2-thio-zebularine, 2-Trifluoromethyladenosine TP, 3 (3 amino-3 carboxypropyl)uracil, 3 (deaza) 5 (aza)cytosine, 3 (methyl)cytosine, 3 nitropyrrole, 3-(3-amino-3-carboxypropyl)uridine, 3-(3-Amino-3-carboxypropyl)-Uridine TP, 3-(alkyl)cytosine, 3-(deaza) 5 (aza)cytosine, 3-(methyl)-7-(propynyl)isocarbostyrilyl, 3-(methyl)cytidine, 3-(methyl)isocarbostyrilyl, 3,2′-O-dimethyluridine, 3,2′-O-Dimethyluridine TP, 3-Alkyl-pseudo-UTP, 3-Deaza-3-bromoadenosine TP, 3-Deaza-3-chloroadenosine TP, 3-Deaza-3-fluoroadenosine TP, 3-Deaza-3-iodoadenosine TP, 3-Deazaadenosine TP, 3′-Ethynylcytidine TP, 3-methylcytidine, 3-Methyl-pseudo-Uridine TP, 3-methyluridine, 4 (thio)pseudouracil, 4-(fluoro)-6-(methyl)benzimidazole, 4-(methyl)benzimidazole, 4-(methyl)indolyl, 4-(thio)pseudouracil, 4-(thio)uracil, 4-, 5- or 6-position of the pyrimidine base (C/U), 4,2′-O-dimethylcytidine, 4,6-(dimethyl)indolyl, 4-acetyl-cytosine, 4′-Azidoadenosine TP, 4′-Azidocytidine TP, 4′-Azidoguanosine TP, 4′-Azidouridine TP, 4′-Carbocyclic adenosine TP, 4′-Carbocyclic cytidine TP, 4′-Carbocyclic guanosine TP, 4′-Carbocyclic uridine TP, 4-demethylwyosine, 4′-Ethynyladenosine TP, 4′-Ethynylcytidine TP, 4′-Ethynylguanosine TP, 4′-Ethynyluridine TP, 4-methoxy-1-methyl-pseudoisocytidine, 4-methoxy-2-thio-pseudouridine, 4-methoxy-pseudoisocytidine, 4-methoxy-pseudouridine, 4-methylcytidine, 4-thio-1-methyl-1-deaza-pseudoisocytidine, 4-thio-1-methyl-pseudoisocytidine, 4-thio-1-methyl-pseudouridine, 4-thio-A/U/G/C, 4-thio-pseudoisocytidine, 4-thio-pseudouridine, 4-Thio-pseudo-UTP 4thioU/2thioU, 4-thiouracil, 4-thiouridine, 5 (1,3-diazole-1-alkyl)uracil, 5 (2-aminopropyl)uracil, 5 (aminoalkyl)uracil, 5 (dimethylaminoalkyl)uracil, 5 (guanidiniumalkyl)uracil, 5 (halo)cytosine, 5 (methoxycarbonylmethyl)-2-(thio)uracil, 5 (methoxycarbonyl-methyl)uracil, 5 (methyl) 2 (thio)uracil, 5 (methyl) 2,4 (dithio)uracil, 5 (methyl) 4 (thio)uracil, 5 (methyl)cytosine, 5 (methylaminomethyl)-2 (thio)uracil, 5 (methylaminomethyl)-2,4 (dithio)uracil, 5 (methylaminomethyl)-4 (thio)uracil, 5 (propynyl)cytosine, 5 (propynyl)uracil, 5 (trifluoromethyl)cytosine, 5 (trifluoromethyl)uracil, 5 nitroindole, 5 substituted pyrimidines, 5-(1-Propynyl)ara-cytidine TP, 5-(1-Propynyl)ara-uridine TP, 5-(2-aminopropyl)uracil, 5-(2-carbomethoxyvinyl)uridine TP, 5-(2-Chloro-phenyl)-2-thiocytidine TP, 5-(2-Furanyl)uridine TP, 5-(4-Amino-phenyl)-2-thiocytidine TP, 5-(alkyl)-2-(thio)pseudouracil, 5-(alkyl)-2,4 (dithio)pseudouracil, 5-(alkyl)-4 (thio)pseudouracil, 5-(alkyl)cytosine, 5-(alkyl)pseudouracil, 5-(alkyl)uracil, 5-(alkynyl)cytosine, 5-(alkynyl)uracil, 5-(allylamino)uracil, 5-(C1-C6)-alkylcytosine, 5-(C1-C6)-alkyluracil, 5-(C2-C6)-alkenylcytosine, 5-(C2-C6)-alkenyluracil, 5-(C2-C6)-alkynylcytosine, 5-(C2-C6)-alkynyluracil, 5-(carboxyhydroxymethyl)pyrimidine methyl ester-, 5-(carboxyhydroxymethyl)uridine, 5-(carboxyhydroxymethyl)uridine methyl ester, 5-(cyanoalkyl)uracil, 5-(dialkylaminoalkyl)uracil, 5-(dimethylaminoalkyl)uracil, 5-(guanidiniumalkyl)uracil, 5-(halo)cytosine, 5-(halo)uracil, 5-(hydroxymethyl)uracil, 5-(iso-Pentenylaminomethyl)-2-thiouridine TP, 5-(iso-Pentenylaminomethyl)-2′-O-methyluridine TP, 5-(iso-Pentenylaminomethyl)uridine TP, 5-(1,3-diazole-1-alkyl)uracil, 5-(methoxy)uracil, 5-(methoxycarbonylmethyl)-2-(thio)uracil, 5-(methoxycarbonyl-methyl)uracil, 5-(methyl) 2(thio)uracil, 5-(methyl) 2,4 (dithio)uracil, 5-(methyl) 4 (thio)uracil, 5-(methyl)-2-(thio)pseudouracil, 5-(methyl)-2,4 (dithio)pseudouracil, 5-(methyl)-4 (thio)pseudouracil, 5-(methyl)isocarbostyrilyl, 5-(methyl)pseudouracil, 5-(methylaminomethyl)-2 (thio)uracil, 5-(methylaminomethyl)-2,4(dithio)uracil, 5-(methylaminomethyl)-4-(thio)uracil, 5-(propynyl)cytosine, 5-(propynyl)uracil, 5-(trifluoromethyl)cytosine, 5-(trifluoromethyl)uracil, 5,2′-O-dimethylcytidine, 5,2′-O-dimethyluridine, 5,6-dihydro-uridine, 5-aminoallyl-A/U/G/C, 5-Aminoallyl-CTP, 5-Aminoallyl-deoxy-uridine, 5-aminoallyl-uridine, 5-aminomethyl-2-seleno-A/U/G/C, 5-aminomethyl-2-thio-A/U/G/C, 5-aminomethyl-2-thiouridine, 5-aminomethyl-A/U/G/C, 5-aminopropyl-2′-amino-A/U/G/C, 5-aminopropyl-2′-deoxy-A/U/G/C, 5-aminopropyl-2′-fluoro-A/U/G/C, 5-aminopropyl-2′-O-methyl-A/U/G/C, 5-aminopropyl-A/U/G/C, 5-aminouracil, 5-aza-2-thio-zebularine, 5-aza-cytidine, 5-aza-uridine, 5-aza-zebularine, 5-Bromo-2′-deoxyuridine, 5-Bromo-2′-deoxycytidine, 5-bromo-A/U/G/C, 5-bromo-cytidine, 5-bromo-uridine, 5-carbamoylmethyl-2′-O-methyluridine, 5-carbamoylmethyl-A/U/G/C, 5-carbamoylmethyluridine, 5-Carbamoylmethyluridine TP, 5-carboxyhydroxymethyl-A/U/G/C, 5-carboxyhydroxymethyluridine, 5-carboxyhydroxymethyluridine methyl ester, 5-carboxymethyl-A/U/G/C, 5-carboxymethylaminomethyl-2′-O-methyluridine, 5-carboxymethylaminomethyl-2-thio-A/U/G/C, 5-carboxymethylaminomethyl-2-thiouridine, 5-carboxymethylaminomethyl-A/U/G/C, 5-carboxymethylaminomethyluridine, 5-carboxymethyluridine, 5-chloro-ara-cytosine, 5-chlorocytosine, 5-chlorouracil, 5-Crbamoylmethyluridine TP, 5-Cyanocytidine TP, 5-Cyanouridine TP, 5-Dimethylaminouridine TP, 5-Ethynylara-cytidine TP, 5-Ethynylcytidine TP, 5-fluoro-A/U/G/C, 5-fluorocytosine, 5-fluoro-uridine, 5-formyl-2′-O-methylcytidine, 5-formyl-A/U/G/C, 5-formylcytidine, 5′-Homo-adenosine TP, 5′-Homo-cytidine TP, 5′-Homo-guanosine TP, 5′-Homo-uridine TP, 5-hydro-A/U/G/C, 5-hydroxy-A/U/G/C, 5-hydroxycytosine, 5-hydroxydeoxycytidine, 5-hydroxymethyl-A/U/G/C, 5-hydroxymethylcytidine, 5-hydroxymethyldeoxycytidine, 5-hydroxyuridine, 5-iodo-2′-deoxycytidine, 5-iodo-2′-fluoro-deoxyuridine TP, 5-iodo-2′-deoxycytidine, 5-iodo-2′-deoxyuridine, 5-iodo-A/U/G/C, 5-iodocytidine, 5-iodoU/2thioU, 5-iodoU/4thioU, 5-iodo-uridine, 5-isopentenylaminomethyl-2′-amino-A/U/G/C, 5-isopentenylaminomethyl-2′-deoxy-A/U/G/C, 5-isopentenylaminomethyl-2′-fluoro-A/U/G/C, 5-isopentenylaminomethyl-2′-O-methyl-A/U/G/C, 5-isopentenylaminomethyl-2-thio-2′-amino-A/U/G/C, 5-isopentenylaminomethyl-2-thio-2′-deoxy-A/U/G/C, 5-isopentenylaminomethyl-2-thio-2′-fluoro-A/U/G/C, 5-isopentenylaminomethyl-2-thio-2′-O-methyl-A/U/G/C, 5-isopentenylaminomethyl-2-thio-A/U/G/C, 5-isopentenylaminomethyl-A/U/G/C, 5mC/2thioU, 5mC/2thioU/2′aminoC, 5mC/2thioU/2′azidoG, 5mC/4thioU, 5mC/pseudoU, 5-methoxy-A/U/G/C, 5-methoxyaminomethyl-2-thio-uridine, 5-methoxycarbonyl methyl-A/U/G/C, 5-methoxycarbonylmethyl-2′-O-methyluridine, 5-methoxycarbonylmethyl-2-thioA/U/G/C, 5-methoxycarbonylmethyl-2-thiouridine, 5-methoxycarbonylmethyluridine, 5-Methoxycytidine TP, 5-methoxy-ethoxy-methyl-2′-amino-A/U/G/C, 5-methoxy-ethoxy-methyl-2′-deoxy-A/U/G/C, 5-methoxy-ethoxy-methyl-2′-fluoro-A/U/G/C, 5-methoxy-ethoxy-methyl-2′-O-methyl-A/U/G/C, 5-methoxy-ethoxy-methyl-A/U/G/C, 5-methoxyuridine, 5-methyl-2-thio-A/U/G/C, 5-methyl-2-thiouridine, 5-methyl-A/U/G/C, 5-methylaminomethyl-2-selenouridine, 5-methylaminomethyl-2-thiouridine, 5-methylaminomethyl-A/U/G/C, 5-methylaminomethyluridine, 5-methylcytosine, 5-methyldihydro-A/U/G/C, 5-Methyldihydrouridine, 5-methyluridine, 5-methyl-zebularine, 5-nitro-A/U/G/C, 5-nitroindole, 5-oxyacetic acid methyl ester-A/U/G/C, 5-Oxyacetic acid-Uridine TP, 5-oxyacetic acid-A/U/G/C, 5-Oxyacetic acid-methyl ester-Uridine TP, 5-Phenylethynyluridine TP, 5-propynyl cytosine, 5-propynyl uracil, 5-Propynyl-2′-deoxyuridine, 5-Propynyl-2′-deoxycytidine, 5-propynyl-A/U/G/C, 5-taurinomethyl)-2-thiouridine-2′-deoxy-A/U/G/C, 5-taurinomethyl-2′-amino-A/U/G/C, 5-taurinomethyl-2′-fluoro-A/U/G/C, 5-taurinomethyl-2′-O-methyl-A/U/G/C, 5-taurinomethyl-2-thiouridine, 5-taurinomethyl-2-thiouridine-2′-amino-A/U/G/C, 5-taurinomethyl-2-thiouridine-2′-fluoro-A/U/G/C, 5-taurinomethyl-2-thiouridine-2′-O-methyl-A/U/G/C, 5-taurinomethyl-A/U/G/C, 5-taurinomethyluridine, 5-Trideuteromethyl-6-deuterouridine TP, 5-TrifluoromethylCytidine TP, 5-Trifluoromethyl-Uridine TP, 5-uracil, 5-Vinylarauridine TP, 6 (alkyl)adenine, 6 (azo)uracil, 6 (methyl)adenine, 6 (methyl)guanine, 6-(2,2,2-Trifluoroethyl)-pseudo-UTP, 6-(4-Morpholino)-pseudo-UTP, 6-(4-Thiomorpholino)-pseudo-UTP, 6-(alkyl)adenine, 6-(alkyl)guanine, 6-(aza)pyrimidine, 6-(azo)cytosine, 6-(azo)thymine, 6-(azo)uracil, 6-(methyl)-7-(aza)indolyl, 6-(methyl)adenine, 6-(methyl)guanine, 6-(Substituted-Phenyl)-pseudo-UTP, 6-Amino-pseudo-UTP, 6-aza-2′-amino-A/U/G/C, 6-aza-2′-deoxy-A/U/G/C, 6-aza-2′-fluoro-A/U/G/C, 6-aza-2′-O-methyl-A/U/G/C, 6-aza-A/U/G/C, 6-aza-cytidine, 6-azauridine, 6-aza-uridine, 6-Azido-pseudo-UTP, 6-Bromo-pseudo-UTP, 6-Butyl-pseudo-UTP, 6-chloro-7-deaza-guanosine, 6-Chloro-pseudo-UTP, 6-chloro-purine, 6-Cyano-pseudo-UTP, 6-Dimethylamino-pseudo-UTP, 6-Ethoxy-pseudo-UTP, 6-Ethylcarboxylate-pseudo-UTP, 6-Ethyl-pseudo-UTP, 6-Fluoro-pseudo-UTP, 6-Formyl-pseudo-UTP, 6-Hydroxyamino-pseudo-UTP, 6-Hydroxy-pseudo-UTP, 6-Iodo-pseudo-UTP, 6-iso-Propyl-pseudo-UTP, 6-mercapto-guanosine, 6-methoxy-guanosine, 6-Methoxy-pseudo-UTP, 6-methyladenosine, 6-Methylamino-pseudo-UTP, 6-methyl-guanosine, 6-methyl-mercaptopurine, 6-Methyl-pseudo-UTP, 6-Phenyl-pseudo-UTP, 6-Phenyl-pseudo-UTP, 6-phenyl-pyrrolo-pyrimidin-2-on-3-yl, 6-Propyl-pseudo-UTP, 6-tert-Butyl-pseudo-UTP, 6-thio-7-deaza-8-aza-guanosine, 6-thio-7-deaza-guanosine, 6-thio-7-methyl-guanosine, 6-thiodeoxyguanosine, 6-thio-guanosine, 6-Trifluoromethoxy-pseudo-UTP, 6-Trifluoromethyl-pseudo-UTP, 7 (alkyl)guanine, 7 (deaza)adenine, 7 (deaza)guanine, 7 (methyl)guanine, 7-(alkyl)guanine, 7-(aminoalkylhydroxy)-1-(aza)-2-(thio)-3-(aza)-phenthiazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenoxazin-1-yl, 7-(aminoalkylhydroxy)-1,3-(diaza)-2-(oxo)-phenthiazin-1-yl, 7-(aminoalkylhydroxy)-1,3-(diaza)-2-(oxo)-phenthiazin-1-yl, 7-(aminoalkylhydroxy)-1,3-(diaza)-2-(oxo)-phenoxazin-1-yl, 7-(aminoalkylhydroxy)-1,3-(diaza)-2-(oxo)-phenoxazin-1-yl, 7-(aza)indolyl, 7-(deaza)guanine, 7-(guanidiniumalkylhydroxy)-1-(aza)-2-(thio)-3-(aza)-phenoxazinl-yl, 7-(guanidiniumalkylhydroxy)-1-(aza)-2-(thio)-3-(aza)-phenthiazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenoxazin-1-yl, 7-(guanidiniumalkyl-hydroxy)-1,3-(diaza)-2-(oxo)-phenthiazin-1-yl, 7-(guanidiniumalkyl-hydroxy)-1,3-(diaza)-2-(oxo)-phenthiazin-1-yl, 7-(guanidiniumalkylhydroxy)-1,3-(diaza)-2-(oxo)-phenoxazin-1-yl, 7-(methyl)guanine, 7-(propynyl)isocarbostyrilyl, 7-(propynyl)isocarbostyrilyl, propynyl-7-(aza)indolyl, 7-allyl-8-oxoguanosine, 7-aminomethyl-7-deazaguanosine, 7-cyano-7-deazaguanosine, 7-deaza-2,6-diaminopurine, 7-Deaza-2′-amino-A/U/G/C, 7-deaza-2-amino-purine, 7-Deaza-2′-deoxy-A/U/G/C, 7-deaza-2′-deoxy-guanosine, 7-Deaza-2′-fluoro-A/U/G/C, 7-Deaza-2′-O-methyl-A/U/G/C, 7-deaza-7-substituted purine, 7-deaza-8-aza-2,6-diaminopurine, 7-deaza-8-aza-2-aminopurine, 7-deaza-8-aza-adenine, 7-deaza-2-aminopurine, 7-deaza-8-aza-adenosine, 7-deaza-8-aza-guanosine, 7-deaza-8-substituted purine, 7-Deaza-A/U/G/C, 7-deaza-adenine, 7-deaza-adenosine, 7-deaza-guanosine, 7-deaza-inosinyl, 7-methyl-8-oxo-guanosine, 7-methyladenine, 7-methylguanosine, 7-methylinosine, 7-substituted 1-(aza)-2-(thio)-3-(aza)-phenoxazin-1-yl, 7-substituted 1,3-(diaza)-2-(oxo)-phenoxazin-1-yl, 8 (alkenyl)adenine, 8 (alkyl)guanine, 8 (alkynyl)adenine, 8 (alkynyl)guanine, 8 (amino)adenine, 8 (halo)guanine, 8 (thioalkyl)adenine, 8 (thioalkyl)guanine, 8-(alkenyl)adenine, 8-(alkenyl)guanine, 8-(alkyl)adenine, 8-(alkyl)guanine, 8-(alkynyl)adenine, 8-(alkynyl)guanine, 8-(amino)adenine, 8-(amino)guanine, 8-(halo)adenine, 8-(halo)guanine, 8-(hydroxyl)adenine, 8-(hydroxyl)guanine, 8-(thioalkyl)adenine, 8-(thioalkyl)guanine, 8-(thiol)adenine, 8-(thiol)guanine, 8-Aza-2′-amino-A/U/G/C, 8-Aza-2′-deoxy-A/U/G/C, 8-Aza-2′-fluoro-A/U/G/C, 8-Aza-2′-O-methyl-A/U/G/C, 8-Aza-A/U/G/C, 8-Aza-ATP, 8-azapurine, 8-Azido-2′-amino-A/U/G/C, 8-Azido-2′-deoxy-A/U/G/C, 8-Azido-2′-fluoro-A/U/G/C, 8-Azido-2′-O-methyl-A/U/G/C, 8-Azido-A/U/G/C, 8-azido-adenosine, 8-bromo-adenosine TP, 8-bromo-guanosine TP, 8-mercapto-guanosine, 8-oxo-guanosine, 8-Trifluoromethyladenosine TP, 9-(methyl)-imidizopyridinyl, 9-Deazaadenosine TP, 9-Deazaguanosine TP, allyamino-thymidine, allyamino-uracil, Alpha-thio-pseudo-UTP, aminoindolyl, anthracenyl, archaeosine, aza adenine, aza cytosine, aza guanine, aza thymidine, aza uracil, azidotriphosphate, benzimidazole, beta-D-mannosyl-queosine, bis-ortho-(aminoalkylhydroxy)-6-phenyl-pyrrolo-pyrimidin-2-on-3-yl, bis-ortho-substituted-6-phenyl-pyrrolo-pyrimidin-2-on-3-yl, cytidine 5′-O-(1-thiophosphate), deaza adenine, deaza cytosine, deaza guanine, deaza thymidine, deaza uracil, deoxy-cytosine, deoxy-inosine, deoxyribonucleotides of C5-propynylpyrimidines, deoxyribonucleotides of diaminopurine, deoxyribonucleotides of nitropyrrole, deoxy-thymidine, difluorotolyl, dihydropseudouridine, dihydrouridine, epoxyqueuosine, Formycin A TP, Formycin B TP, galactosyl-queuosine, hydroxywybutosine, hypoxanthine, imidizopyridinyl, inosine, inosinyl, isocarbostyrilyl, isoguanisine, isopentenyladenosine, isowyosine, lysidine, mannosylqueuosine, methylphosphonates, methylphosphoramidates, methylwyosine, N (methyl)guanine, N-(methyl)guanine, N<2>-dimethylguanine, N1-methyl-adenosine, N1-methyl-guanosine, N1-methyl-pseudo-uridine, N2,7,2′-O-trimethylguanosine, N2,7-dimethylguanosine, N2,N2,7-trimethylguanosine, N2,N2-dimethyl-6-thio-guanosine, N2,N2-dimethylguanosine, N2-isobutyl-guanosine TP, N2-methyl-6-thio-guanosine, N2-methylguanosine, N2-substituted purines, N3 (methyl)uracil, N4 (acetyl)cytosine, N4,2′-O-dimethylcytidine, N4,N4-Dimethyl-2′-OMe-Cytidine TP, N4-acetyl-2′-O-methylcytidine, N4-acetylcytidine, N4-alkylcytosine, N4-alkyldeoxycytidine, N4-Amino-cytidine TP, N4-Benzoyl-cytidine TP, N4-ethylcytosine, N4-ethyldeoxycytidine, N4-methylcytidine, N6 (methyl)adenine, N6-([6-aminohexyl]carbamoylmethyl)-adenosine, N6-(19-Amino-pentaoxanonadecyl)adenosine TP, N6-(cis-hydroxyisopentenyl)adenosine, N6-(isopentyl)adenine, N6, N6 (dimethyl)adenine, N6,2′-O-dimethyladenosine, N6,N6,2′-O-trimethyladenosine, N6,N6-dimethyladenosine, N6-acetyladenosine, N6-cis-hydroxy-isopentenyl-adenosine, N6-glycinylcarbamoyladenosine, N6-hydroxynorvalylcarbamoyladenosine, N6-isopentenyladenosine, N6-methyl-2-amino-purine, N6-methyladenosine, N6-methyl-N6-threonylcarbamoyladenosine, N6-substituted purines, N6-threonylcarbamoyladenosine, N7-methylguanosine, N7-methylinosine, N7-methyl-xanthosine, N-alkylated derivative, napthalenyl, nitrobenzimidazolyl, nitroimidazolyl, nitroindazolyl, nitropyrazolyl, nubularine, N-uracil, N-uracil-5-oxyacetic acid, N-uracil-5-oxyacetic acid methyl ester, O6-methylguanosine, O6-substituted purines, O-alkylated derivative, ortho-(aminoalkylhydroxy)-6-phenyl-pyrrolo-pyrimidin-2-on-3-yl, ortho-substituted-6-phenyl-pyrrolo-pyrimidin-2-on-3-yl, Oxoformycin TP, Pseudo-UTP-1-2-ethanoic acid, para-(aminoalkylhydroxy)-6-phenyl-pyrrolo-pyrimidin-2-on-3-yl, para-substituted-6-phenyl-pyrrolo-pyrimidin-2-on-3-yl, pentacenyl, peroxywybutosine, phenanthracenyl, phenyl-A/U/G/C, propynyl-7-(aza)indolyl, pseudoisocytidine, Pseudo-iso-cytidine, pseudouracil, pseudouridine, Pseudouridine 1-(4-methylbenzenesulfonic acid) TP, Pseudouridine 1-(4-methylbenzoic acid) TP, Pseudouridine TP 1-[3-(2-ethoxy)]propionic acid, Pseudouridine TP 1-[3-{2-(2-[2-(2-ethoxy)-ethoxy]-ethoxy)-ethoxy}]propionic acid, Pseudouridine TP 1-[3-{2-(2-[2-{2(2-ethoxy)-ethoxy}-ethoxy]-ethoxy)-ethoxy}]propionic acid, Pseudouridine TP 1-[3-{2-(2-[2-ethoxy]-ethoxy)-ethoxy}]propionic acid, Pseudouridine TP 1-[3-{2-(2-ethoxy)-ethoxy}] propionic acid, Pseudouridine TP 1-methylphosphonic acid, Pseudouridine TP 1-methylphosphonic acid diethyl ester, Pseudo-UTP-N1-3-propionic acid, Pseudo-UTP-N1-4-butanoic acid, Pseudo-UTP-N1-5-pentanoic acid, Pseudo-UTP-N1-6-hexanoic acid, Pseudo-UTP-N1-7-heptanoic acid, Pseudo-UTP-N1-methyl-p-benzoic acid, Pseudo-UTP-N1-p-benzoic acid, puromycin, pyrenyl, pyridin-4-one ribonucleoside, pyridopyrimidin-3-yl, pyridopyrimidin-3-yl, 2-oxo-7-amino-pyridopyrimidin-3-yl, pyrrolo-cytidine, pyrrolo-pseudoisocytidine, pyrrolo-pyrimidin-2-on-3-yl, pyrrolopyrimidinyl, pyrrolopyrizinyl, Pyrrolosine TP, queuosine, Sp diasteriomers of ribonucleosid-5′-O-(1-thiotriphosphates), stilbenzyl, substituted 1,2,4-triazoles, substituted 7 deazapurine, tetracenyl, tubercidine, undermodified hydroxywybutosine, uridine 5-oxyacetic acid, uridine 5-oxyacetic acid methyl ester, wybutosine, wyosine, xanthine, Xanthosine-5′-TP, xylo-adenosine, zebularine, α-thio-adenosine, α-thio-cytidine, α-thio-guanosine, and/or α-thio-uridine. In the preceeding list, the abbreviation “TP” stands for triphosphate but it should be understood that the mono- and di-phosphates may also be utilized.

Polynucleotides of the present invention may comprise one or more of the modifications taught herein.

Different sugar modifications, nucleotide modifications, and/or internucleoside linkages (e.g., backbone structures) may exist at various positions in the polynucleotide. One of ordinary skill in the art will appreciate that the nucleotide analogs or other modification(s) may be located at any position(s) of a polynucleotide such that the function of the polynucleotide is not substantially decreased. A modification may also be a 5′ or 3′ terminal modification. The polynucleotide may contain from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the polynucleotide includes a modified pyrimidine or purine. In some embodiments, the pyrimidine or purine in the polynucleotide molecule may be replaced with from about 1% to about 100% of a modified uracil or modified uridine (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100% of a modified pyrimidine or purine.

In some embodiments, the polynucleotides may comprise two or more effector module component sequences which are in a pattern such as ABABAB or AABBAABBAABB or ABCABCABC or variants thereof repeated once, twice, or more than three times. In these patterns, each letter, A, B, or C represent a different effector module component.

In yet another embodiment, the polynucleotides may comprise two or more effector module component sequences with each component having one or more sequences. As a non-limiting example, the sequences may be in a pattern such as ABABAB or AABBAABBAABB or ABCABCABC or variants thereof repeated once, twice, or more than three times in each of the regions. As another non-limiting example, the sequences may be in a pattern such as ABABAB or AABBAABBAABB or ABCABCABC or variants thereof repeated once, twice, or more than three times across the entire polynucleotide. In these patterns, each letter, A, B, or C represent a different sequence or component.

### Codon Selection

In some embodiments, one or more codons of the polynucleotides of the present invention may be replaced with other codons encoding the native amino acid sequence to tune the expression of the SREs, through a process referred to as codon selection. Since mRNA codon, and tRNA anticodon pools tend to vary among organisms, cell types, sub cellular locations and over time, the codon selection described herein is a spatiotemporal (ST) codon selection.

In some embodiments of the invention, certain polynucleotide features may be codon optimized. Codon optimization refers to a process of modifying a nucleic acid sequence for enhanced expression in the host cell by replacing at least 1, 2, 3, 4, 5, 10, 15, 20, 25, 50 or more codons of the native sequence with codons that are most frequently used in the genes of that host cell while maintaining the native amino acid sequence. Codon usage may be measured using the Codon Adaptation Index (CAI) which measures the deviation of a coding polynucleotide sequence from a reference gene set. Codon usage tables are available at the Codon Usage Database (http://www.kazusa.or.jp/codon/) and the CAI can be calculated by EMBOSS CAI program (http://emboss.sourceforge.net/). Codon optimization methods are known in the art and may be useful in efforts to achieve one or more of several goals. These goals include to match codon frequencies in target and host organisms to ensure proper folding, bias nucleotide content to alter stability or reduce secondary structures, minimize tandem repeat codons or base runs that may impair gene construction or expression, customize transcriptional and translational control regions, insert or remove protein signaling sequences, remove/add post translation modification sites in encoded protein (e.g. glycosylation sites), add, remove or shuffle protein domains, insert or delete restriction sites, modify ribosome binding sites and degradation sites, to adjust translational rates to allow the various domains of the protein to fold properly, or to reduce or eliminate problem secondary structures within the polynucleotide. Codon optimization tools, algorithms and services are known in the art, and non-limiting examples include services from GeneArt (Life Technologies), DNA2.0 (Menlo Park Calif.), OptimumGene (GenScript, Piscataway, N.J.), algorithms such as but not limited to, DNAWorks v3.2.3 and/or proprietary methods. In one embodiment, a polynucleotide sequence or portion thereof is codon optimized using optimization algorithms. Codon options for each amino acid are well-known in the art as are various species table for optimizing for expression in that particular species.

In some embodiments of the invention, certain polynucleotide features may be codon optimized. For example, a preferred region for codon optimization may be upstream (5′) or downstream (3′) to a region which encodes a polypeptide. These regions may be incorporated into the polynucleotide before and/or after codon optimization of the payload encoding region or open reading frame (ORF).

After optimization (if desired), the polynucleotides components are reconstituted and transformed into a vector such as, but not limited to, plasmids, viruses, cosmids, and artificial chromosomes.

Spatiotemporal codon selection may impact the expression of the polynucleotides of the invention, since codon composition determines the rate of translation of the mRNA species and its stability. For example, tRNA anticodons to optimized codons are abundant, and thus translation may be enhanced. In contrast, tRNA anticodons to less common codons are fewer and thus translation may proceed at a slower rate. Presnyak et al. have shown that the stability of an mRNA species is dependent on the codon content, and higher stability and thus higher protein expression may be achieved by utilizing optimized codons (Presnyak et al. (2015) Cell 160, 1111-1124; the contents of which are incorporated herein by reference in their entirety). Thus, in some embodiments, ST codon selection may include the selection of optimized codons to enhance the expression of the SRES, effector modules and biocircuits of the invention. In other embodiments, spatiotemporal codon selection may involve the selection of codons that are less commonly used in the genes of the host cell to decrease the expression of the compositions of the invention. The ratio of optimized codons to codons less commonly used in the genes of the host cell may also be varied to tune expression.

In some embodiments, certain regions of the polynucleotide may be preferred for codon selection. For example, a preferred region for codon selection may be upstream (5′) or downstream (3′) to a region which encodes a polypeptide. These regions may be incorporated into the polynucleotide before and/or after codon selection of the payload encoding region or open reading frame (ORF).

The stop codon of the polynucleotides of the present invention may be modified to include sequences and motifs to alter the expression levels of the SREs, payloads and effector modules of the present invention. Such sequences may be incorporated to induce stop codon readthrough, wherein the stop codon may specify amino acids e.g. selenocysteine or pyrrolysine. In other instances, stop codons may be skipped altogether to resume translation through an alternate open reading frame. Stop codon read through may be utilized to tune the expression of components of the effector modules at a specific ratio (e.g. as dictated by the stop codon context). Examples of preferred stop codon motifs include UGAN, UAAN, and UAGN, where N is either C or U.

### Conjugates

It is contemplated by the present invention that the compositions of the present invention may be complexed, conjugated or combined with one or more homologous or heterologous molecules. As used herein, the term “homologous molecule” refers to a molecule which is similar in at least one of structure or function relative to a starting molecule while a “heterologous molecule” is one that differs in at least one of structure or function relative to a starting molecule. Structural homologs are therefore molecules which may be substantially structurally similar. In some embodiments, such homologs may be identical. Functional homologs are molecules which may be substantially functionally similar. In some embodiments, such homologs may be identical.

Pharmaceutical compositions, biocircuits, biocircuit components, effector modules including their SREs or payloads of the present invention may comprise conjugates. Such conjugates of the invention may include naturally occurring substances or ligands, such as proteins (e.g., human serum albumin (HSA), low-density lipoprotein (LDL), high-density lipoprotein (HDL), or globulin); carbohydrates (e.g., a dextran, pullulan, chitin, chitosan, inulin, cyclodextrin or hyaluronic acid); or lipids. Conjugates may also be recombinant or synthetic molecules, such as synthetic polymers, e.g., synthetic polyamino acids, an oligonucleotide (e.g. an aptamer). Examples of polyamino acids may include polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly (2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, conjugates may also include targeting groups. As used herein, the term “targeting group” refers to a functional group or moiety attached to an agent that facilitates localization of the agent to a desired region, tissue, cell and/or protein. Such targeting groups may include, but are not limited to cell or tissue targeting agents or groups (e.g. lectins, glycoproteins, lipids, proteins, an antibody that binds to a specified cell type such as a kidney cell or other cell type). In some embodiments, targeting groups may comprise thyrotropins, melanotropins, lectins, glycoproteins, surfactant protein A, mucin carbohydrates, multivalent lactose, multivalent galactose, N-acetyl-galactosamine, N-acetyl-gulucosamine, multivalent mannose, multivalent fucose, glycosylated polyaminoacids, multivalent galactose, transferrin, bisphosphonate, polyglutamate, polyaspartate, lipids, cholesterol, steroids, bile acids, folates, vitamin B12, biotin, an RGD peptide, an RGD peptide mimetic or an aptamer.

In some embodiments, targeting groups may be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also comprise hormones and/or hormone receptors.

In some embodiments, targeting groups may be any ligand capable of targeting specific receptors. Examples include, without limitation, folate, GalNAc, galactose, mannose, mannose-6-phosphate, apatamers, integrin receptor ligands, chemokine receptor ligands, transferrin, biotin, serotonin receptor ligands, PSMA, endothelin, GCPII, somatostatin, LDL, and HDL ligands. In some embodiments, targeting groups are aptamers. Such aptamers may be unmodified or comprise any combination of modifications disclosed herein.

In still other embodiments, pharmaceutical compositions, biocircuits, biocircuit components, effector modules including their SREs or payloads of the present invention may be covalently conjugated to cell penetrating polypeptides. In some embodiments, cell-penetrating peptides may also include signal sequences. In some embodiments, conjugates of the invention may be designed to have increased stability, increased cell transfection and/or altered biodistribution (e.g., targeted to specific tissues or cell types.)

In some embodiments, conjugating moieties may be added to pharmaceutical compositions, biocircuits, biocircuit components, effector modules including their SREs or payloads of the present invention such that they allow the attachment of detectable labels to targets for clearance. Such detectable labels include, but are not limited to biotin labels, ubiquitins, fluorescent molecules, human influenza hemagglutinin (HA), c-myc, histidine (His), flag, glutathione S-transferase (GST), V5 (a paramyxovirus of simian virus 5 epitope), biotin, avidin, streptavidin, horse radish peroxidase (HRP) and digoxigenin.

In some embodiments, pharmaceutical compositions, biocircuits, biocircuit components, effector modules including their SREs or payloads of the present invention may be combined with one another or other molecules in the treatment of diseases and/or conditions.

In one embodiment, the SREs or payloads of the present invention may be, but are not limited to, Payload No. 1-20419 of U.S. Patent Application Publication No. 20190192691, fragments and variants thereof.

In one embodiment, the SRE or payload of the present invention may be a conditionally active biologic protein. A wild type protein, may be used to generate a conditionally active biologic protein which are reversibly or irreversibly inactivated at the wild type normal physiological conditions as well as to such conditionally active biologic proteins and uses of such conditional active biologic proteins are provided. Such methods and conditionally active proteins are taught in, for example, International Publication No. WO2015175375 and WO2016036916 and US Patent Publication No. US20140378660, the contents of each of which are incorporated herein by reference in their entirety.

### Stimuli

Biocircuits of the present invention are triggered by one or more stimuli. Stimuli may be selected from a ligand, an externally added or endogenous metabolite, the presence or absence of a defined ligand, pH, temperature, light, ionic strength, radioactivity, cellular location, subject site, microenvironment, the presence or concentration of one or more cations or one or more anions, the presence or action of one or more effector modules, a concentration gradient of ions or biomolecules or the like, or the presence or concentration of one or more metal ions.

### Ligands

In some embodiments, the stimulus is a ligand. Ligands may be nucleic acid-based, protein-based, lipid-based, organic, inorganic or any combination of the foregoing.

In some embodiments, the ligand is selected from the group consisting of a protein, peptide, nucleic acid, lipid, lipid derivative, sterol, steroid, metabolite, metabolite derivative, and small molecule.

### Ligand Conjugates

In some embodiments, the ligand may be complexed or bound to another molecule such as for example, another ligand, a protein, peptide, nucleic acid, lipid, lipid derivative, sterol, steroid, metabolite, metabolite derivative or small molecule. In some embodiments, the ligand stimulus is complexed to or bound to one or more other molecules. In some embodiments, the ligand stimulus is complexed or bound to one or more different kinds and/or numbers of other molecules. In some embodiment, the ligand stimulus is a multimer of the same kind of ligand. In some embodiments, the ligand stimulus multimer comprises 2, 3, 4, 5, 6, or more monomers.

### Small Molecules

In some embodiments, the stimulus is a small molecule. In some embodiments, the small molecules are cell permeable.

### Cellular Location or Microenvironment

In some embodiments, the stimulus is a cellular location. In some embodiments, the cellular location is selected from the group consisting of the nucleus, the cytoplasm, a membrane, lysosome, mitochondria, endoplasmic reticulum, cellular organelle, cytoskeletal protein or subregion, intracellular membrane surface, a transmembrane region, and the extracellular matrix.

In some embodiments, the stimulus is a microenvironment or cellular niche. Microenvironments may be selected from the group consisting of a tumor microenvironment, the cell periphery, the cell membrane, the nuclear membrane, an endosome, a microenvironment characterized by an intracellular or extracellular gradient, and cytoskeletal structures or regions.

### Subject Site

In some embodiments, the stimulus is a subject site. The subject site may be selected from a location in the subject selected from the blood, plasma, an organ selected from liver, kidney, brain, heart, lung, bone, and bone marrow.

### Ion Concentration and Metals

In some embodiments, the stimuli is the presence of one or more cations and the cation is selected from the group consisting of Aluminum, Ammonium, Barium, Calcium, Chromium(II), Chromium(III), Copper(I), Copper(II), Iron(II), Iron(III), Hydrogen, Hydronium, Lead(II), Lithium, Magnesium, Manganese(II), Manganese(III), Mercury(I), Mercury(II), Nitronium, Potassium, Silver, Sodium, Strontium, Tin(II), Tin(IV), and Zinc.

In some embodiments, the stimuli is the presence of one or more anions or oxoanions selected from the group consisting of Chloride, Fluoride, Arsenate, Phosphate, Arsenite, Hydrogen phosphate, Dihydrogen phosphate, Sulfate, Nitrate, Hydrogen sulfate, Nitrite, Thiosulfate, Sulfite, Perchlorate, Iodate, Chlorate, Bromate, Chlorite, Hypochlorite, Hypobromite, Carbonate, Chromate, Hydrogen carbonate or Bicarbonate, Dichromate, Acetate, formate, Cyanide, Cyanate, Peroxide, Thiocyanate, Oxalate, Hydroxide, and Permanganate.

In some embodiments, the stimulus is the presence of one or more metal ions and the metal ion is selected from the group consisting of Magnesium, Manganese, Calcium and Zinc.

In some embodiments, stimulus is a ligand. A non-exhaustive listing of ligands which may be used as a stimulus in the present invention are described in Table 1 of U.S. Patent Application Publication No. 20190192691. Table 1 of U.S. Patent Application Publication No. 20190192691 provides the name of the ligand (Ligand Name) and the ligand number which may be referenced throughout the specification.

Design of SREs from Ligand-Ligand Binding Partner Pairs

In some embodiments, the ligand and ligand binding pairs taught in Tables 2 and 3 of U.S. Patent Application Publication No. 20190192691, may be used as the starting point or reference sequence for the design of one or more SRE's which are responsive to the ligand of the pair. Such design is taught herein and in the Examples.

Given in Tables 2 and 3 of U.S. Patent Application Publication No. 20190192691, are ligand and binding partner pairs (known targets of the ligands).

A non-exhaustive listing of sets of ligands useful in the present invention are given in Table 2 of U.S. Patent Application Publication No. 20190192691. Table 2 of U.S. Patent Application Publication No. 20190192691 provides the ligand number (as described in Table 1 of U.S. Patent Application Publication No. 20190192691), synonyms for the ligand (Ligand Synonym), the name of the target (Target Name) and the identification number of the payload (Payload ID). Additional ligand sets similar to those listed in Table 2 of U.S. Patent Application Publication No. 20190192691 and additional information regarding the ligand sets listed in Table 2 of U.S. Patent Application Publication No. 20190192691 may be found in the Drugbank database.

### Antibody Fragments and Variants

In some embodiments, antibody fragments encoded by payloads of the invention comprise antigen binding regions from intact antibodies. Examples of antibody fragments may include, but are not limited to Fab, Fab′, F(ab′)2, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking antigen. Pharmaceutical compositions, biocircuits, biocircuit components, effector modules including their SREs or payloads of the present invention may comprise one or more of these fragments. For the purposes herein, an “antibody” may comprise a heavy and light variable domain as well as an Fc region.

As used herein, the term “native antibody” refers to a usually heterotetrameric glycoprotein of about 150,000 daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia Pa. 2012. Ch. 3, p 47-54, the contents of which are herein incorporated by reference in their entirety). Determining residues making up CDRs may include the use of numbering schemes including, but not limited to, those taught by Kabat (Wu, T. T. et al., 1970, JEM, 132(2):211-50 and Johnson, G. et al., 2000, Nucleic Acids Res. 28(1): 214-8, the contents of each of which are herein incorporated by reference in their entirety), Chothia (Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety), Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

VH and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. PeerJ. 2: e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain (to form a single chain Fv (scFv)) or through the introduction of a disulfide bridge between heavy and light chain variable domains (Strohl, W. R. Therapeutic Antibody Engineering. Woodhead Publishing, Philadelphia Pa. 2012. Ch. 3, p 46-47, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

As used herein, the term “diabody” refers to a small antibody fragment with two antigen-binding sites. Diabodies comprise a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, protein phosphorylation and dephosphorylation, metabolic processes, proliferative signaling and cell division. In some embodiments, methods of the present invention may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, pharmaceutical compositions, biocircuits, biocircuit components, effector modules including their SREs or payloads of the present invention may be antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, Fynomers and Kunitz and domain peptides. In other embodiments, antibody mimetics may include one or more non-peptide regions.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

In one embodiment, the antibody is an anti-CD22 antibody. As a non-limiting example, the anti-CD22 antibody is any of the antibodies, fragments or variants thereof described in US Patent Publication No. US20150086562, the contents of which are herein incorporated by reference in their entirety, such as a heavy chain variable region having the amino acid sequences of SEQ ID NO: 49-64 in US20150086562, or a light chain variable region having the amino acid sequence of SEQ ID NO: 17-32 in US20150086562.

In one embodiment, the antibody is a recombinant monoclonal antibody derived from B cells of a non-human host which has been immunochallenged with one or more target antigens as described in US Patent Publication No. US20130281303, the contents of which are herein incorporated by reference in their entirety. As a non-limiting example, the method comprises screening B cells to generate a B cell library enriched in B cells capable of binding to the at least one target antigen, amplifying cDNA obtained from mRNA expressed in the B cell library to prepare an immunoglobulin library comprising VH and VL domains, generating antibodies from the VH and VL domains whereby the antibodies comprise light chain/heavy chain combinations and whereby the number of combinations generated is more than the number of B cells in the enriched B cell library, and screening the antibodies with the at least one target antigen to identify a subset of antibodies capable of binding to the at least one target antigen.

In one embodiment, the antibody may be a conditionally active biologic protein. An antibody, such as those described in Table 5 of U.S. Patent Application Publication No. 20190192691 may be used to generate a conditionally active biologic protein which are reversibly or irreversibly inactivated at the wild type normal physiological conditions as well as to such conditionally active biologic proteins and uses of such conditional active biologic proteins are provided. Such methods and conditionally active proteins are taught in, for example, International Publication No. WO2015175375 and WO2016036916 and US Patent Publication No. US20140378660, the contents of each of which are incorporated herein by reference in their entirety.

In one embodiment, the antibody may be a humanized full-length antibody. As a non-limiting example, the antibody may have been humanized using the methods taught in US Patent Publication No. US20130303399, the contents of which are herein incorporated by reference in their entirety.

In one embodiment, the antibody may be a humanized or CDR-grafted anti-IL6 antibody. As a non-limiting example, the anti-IL6 antibody may have been humanized using the methods taught in US Patent Publication No. US20100138945, the contents of which are herein incorporated by reference in their entirety.

In one embodiment, the antibody may comprise a modified Fc region. As a non-limiting example, the modified Fc region may be made by the methods or may be any of the regions described in US Patent Publication No. US20150065690, the contents of which are herein incorporated by reference in their entireties.

### Multispecific Antibodies

In some embodiments, payloads of the invention may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In one embodiment, the multispecific antibody may be generated and optimized by the methods described in International Patent Publication No. WO2011109726 and US Patent Publication No. US20150252119, the contents of which each of which are herein incorporated by reference in its entirety. These antibodies are able to bind to multiple antigens with high specificity and high affinity.

### Bispecific Antibodies

In some embodiments, payloads of the invention may encode bispecific antibodies. Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

In some embodiments, antibodies of the present invention may be diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs. 2010. January-February; 2(1):77-83).

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

Also included are maxibodies (bivalent scFV fused to the amino terminus of the Fc (CH2-CH3 domains) of IgG.

Third generation molecules include “miniaturized” antibodies. Among the best examples of mAb miniaturization are the small modular immunopharmaceuticals (SMIPs) from Trubion Pharmaceuticals. These molecules, which can be monovalent or bivalent, are recombinant single-chain molecules containing one VL, one VH antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE and possibly multiple sclerosis, although these projects remain in the earliest stages of clinical testing. (Nelson, A. L., MAbs. 2010. January-February; 2(1):77-83).

In some cases, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies (see, e.g., Nelson, A. L., MAbs. 2010. January-February; 2(1):77-83).

In some embodiments, payloads may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (VHHs) of heavy chain antibodies found in camels and llamas, which lack light chains (Nelson, A. L., MAbs. 2010. January-February; 2(1):77-83).

Disclosed and claimed in PCT Publication WO2014144573 to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, payloads of the invention may encode tetravalent bispecific antibodies (TetBiAbs as disclosed and claimed in PCT Publication WO2014144357). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, payloads of the invention may encode biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic VH and VL dimers, 2) VH-VL or VL-VH single chains wherein the VH and VL are attached by a polypeptide linker, or 3) individuals VH or VL domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, payloads may encode antibodies with antibody acceptor frameworks taught in U.S. Pat. No. 8,399,625. Such antibody acceptor frameworks may be particularly well suited accepting CDRs from an antibody of interest.

### Intrabodies

In some embodiments, payloads of the invention may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast growing bio-market of recombinant antibodies provides intrabodies with enhanced binding specificity, stability and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced using methods known in the art, such as those disclosed and reviewed in: (Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

In some embodiments, the polynucleotides are designed to produce one or more antibodies, or combinations of antibodies selected from the group consisting of IgA, IgG, IgM, IgE, and IgD.

The resulting antibodies expressed in a cell, tissue or organism from the polynucleotides of the present invention may have any of the following properties and may mimic the natural isotype. They may also exhibit improved properties over the native or natural isotype.

For IgG isotype-like antibodies, they may appear as the major Ig in serum, and even at greater than 75%. They may bind to macrophages, monocytes; may act as opsonin; Cross placental transport. They may exhibit complement fixation. They may exist as a monomer of two heavy and two light chains.

For IgA isotype-like antibodies, they may appear commonly in serum. They may bind PMNs and some lymphocytes with major class in secretions such as tears, saliva, colostrum, and/or mucous. They may exhibit complement fixation but only upon aggregation. They may exist in serum as a monomer and when secreted as a dimer with J chain with the secretory piece made in epithelial cells.

For IgM isotype-like antibodies, they may appear commonly in serum. They may be made by fetus and virgin B cells. They may exhibit fixation of complement; destruction of microorganisms by agglutination and/or clumping. They may exist as a pentamer or monomer where all heavy chains are identical and all light chains identical.

For IgE isotype-like antibodies, they may appear less commonly in serum. They may be involved in allergic reactions; release of mediators of allergic symptoms or play a role in parasitic helminth disease. They may exhibit no fixation of complement. They may exist as monomer with extra domain in constant region; binds to Fc receptors on basophils and mast cells before antigen interaction.

For IgD isotype-like antibodies, they may appear less commonly in serum. They may be found on the surface of B cells as a receptor for antigens. They may exhibit no fixation of complement. They may exist as a monomer with additional amino acids at C-terminus for membrane anchoring. They may associate with Ig-alpha and Ig-beta.

The polynucleotides of the present invention may be engineered to produce any standard class of immunoglobulins using an antibody described herein or any of its component parts as a starting molecule.

In one embodiment, the polynucleotides have a modular design to encode at least one of the antibodies, fragments or variants thereof. As a non-limiting example, the polynucleotide construct may encode any of the following designs: (1) the heavy chain of an antibody, (2) the light chain of an antibody, (3) the heavy and light chain of the antibody, (4) the heavy chain and light chain separated by a linker, (5) the VH1, CH1, CH2, CH3 domains, a linker and the light chain or (6) the VH1, CH1, CH2, CH3 domains, VL region, and the light chain. Any of these designs may also comprise optional linkers between any domain and/or region.

In one embodiment, the polynucleotides have a modular design and encode a polypeptide of interest such as, but not limited to, an antibody, fragment or variant thereof described herein.

Bicistronic and/or Pseudo-Bicistronic Antibody Payloads

According to the present invention, a bicistronic payload is a polynucleotide encoding a two-protein chain antibody on a single polynucleotide strand. A pseudo-bicistronic payload is a polynucleotide encoding a single chain antibody discontinuously on a single polynucleotide strand. For bicistronic payloads, the encoded two strands or two portions/regions and/or domains (as is the case with pseudo-bicistronic) are separated by at least one nucleotide not encoding the strands or domains. More often the separation comprises a cleavage signal or site or a non-coding region of nucleotides. Such cleavage sites include, for example, furin cleavage sites encoded as an “RKR” site, or a modified furin cleavage site in the resultant polypeptide or any of those taught herein.

### Single Domain Antibody Payloads

According to the present invention, a single domain payload comprises one or two polynucleotides encoding a single monomeric variable antibody domain. Typically, single domain antibodies comprise one variable domain (VH) of a heavy-chain antibody.

### Single Chain Fv Antibody Payloads

According to the present invention, a single chain Fv payloads is a polynucleotide encoding at least two coding regions and a linker region. The scFv payload may encode a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. Other linkers include those known in the art and disclosed herein.

### Bispecific Antibody Payloads

According to the present invention, a bispecific payload is a polynucleotide encoding portions or regions of two different antibodies. Bispecific payloads encode polypeptides which may bind two different antigens. Polynucleotides of the present invention may also encode trispecific antibodies having an affinity for three antigens.

### Additional Effector Module Features

### Signal Sequences

In addition to the SRE and payload region, effector modules of the invention may further comprise one or more additional features such as one or more signal sequences. Representative signal sequences are given in Table 6.

Signal sequences (sometimes referred to as signal peptides, targeting signals, target peptides, localization sequences, transit peptides, leader sequences or leader peptides) direct proteins (e.g., the effector module of the present invention) to their designated cellular and/or extracellular locations. Protein signal sequences play a central role in the targeting and translocation of nearly all secreted proteins and many integral membrane proteins.

A signal sequence is a short (5-30 amino acids long) peptide present at the N-terminus of the majority of newly synthesized proteins that are destined towards a particular location. Signal sequences can be recognized by signal recognition particles (SRPs) and cleaved using type I and type II signal peptide peptidases. Signal sequences derived from human proteins can be incorporated as a regulatory module of the effector module to direct the effector module to a particular cellular and/or extracellular location (See Table 6 of U.S. Patent Application Publication No. 20190192691). These signal sequences are experimentally verified and can be cleaved (Zhang Z. and Henzel W. J.; “Signal peptide prediction based on analysis of experimentally verified cleavage sites.”; Protein Sci. 2004, 13:2819-2824.)

In some embodiments, a signal sequence may be, although not necessarily, located at the N-terminus or C-terminus of the effector module, and may be, although not necessarily, cleaved off the desired effector module to yield a “mature” payload.

In addition to signal sequences naturally occurring such as from a secreted protein, a signal sequence may be a variant modified from a known signal sequence of a protein. For example, U.S. Pat. Nos. 8,258,102 and 9,133,265 to Sleep disclose a modified albumin signal sequence having a secretion signal and an additional X1-X2-X3-X4-X5-motif which can increase protein secretion; U.S. Pat. No. 9,279,007 to Do discloses signal sequences of modified fragments of human immunoglobulin heavy chain binding protein (Bip) that can enhance protein expression and secretion; U.S. Pat. No. 8,148,494 to Leonhartsberger et al., discloses a signal peptide with a cleavage site that can be fused with a recombinant protein; the contents of each of which are incorporated by reference in their entirety.

In some instances, the secreted signal sequences may be cytokine signal sequences such as, but not limited to, IL2 signal sequence or a p40 signal sequence.

In some instances, signal sequences directing the payload of interest to the surface membrane of the target cell may be used. Expression of the payload on the surface of the target cell may be useful to limit the diffusion of the payload to non-target in vivo environments, thereby potentially improving the safety profile of the payloads. Additionally, the membrane presentation of the payload may allow for physiologically and qualitative signaling as well as stabilization and recycling of the payload for a longer half-life. Membrane sequences may be the endogenous signal sequence of the N terminal component of the payload of interest. Optionally, it may be desirable to exchange this sequence for a different signal sequence. Signal sequences may be selected based on their compatibility with the secretory pathway of the cell type of interest so that the payload is presented on the surface of the T cell. In some embodiments, the signal sequence may be IgE signal sequence, or CD8a signal sequence.

Other signal sequence variants may be used in the present effector module may include those discussed in U.S. patent application publication NOs.: 2007/0141666; PCT patent application publication NOs.: 1993/018181; the contents of each of which are incorporated herein by reference in their entirety.

In other embodiments, a signal sequence may be a heterogeneous signal sequence from other organisms such as virus, yeast and bacteria, which can direct an effector module to a particular cellular site, such as a nucleus (e.g., EP 1209450). Other examples may include Aspartic Protease (NSP24) signal sequences from Trichoderma that can increase secretion of fused protein such as enzymes (e.g., U.S. Pat. No. 8,093,016 to Cervin and Kim), bacterial lipoprotein signal sequences (e.g., PCT application publication NO.: 1991/09952 to Lau and Rioux), E. coli enterotoxin II signal peptides (e.g., U.S. Pat. No. 6,605,697 to Kwon et al.), E. coli secretion signal sequence (e.g., U.S. patent publication NO.: 2016/090404 to Malley et al.), a lipase signal sequence from a methylotrophic yeast (e.g., U.S. Pat. No. 8,975,041), and signal peptides for DNases derived from Coryneform bacteria (e.g., U.S. Pat. No. 4,965,197); the contents of each of which are incorporated herein by reference in their entirety.

Signal sequences may also include nuclear localization signals (NLSs), nuclear export signals (NESs), polarized cell tubulo-vesicular structure localization signals (See, e.g., U.S. Pat. No. 8,993,742; Cour et al., Nucleic Acids Res. 2003, 31(1): 393-396; the contents of each of which are incorporated herein by reference in their entirety), extracellular localization signals, signals to subcellular locations (e.g. lysosome, endoplasmic reticulum, golgi, mitochondria, plasma membrane and peroxisomes, etc.) (See, e.g., U.S. Pat. No. 7,396,811; Negi et al., Database, 2015, 1-7; the contents of each of which are incorporated herein by reference in their entirety.)

### Construct Optimization to Reduce Basal Expression

Biocircuit constructs are to be further optimized to reduce or eliminate the basal expression in the absence of ligands. In some embodiments, an interfering RNA may be used to reduce the basal expression. Other RNA regulatory elements may also be introduced to the construct, for example, by incorporating AU-rich mRNA destabilizing elements (ARE) into the 3′ untranslated region (3′UTR) of the construct (Maitra et al., RNA, 2008, 14(5): 950-959).

In some embodiments, a construct may be test with different promoters or mutated promoters. The promoter that gives the least “leaky” expression may be used. In some embodiments, one or more suppressor binding sites may be inserted to the constructs. The suppressor proteins bind to the construct and suppress the expression of the construct in the absence of the stimulus.

Additionally, constructs encoding proteins which can attenuate the transgene activity may also be co-expressed with the biocircuits of the present invention.

In some embodiments, effector modules of the present invention may include one or more degrons to tune expression. As used herein, a “degron” refers to a minimal sequence within a protein that is sufficient for the recognition and the degradation by the proteolytic system. An important property of degrons is that they are transferrable, that is, appending a degron to a sequence confers degradation upon the sequence. In some embodiments, the degron may be appended to the destabilizing domains, the payload or both. Incorporation of the degron within the effector module of the invention, confers additional protein instability to the effector module and may be used to minimize basal expression. In some embodiments, the degron may be an N-degron, a phospho degron, a heat inducible degron, a photosensitive degron, an oxygen dependent degron. As a non-limiting example, the degron may be an Ornithine decarboxylase degron as described by Takeuchi et al. (Takeuchi J et al. (2008). Biochem J. 2008 Mar. 1; 410(2):401-7; the contents of which are incorporated by reference in their entirety). Other examples of degrons useful in the present invention include degrons described in International patent publication Nos. WO2017004022, WO2016210343, and WO2011062962; the contents of each of which are incorporated by reference in their entirety.

### III. Pharmaceutical Compositions

The present teachings further comprise pharmaceutical compositions comprising one or more of the stimuli, biocircuits, effector modules or systems of the present invention, and optionally at least one pharmaceutically acceptable excipient or inert ingredient.

As used herein the term “pharmaceutical composition” refers to a preparation of one or more of the biocircuits or components described herein, or pharmaceutically acceptable salts thereof, optionally with other chemical components such as physiologically suitable carriers and excipients.

The term “excipient” or “inactive ingredient” refers to an inert or inactive substance added to a pharmaceutical composition to further facilitate administration of a compound. Non-limiting examples of such inert ingredients are disclosed herein under Formulations.

In some embodiments, compositions are administered to humans, human patients or subjects. For the purposes of the present disclosure, the phrase “active ingredient” generally refers to any one or more biocircuit system component to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient or inert ingredient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of compositions of the present invention, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present invention can also be judged using an experimental animal model for the given disease as known in the art. When using an experimental animal model, efficacy of treatment is evidenced when a statistically significant change is observed.

### Therapeutic Uses

### Cancer

Various cancers may be treated with pharmaceutical compositions, biocircuits, biocircuit components, effector modules including their SREs or payloads of the present invention. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present invention include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of carcinomas which may be treated with the compositions of the present invention include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

### Combination Treatments

The invention further relates to the use of pharmaceutical compositions, biocircuits, biocircuit components, effector modules including their SREs or payloads of the present invention for treating one or more forms of cancer, in combination with other pharmaceuticals and/or other therapeutic methods, e.g., with known pharmaceuticals and/or known therapeutic methods, such as, for example, those which are currently employed for treating these disorders. For example, the pharmaceutical compositions, biocircuits, biocircuit components, effector modules including their SREs or payloads of the present invention can also be administered in conjunction with one or more additional anti-cancer treatments, such as biological, chemotherapy and radiotherapy. Accordingly, a treatment can include, for example, imatinib (Gleevac), all-trans-retinoic acid, a monoclonal antibody treatment (gemtuzumab, ozogamicin), chemotherapy (for example, chlorambucil, prednisone, prednisolone, vincristine, cytarabine, clofarabine, farnesyl transferase inhibitors, decitabine, inhibitors of MDR1), rituximab, interferon-α, anthracycline drugs (such as daunorubicin or idarubicin), L-asparaginase, doxorubicin, cyclophosphamide, doxorubicin, bleomycin, fludarabine, etoposide, pentostatin, or cladribine), bone marrow transplant, stem cell transplant, radiation therapy, anti-metabolite drugs (methotrexate and 6-mercaptopurine), or any of the antibodies taught herein such as those in Table 5 of U.S. Patent Application Publication No. 20190192691 or combinations thereof.

Combinations with Radiation

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. The effects of radiation therapy are localized and confined to the region being treated. Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma.

Combination with Chemotherapy

Chemotherapy is the treatment of cancer with drugs that can destroy cancer cells. In current usage, the term “chemotherapy” usually refers to cytotoxic drugs which affect rapidly dividing cells in general, in contrast with targeted therapy. Chemotherapy drugs interfere with cell division in various possible ways, e.g. with the duplication of DNA or the separation of newly formed chromosomes. Most forms of chemotherapy target all rapidly dividing cells and are not specific to cancer cells, although some degree of specificity may come from the inability of many cancer cells to repair DNA damage, while normal cells generally can.

Most chemotherapy regimens are given in combination. Exemplary chemotherapeutic agents include, but are not limited to, 5-FU Enhancer, 9-AC, AG2037, AG3340, Aggrecanase Inhibitor, Aminoglutethimide, Amsacrine (m-AMSA), Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorombucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p′-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL®), PARP Inhibitors, PD 183805, Pentostatin (2′ deoxycoformycin), PKC 412, Plicamycin, Procarbazine HCl, PSC 833, Ralitrexed, RAS Farnesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/Uracil), Valrubicin, Vinblastine sulfate, Vindesine sulfate, VX-710, VX-853, YM 116, ZD 0101, ZD 0473/Anormed, ZD 1839, ZD 9331.

### Immuno-Oncology and Cell Therapies

Recent progress in the field of cancer immunology has allowed the development of several approaches to help the immune system keep the cancer at bay. Such immunotherapy approaches include the targeting of cancer antigens through monoclonal antibodies or through adoptive transfer of ex vivo engineered T cells (e.g., which contain chimeric antigen receptors or engineered T cell receptors).

In some embodiments, pharmaceutical compositions, biocircuits, biocircuit components, effector modules including their SREs or payloads of the present invention may be used in the modulation or alteration or exploitation of the immune system to target one or more cancers. This approach may also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the pharmaceutical compositions, biocircuits, biocircuit components, effector modules including their SREs or payloads of the present invention.

Cancer immunotherapy refers to a diverse set of therapeutic strategies designed to induce the patient's own immune system to fight the cancer. In some embodiments, pharmaceutical compositions, biocircuits, biocircuit components, effector modules including their SREs or payloads of the present invention are designed as immune-oncology therapeutics.

### Cell Therapies

There are several types of cellular immunotherapies, including tumor infiltrating lymphocyte (TIL) therapy, genetically engineered T cells bearing chimeric antigen receptors (CARs), and recombinant TCR technology.

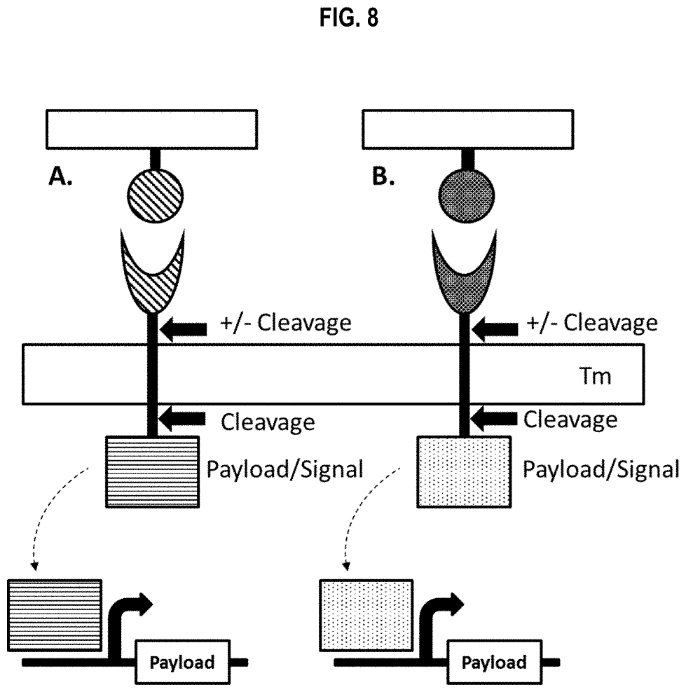

According to the present invention, the biocircuits and systems may be used in the development and implementation of cell therapies such as adoptive cell therapy. Certain effector modules useful in cell therapy are given in FIGS. 8-13. The biocircuits, their components, effector modules and their SREs and payloads may be used in cell therapies to effect TCR removal—TCR gene disruption, TCR engineering, to regulate epitope tagged receptors, in APC platforms for stimulating T cells, as a tool to enhance ex vivo APC stimulation, to improve methods of T cell expansion, in ex vivo stimulation with antigen, in TCR/CAR combinations, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g by introducing cytokine genes, genes for the checkpoint inhibitors PD1, CTLA4).

Components of the biocircuits useful in cell therapies include for example, those in Tables 1-3.

TABLE 1
T-Cell Receptor (heterodimer)

| Symbol | Name |
|---|---|
| TRA (TCRA) | T cell receptor alpha chain |
| TRAV | T cell receptor alpha chain variable region |
| TRAJ | T cell receptor alpha chain joining region |
| TRAC | T cell receptor alpha chain constant region |
| TRB (TCRB) | T cell receptor beta chain |
| TRBV | T cell receptor beta chain variable region |
| TRBD | T cell receptor beta chain diversity region |
| TRBJ | T cell receptor beta chain joining region |
| TRBC | T cell receptor beta chain constant region |
| TRD (TCRD) | T cell receptor delta chain |
| TRDV | T cell receptor delta chain variable region |
| TRDD | T cell receptor delta chain diversity region |
| TRDJ | T cell receptor delta chain joining region |
| TRDC | T cell receptor delta chain constant region |
| TRG (TCRG) | T cell receptor gamma chain |
| TRGV (variable) gene | T cell receptor gamma chain variable |
| | region |
| TRGJ (joining) gene | T cell receptor gamma chain joining |
| | region |
| TRGC (constant) gene | T cell receptor gamma chain constant |
| | region |

TABLE 2
T-Cell Co-Receptor

| Symbol | Name | NCBI Ref No. |
|---|---|---|
| CD4 | CD4 Molecule | NP_000607 |
| CD8 | T-cell surface glycoprotein CD8 alpha | NP_001139345.1 |
| | chain isoform 1 precursor | (GI: 225007536) |
| CD8 | T-cell surface glycoprotein CD8 alpha | NP_741969.1 |
| | chain isoform 2 precursor | (GI: 27886642) |
| CD8 | T-cell surface glycoprotein CD8 beta | NP_001171571.1 |
| | chain isoform 6 precursor | (GI: 296010933) |
| CD8 | T-cell surface glycoprotein CD8 beta | NP_757362.1 |
| | chain isoform 2 precursor | (GI: 27886639) |
| CD8 | T-cell surface glycoprotein CD8 beta | NP_742100.1 |
| | chain isoform 4 precursor | (GI: 27886637) |
| CD8 | T-cell surface glycoprotein CD8 beta | NP_742099.1 |
| | chain isoform 3 precursor | (GI: 27886635) |
| CD8 | T-cell surface glycoprotein CD8 beta | NP_004922.1 |
| | chain isoform 5 precursor | (GI: 4826667) |

TABLE 3
T-Cell Receptor Signaling Transduction Complex

| Complex | Name | NCBI Ref No. |
|---|---|---|
| CD3 | CD3 delta chain | NP_000723 |
| CD3 | CD3 gamma chain | NP_000064 |
| CD3 | CD3 epsilon chain | NP_000724 |
| CD3 | CD 3 zeta chain | NP_000725 |
| Other | CD3 Zeta-chain-associated protein kinase | NP_001070 |
| | 70 (ZAP70) | |
| Other | Proto-oncogene tyrosine-protein kinase | NP_002028 |
| | (FYN) | |

In some embodiments, improved response rates are obtained in support of cell therapies.

Expansion and persistence of cell populations may be achieved through regulation or fine tuning of the payloads, e.g., the receptors or pathway components in T cells, NK cells or other immune-related cells. In some embodiments, biocircuits, their components, SREs or effector modules are designed to spatially and/or temporally control the expression of proteins which enhance T-cell or NK cell response. In some embodiments, biocircuits, their components, SREs or effector modules are designed to spatially and/or temporally control the expression of proteins which inhibit T-cell or NK cell response.

In some embodiments, biocircuits, their components, SREs or effector modules are designed to reshape the tumor microenvironment to extend utility of the biocircuit or a pharmaceutical composition beyond direct cell killing.

In some embodiments, biocircuits, their components, SREs or effector modules are designed to reduce, mitigate or eliminate the CAR cytokine storm. In some embodiments, such reduction, mitigation and/or elimination occurs in solid tumors or tumor microenvironments.

In some embodiments, the effector modules may encode one or more cytokines.

In one embodiment, the payload of the invention may comprise IL2. In one aspect, the effector module of the invention may be DD-IL2 fusion polypeptide. The amino acid sequences corresponding to DD-IL2 and its components are listed in Table 4. In Table 4, the mutations in the sequences are underlined.

TABLE 4
DD-IL2 constructs

| | | Amino |
|---|---|---|
| | | Acid |
| | | SEQ |
| | | ID |
| Description | Amino Acid Sequence | NO |
| IL2 signal | MYRMQLLSCIALSLALVTNS | 1 |
| sequence | | |
| Linker | EFSTEF | 2 |
| IL2 | APTSSSTKKTQLQLEHLLLDLQMILNGINNYKNP | 3 |
| | KLTRMLTFKFYMPKKATELKHLQCLEEELKPLE | |
| | EVLNLAQSKNFHLRPRDLISNINVIVLELKGSETT | |
| | FMCEYADETATIVEFLNRWITFCQSIISTLT | |
| FKBP | GVQVETISPGDGRTFPKRGQTCVVHYTGMLEDG | 4 |
| (F36V, | KKVDSSRDRNKPFKFMLGKQEVIRGWEEGVAQ | |
| L106P) | MSVGQRAKLTISPDYAYGATGHPGIIPPHATLVF | |
| | DVELLKPE | |
| ecDHFR | ISLIAALAVDYVIGMENAMPWNLPADLAWFKR | 5 |
| (R12Y, | NTLNKPVIMGRHTWESIGRPLPGRKNIILSSQPGT | |
| Y100I) | DDRVTWVKSVDEAIAACGDVPEIMVIGGGRVIE | |
| | QFLPKAQKLYLTHIDAEVEGDTHFPDYEPDDWE | |
| | SVFSEFHDADAQNSHSYCFEILERR | |
| OT-IL2-001 | MYRMQLLSCIALSLALVTNSEFSTEFGVQVETI | 6 |
| (IL2 signal | SPGDGRTFPKRGQTCVVHYTGMLEDGKKVDSSR | |
| sequence; | DRNKPFKFMLGKQEVIRGWEEGVAQMSVGQRA | |
| linker1 | KLTISPDYAYGATGHPGIIPPHATLVFDVELLKP | |
| (EFSTEF); | EMHAPTSSSTKKTQLQLEHLLLDLQMILNGINNY | |
| FKBP | KNPKLTRMLTFKFYMPKKATELKHLQCLEEELKP | |
| (F36V, | LEEVLNLAQSKNFHLRPRDLISNINVIVLELKGS | |
| L106P); | ETTFMCEYADETATIVEFLNRWITFCQSIISTLT | |
| linker2 | | |
| (MH); | | |
| IL2) | | |
| OT-IL2-002 | MYRMQLLSCIALSLALVTNSAPTSSSTKKTQLQL | 7 |
| (Signal | EHLLLDLQMILNGINNYKNPKLTRMLTFKFYMP | |
| sequence- | KKATELKHLQCLEEELKPLEEVLNLAQSKNFHL | |
| IL2) | RPRDLISNINVIVLELKGSETTFMCEYADETAT | |
| | IVEFLNRWITFCQSIISTLT | |
| OT-IL2-003 | MYRMQLLSCIALSLALVTNSEFSTEFISLIAALA | 8 |
| (IL2 signal | VDYVIGMENAMPWNLPADLAWFKRNTLNKPVIM | |
| sequence; | GRHTWESIGRPLPGRKNIILSSQPGTDDRVTWVK | |
| linker1 | SVDEAIAACGDVPEIMVIGGGRVIEQFLPKAQKL | |
| (EFSTEF); | YLTHIDAEVEGDTHFPDYEPDDWESVFSEFHDA | |
| ecDHFR | DAQNSHSYCFEILERRMHAPTSSSTKKTQLQLEH | |
| (R12Y, | LLLDLQMILNGINNYKNPKLTRMLTFKFYMPKK | |
| Y100I); | ATELKHLQCLEEELKPLEEVLNLAQSKNFHLRPR | |
| linker2 | DLISNINVIVLELKGSETTFMCEYADETATIVEF | |
| (MH); | LNRWITFCQSIISTLT | |
| IL2 | | |

In one aspect, the effector module of the invention may be a DD-IL12 fusion polypeptide. Regulatable DD-IL12 fusion polypeptide may be directly used as an immunotherapeutic agent or be transduced into an immune effector cell (T cells and TIL, cells) to generate modified T cells with greater in vivo expansion and survival capabilities for adoptive cell transfer. The need for harsh preconditioning regimens in current adoptive cell therapies may be minimized using regulated IL12 DD-IL12 may be utilized to modify tumor microenvironment and increase persistence in solid tumors that are currently refractory to tumor antigen targeted therapy. In some embodiments, CAR expressing T cells may be armored with DD regulated IL12 to relieve immunosuppression without systemic toxicity.

In some embodiments, the IL12 may be a Flexi IL12, wherein both p35 and p40 subunits, are encoded by a single cDNA that produces a single chain polypeptide. In some aspects, the DD-IL12 comprises the amino acid sequences as shown in Table 5. In Table 5, the mutations in the sequences are underlined.

TABLE 5
DD-IL12 constructs

| | | Amino |
|---|---|---|
| | | Acid |
| Description/ | | SEQ ID |
| Construct ID | Amino Acid Sequence | NO |
| p40 signal sequence | MCHQQLVISWFSLVFLASPLVA | 9 |
| Linker | GGSGG | 10 |
| Linker | GGGGSGGGGSGGGGS | 11 |
| Furin cleavage site | SARNRQKRS | 12 |
| Furin cleavage site | ARNRQKRS | 13 |
| Modified Furin | ESRRVRRNKRSK | 14 |
| p40 | IWELKKDVYVVELDWYPDAPGEMVVLTCDTPEEDGITWT | 15 |
| | LDQSSEVLGSGKTLTIQVKEFGDAGQYTCHKGGEVLSHSL | |
| | LLLHKKEDGIWSTDILKDQKEPKNKTFLRCEAKNYSGRFT | |
| | CWWLTTISTDLTFSVKSSRGSSDPQGVTCGAATLSAERVR | |
| | GDNKEYEYSVECQEDSACPAAEESLPIEVMVDAVHKLKYE | |
| | NYTSSFFIRDIIKPDPPKNLQLKPLKNSRQVEVSWEYPDT | |
| | WSTPHSYFSLTFCVQVQGKSKREKKDRVFTDKTSATVICR | |
| | KNASISVRAQDRYYSSSWSEWASVPCS | |
| p35 | RNLPVATPDPGMFPCLHHSQNLLRAVSNMLQKARQTLEFY | 16 |
| | PCTSEEIDHEDITKDKTSTVEACLPLELTKNESCLNSRETS | |
| | FITNGSCLASRKTSFMMALCLSSIYEDLKMYQVEFKTMNA | |
| | KLLMDPKRQIFLDQNMLAVIDELMQALNFNSETVPQKSSL | |
| | EEPDFYKTKIKLCILLHAFRIRAVTIDRVMSYLNAS | |
| FKBP (F36V, | GVQVETISPGDGRTFPKRGQTCVVHYTGMLEDGKKVDSS | 4 |
| L106P) | RDRNKPFKFMLGKQEVIRGWEEGVAQMSVGQRAKLTISP | |
| | DYAYGATGHPGIIPPHATLVFDVELLKPE | |
| FKBP (E31G, F36V, | GVQVETISPGDGRTFPKRGQTCVVHYTGMLGDGKKVDSS | 17 |
| R71G, K105E) | RDRNKPFKFMLGKQEVIRGWEEGVAQMSVGQGAKLTISP | |
| | DYAYGATGHPGIIPPHATLVFDVELLELE | |
| ecDHFR (R12Y, | ISLIAALAVDYVIGMENAMPWNLPADLAWFKRNTLNKPVI | 5 |
| Y100I) | MGRHTWESIGRPLPGRKNIILSSQPGTDDRVTWVKSVDEAI | |
| | AACGDVPEIMVIGGGRVIEQFLPKAQKLYLTHIDAEVEGD | |
| | THFPDYEPDDWESVFSEFHDADAQNSHSYCFEILERR | |
| OT-IL12-001 (p40 | MCHQQLVISWFSLVFLASPLVAGVQVETISPGDGRTFPKR | 18 |
| signal sequence; | GQTCVVHYTGMLEDGKKVDSSRDRNKPFKFMLGKQEVIR | |
| FKBP (F36V, | GWEEGVAQMSVGQRAKLTISPDYAYGATGHPGIIPPHATL | |
| L106P); linker1 | VFDVELLKPEGGSGGIWELKKDVYVVELDWYPDAPGEMV | |
| (GGSGG); p40; | VLTCDTPEEDGITWTLDQSSEVLGSGKTLTIQVKEFGDAG | |
| linker2 (G4S)3; p35) | QYTCHKGGEVLSHSLLLLHKKEDGIWSTDILKDQKEPKNK | |
| | TFLRCEAKNYSGRFTCWWLTTISTDLTFSVKSSRGSSDPQG | |
| | VTCGAATLSAERVRGDNKEYEYSVECQEDSACPAAEESLP | |
| | IEVMVDAVHKLKYENYTSSFFIRDIIKPDPPKNLQLKPLK | |
| | NSRQVEVSWEYPDTWSTPHSYFSLTFCVQVQGKSKREKKD | |
| | RVFTDKTSATVICRKNASISVRAQDRYYSSSWSEWASVPC | |
| | SGGGGSGGGGSGGGGSRNLPVATPDPGMFPCLHHSQNLLR | |
| | AVSNMLQKARQTLEFYPCTSEEIDHEDITKDKTSTVEACL | |
| | PLELTKNESCLNSRETSFITNGSCLASRKTSFMMALCLSS | |
| | IYEDLKMYQVEFKTMNAKLLMDPKRQIFLDQNMLAVIDEL | |
| | MQALNFNSETVPQKSSLEEPDFYKTKIKLCILLHAFRIRA | |
| | VTIDRVMSYLNAS | |
| OT-IL12-002 (FKBP | MGVQVETISPGDGRTFPKRGQTCVVHYTGMLEDGKKVDS | 19 |
| (F36V, L106P); | SRDRNKPFKFMLGKQEVIRGWEEGVAQMSVGQRAKLTIS | |
| linker1 (GGSGG); | PDYAYGATGHPGIIPPHATLVFDVELLKPEGGSGGMCHQ | |
| p40 signal sequence; | QLVISWFSLVFLASPLVAIWELKKDVYVVELDWYPDAPGE | |
| p40; linker | MVVLTCDTPEEDGITWTLDQSSEVLGSGKTLTIQVKEFGD | |
| 2((G4S)3); p35) | AGQYTCHKGGEVLSHSLLLLHKKEDGIWSTDILKDQKEPK | |
| | NKTFLRCEAKNYSGRFTCWWLTTISTDLTFSVKSSRGSSD | |
| | PQGVTCGAATLSAERVRGDNKEYEYSVECQEDSACPAAEE | |
| | SLPIEVMVDAVHKLKYENYTSSFFIRDIIKPDPPKNLQLK | |
| | PLKNSRQVEVSWEYPDTWSTPHSYFSLTFCVQVQGKSKRE | |
| | KKDRVFTDKTSATVICRKNASISVRAQDRYYSSSWSEWAS | |
| | VPCSGGGGSGGGGSGGGGSRNLPVATPDPGMFPCLHHSQN | |
| | LLRAVSNMLQKARQTLEFYPCTSEEIDHEDITKDKTSTVE | |
| | ACLPLELTKNESCLNSRETSFITNGSCLASRKTSFMMALC | |
| | LSSIYEDLKMYQVEFKTMNAKLLMDPKRQIFLDQNMLAVI | |
| | DELMQALNFNSETVPQKSSLEEPDFYKTKIKLCILLHAFR | |
| | IRAVTIDRVMSYLNAS | |
| OT-IL12-003 (p40 | MCHQQLVISWFSLVFLASPLVAGVQVETISPGDGRTFPKR | 20 |
| signal sequence; | GQTCVVHYTGMLEDGKKVDSSRDRNKPFKFMLGKQEVIR | |
| FKBP(F36V, | GWEEGVAQMSVGQRAKLTISPDYAYGATGHPGIIPPHATL | |
| L106P); furin | VFDVELLKPESARNRQKRSIWELKKDVYVVELDWYPDAP | |
| (SARNRQKRS); | GEMVVLTCDTPEEDGITWTLDQSSEVLGSGKTLTIQVKEF | |
| p40; linker | GDAGQYTCHKGGEVLSHSLLLLHKKEDGIWSTDILKDQKE | |
| ((G4S)3); | PKNKTFLRCEAKNYSGRFTCWWLTTISTDLTFSVKSSRGSS | |
| p35) | DPQGVTCGAATLSAERVRGDNKEYEYSVECQEDSACPAA | |
| | EESLPIEVMVDAVHKLKYENYTSSFFIRDIIKPDPPKNLQLK | |
| | PLKNSRQVEVSWEYPDTWSTPHSYFSLTFCVQVQGKSKRE | |
| | KKDRVFTDKTSATVICRKNASISVRAQDRYYSSSWSEWAS | |
| | VPCSGGGGSGGGGSGGGGSRNLPVATPDPGMFPCLHHSQ | |
| | NLLRAVSNMLQKARQTLEFYPCTSEEIDHEDITKDKTSTVE | |
| | ACLPLELTKNESCLNSRETSFITNGSCLASRKTSFMMALCL | |
| | SSIYEDLKMYQVEFKTMNAKLLMDPKRQIFLDQNMLAVI | |
| | DELMQALNFNSETVPQKSSLEEPDFYKTKIKLCILLHAFRIR | |
| | AVTIDRVMSYLNAS | |
| OT-IL12-004 | MCHQQLVISWFSLVFLASPLVAIWELKKDVYVVELDWYP | 21 |
| (p40 signal | DAPGEMVVLTCDTPEEDGITWTLDQSSEVLGSGKTLTIQV | |
| sequence; p40; | KEFGDAGQYTCHKGGEVLSHSLLLLHKKEDGIWSTDILKD | |
| linker ((G4S)3); | QKEPKNKTFLRCEAKNYSGRFTCWWLTTISTDLTFSVKSS | |
| p35; furin | RGSSDPQGVTCGAATLSAERVRGDNKEYEYSVECQEDSA | |
| (SARNRQKRS); | CPAAEESLPIEVMVDAVHKLKYENYTSSFFIRDIIKPDPPKN | |
| FKBP (E31G, | LQLKPLKNSRQVEVSWEYPDTWSTPHSYFSLTFCVQVQGK | |
| F36V, R71G, | SKREKKDRVFTDKTSATVICRKNASISVRAQDRYYSSSWS | |
| K105E)) | EWASVPCSGGGGSGGGGSGGGGSRNLPVATPDPGMFPCL | |
| | HHSQNLLRAVSNMLQKARQTLEFYPCTSEEIDHEDITKDK | |
| | TSTVEACLPLELTKNESCLNSRETSFITNGSCLASRKTSFM | |
| | MALCLSSIYEDLKMYQVEFKTMNAKLLMDPKRQIFLDQN | |
| | MLAVIDELMQALNFNSETVPQKSSLEEPDFYKTKIKLCILL | |
| | HAFRIRAVTIDRVMSYLNASARNRQKRSGVQVETISPGDG | |
| | RTFPKRGQTCVVHYTGMLGDGKKVDSSRDRNKPFKFMLG | |
| | KQEVIRGWEEGVAQMSVGQGAKLTISPDYAYGATGHPGII | |
| | PPHATLVFDVELLELE | |
| OT-IL12-005 | MCHQQLVISWFSLVFLASPLVAIWELKKDVYVVELDWYP | 22 |
| (p40 signal | DAPGEMVVLTCDTPEEDGITWTLDQSSEVLGSGKTLTIQV | |
| sequence; p40; | KEFGDAGQYTCHKGGEVLSHSLLLLHKKEDGIWSTDILKD | |
| linker1 | QKEPKNKTFLRCEAKNYSGRFTCWWLTTISTDLTFSVKSS | |
| ((G4S)3); p35; | RGSSDPQGVTCGAATLSAERVRGDNKEYEYSVECQEDSA | |
| linker 2(GGSG); | CPAAEESLPIEVMVDAVHKLKYENYTSSFFIRDIIKPDPPKN | |
| FKBP (E31G, | LQLKPLKNSRQVEVSWEYPDTWSTPHSYFSLTFCVQVQGK | |
| F36V, R71G, | SKREKKDRVFTDKTSATVICRKNASISVRAQDRYYSSSWS | |
| K105E)) | EWASVPCSGGGGSGGGGSGGGGSRNLPVATPDPGMFPCL | |
| | HHSQNLLRAVSNMLQKARQTLEFYPCTSEEIDHEDITKDK | |
| | TSTVEACLPLELTKNESCLNSRETSFITNGSCLASRKTSFM | |
| | MALCLSSIYEDLKMYQVEFKTMNAKLLMDPKRQIFLDQN | |
| | MLAVIDELMQALNFNSETVPQKSSLEEPDFYKTKIKLCILL | |
| | HAFRIRAVTIDRVMSYLNASGGSGGVQVETISPGDGRTFPK | |
| | RGQTCVVHYTGMLGDGKKVDSSRDRNKPFKFMLGKQEVI | |
| | RGWEEGVAQMSVGQGAKLTISPDYAYGATGHPGIIPPHAT | |
| | LVFDVELLELE | |
| OT-IL12-006 | MCHQQLVISWFSLVFLASPLVAIWELKKDVYVVELDWYP | 23 |
| (p40 signal | DAPGEMVVLTCDTPEEDGITWTLDQSSEVLGSGKTLTIQV | |
| sequence; p40; | KEFGDAGQYTCHKGGEVLSHSLLLLHKKEDGIWSTDILKD | |
| linker((G4S)3); | QKEPKNKTFLRCEAKNYSGRFTCWWLTTISTDLTFSVKSS | |
| p35) | RGSSDPQGVTCGAATLSAERVRGDNKEYEYSVECQEDSA | |
| | CPAAEESLPIEVMVDAVHKLKYENYTSSFFIRDIIKPDPPKN | |
| | LQLKPLKNSRQVEVSWEYPDTWSTPHSYFSLTFCVQVQGK | |
| | SKREKKDRVFTDKTSATVICRKNASISVRAQDRYYSSSWS | |
| | EWASVPCSGGGGSGGGGSGGGGSRNLPVATPDPGMFPCL | |
| | HHSQNLLRAVSNMLQKARQTLEFYPCTSEEIDHEDITKDK | |
| | TSTVEACLPLELTKNESCLNSRETSFITNGSCLASRKTSFM | |
| | MALCLSSIYEDLKMYQVEFKTMNAKLLMDPKRQIFLDQN | |
| | MLAVIDELMQALNFNSETVPQKSSLEEPDFYKTKIKLCILL | |
| | HAFRIRAVTIDRVMSYLNAS | |
| OT-IL12-007 | MCHQQLVISWFSLVFLASPLVAISLIAALAVDYVIGMENA | 24 |
| (p40 signal | MPWNLPADLAWFKRNTLNKPVIMGRHTWESIGRPLPGRK | |
| sequence; | NIILSSQPGTDDRVTWVKSVDEAIAACGDVPEIMVIGGGRV | |
| ecDHFR (R12Y, | IEQFLPKAQKLYLTHIDAEVEGDTHFPDYEPDDWESVFSEF | |
| Y100I); furin site | HDADAQNSHSYCFEILERRESRRVRRNKRSKIWELKKDVY | |
| (ESRRVRRNK | VVELDWYPDAPGEMVVLTCDTPEEDGITWTLDQSSEVLG | |
| RSK); p40; | SGKTLTIQVKEFGDAGQYTCHKGGEVLSHSLLLLHKKEDG | |
| linker((G4S)3); | IWSTDILKDQKEPKNKTFLRCEAKNYSGRFTCWWLTTIST | |
| p35) | DLTFSVKSSRGSSDPQGVTCGAATLSAERVRGDNKEYEYS | |
| | VECQEDSACPAAEESLPIEVMVDAVHKLKYENYTSSFFIRD | |
| | IIKPDPPKNLQLKPLKNSRQVEVSWEYPDTWSTPHSYFSLT | |
| | FCVQVQGKSKREKKDRVFTDKTSATVICRKNASISVRAQD | |
| | RYYSSSWSEWASVPCSGGGGSGGGGSGGGGSRNLPVATP | |
| | DPGMFPCLHHSQNLLRAVSNMLQKARQTLEFYPCTSEEID | |
| | HEDITKDKTSTVEACLPLELTKNESCLNSRETSFITNGSCL | |
| | ASRKTSFMMALCLSSIYEDLKMYQVEFKTMNAKLLMDPKR | |
| | QIFLDQNMLAVIDELMQALNFNSETVPQKSSLEEPDFYKT | |
| | KIKLCILLHAFRIRAVTIDRVMSYLNAS | |
| OT-IL12-009 (p40 | MCHQQLVISWFSLVFLASPLVAIWELKKDVYVVELDWYP | 25 |
| signal sequence; p40; | DAPGEMVVLTCDTPEEDGITWTLDQSSEVLGSGKTLTIQV | |
| linker((G4S)3); p35; | KEFGDAGQYTCHKGGEVLSHSLLLLHKKEDGIWSTDILKD | |
| furin | QKEPKNKTFLRCEAKNYSGRFTCWWLTTISTDLTFSVKSS | |
| (ESRRVRRNKRSK); | RGSSDPQGVTCGAATLSAERVRGDNKEYEYSVECQEDSA | |
| FKBP (E31G, | CPAAEESLPIEVMVDAVHKLKYENYTSSFFIRDIIKPDPPKN | |
| F36V, R71G, | LQLKPLKNSRQVEVSWEYPDTWSTPHSYFSLTFCVQVQGK | |
| K105E)) | SKREKKDRVFTDKTSATVICRKNASISVRAQDRYYSSSWS | |
| | EWASVPCSGGGGSGGGGSGGGGSRNLPVATPDPGMFPCL | |
| | HHSQNLLRAVSNMLQKARQTLEFYPCTSEEIDHEDITKDK | |
| | TSTVEACLPLELTKNESCLNSRETSFITNGSCLASRKTSFM | |
| | MALCLSSIYEDLKMYQVEFKTMNAKLLMDPKRQIFLDQN | |
| | MLAVIDELMQALNFNSETVPQKSSLEEPDFYKTKIKLCILL | |
| | HAFRIRAVTIDRVMSYLNASESRRVRRNKRSKGVQVETISP | |
| | GDGRTFPKRGQTCVVHYTGMLGDGKKVDSSRDRNKPFKF | |
| | MLGKQEVIRGWEEGVAQMSVGQGAKLTISPDYAYGATG | |
| | HPGIIPPHATLVFDVELLELE | |

In some embodiments, effector modules may encode, or be tuned or induced to produce, one or more cytokines for expansion of cells in the biocircuits of the invention. In such cases the cells may be tested for actual expansion. Expansion may be at least 5%, 10%, 20%, 30%, 40%, 50%, 60%, 70%, 80%, 90% or more. In some embodiments, the cytokine is IL-15. Effector modules encoding IL-15 may be designed to induce proliferation in cytotoxic populations and avoid stimulation of T regs. In other cases, the effector modules which induce proliferation in cytotoxic populations may also stimulate NK and NKT cells. Interleukin 15 is a potent immune stimulatory cytokine and an essential survival factor for T cells, and Natural Killer cells. Preclinical studies comparing IL2 and IL15, have shown than IL15 is associated with less toxicity than IL-2. In some embodiments, the effector module of the invention may be a DD-IL15 fusion polypeptide. IL15 polypeptide may also be modified to increase its binding affinity for the IL15 receptor. For example, the asparagine may be replaced by aspartic acid at position 72 of IL15 (SEQ ID NO. 2 of US patent publication US20140134128; the contents of which are incorporated by reference in their entirety). In some aspects, the DD-IL15 comprises the amino acid sequences listed in Table 6. In Table 6, the mutations in the sequences are in bold.

TABLE 6
DD IL15 constructs

| | | Amino |
|---|---|---|
| | | Acid |
| | | SEQ |
| | | ID |
| Description | Amino acid sequence | NO |
| IL2 signal | MYRMQLLSCIALSLALVTNS | 1 |
| sequence | | |
| Linker | EFSTEF | 2 |
| Linker | GGSGG | 10 |
| IL15 | NWVNVISDLKKIEDLIQSMHIDAT | 26 |
| | LYTESDVHPSCKVTAMKCFLLELQ | |
| | VISLESGDASIHDTVENLIILANN | |
| | SLSSNGNVTESGCKECEELEEKNI | |
| | KEFLQSFVHIVQMFINTS | |
| ecDHFR (R12Y, | ISLIAALAVDYVIGMENAMPWNL | 5 |
| Y100I) | PADLAWFKRNTLNKPVIMGRHTW | |
| | ESIGRPLPGRKNIILSSQPGTDDR | |
| | VTWVKSVDEAIAACGDVPEIMVIG | |
| | GGRVIEQFLPKAQKLYLTHIDAE | |
| | VEGDTHFPDYEPDDWESVFSEFH | |
| | DADAQNSHSYCFEILERR | |
| OT-IL15-001 (IL2 | MYRMQLLSCIALSLALVTNSNWVN | 27 |
| signal sequence; | VISDLKKIEDLIQSMHIDATLYTE | |
| IL15) | SDVHPSCKVTAMKCFLLELQVISL | |
| | ESGDASIHDTVENLIILANNSLSSN | |
| | GNVTESGCKECEELEEKNIKEFLQ | |
| | SFVHIVQMFINTS | |
| OT-IL15-002 (IL2 | MYRMQLLSCIALSLALVTNSEFST | 28 |
| signal sequence; | EFISLIAALAVDYVIGMENAMPW | |
| linker1 [EFSTEF]; | NLPADLAWFKRNTLNKPVIMGRH | |
| ecDHFR (R12Y, | TWESIGRPLPGRKNIILSSQPGTDD | |
| Y100I); linker2 | RVTWVKSVDEAIAACGDVPEIMV | |
| [GGSGG]; IL15) | IGGGRVIEQFLPKAQKLYLTHIDA | |
| | EVEGDTHFPDYEPDDWESVFSEF | |
| | HDADAQNSHSYCFEILERRGGSG | |
| | GNWVNVISDLKKIEDLIQSMHIDA | |
| | TLYTESDVHPSCKVTAMKCFLLE | |
| | LQVISLESGDASIHDTVENLIILAN | |
| | NSLSSNGNVTESGCKECEELEEKN | |
| | IKEFLQSFVHIVQMFINTS | |

In some embodiments, the effector modules of the present invention used for the expansion of cells may include a payload comprising any of the genes of the Ras superfamily including, but not limited to, RAS such as KRAS, NRAS, RRAS, RRAS2, MRAS, ERAS, and HRAS, DIRAS such as DIRAS1, DIRAS2, and DIRAS3, NKIRAS such as NKIRAS1, and NKIRAS2, RAL such as RALA, and RALB, RAP such as RAP1A, RAP1B, RAP2A, RAP2B, and RAP2C, RASD such as RASD1, and RASD2, RASL such as RASL10A, RASL10B, RASL11A, RASL11B, and RASL12, REM such as REM1, and REM2, GEM, RERG, RERGL and RRAD.

In some embodiments, the tumor microenvironment may be remodeled using a biocircuit containing an effector module encoding IL-17.

The immune system can be harnessed for the treatment of diseases beyond cancer. Biocircuits, their components, SREs or effector modules may be utilized in immunotherapy for the treatment of diseases including, but not limited to, autoimmune diseases, allergies, graft versus host disease, and diseases and disorders that may result in immunodeficiency such as acquired immune deficiency syndrome (AIDS).

In some embodiments, payloads of the present invention may be a chimeric antigen receptor (CAR), which when transduced into immune cells (e.g., T cells and NK cells), can re-direct the immune cells against the target (e.g., a tumor cell) which expresses a molecule recognized by the extracellular target moiety of the CAR.

As used herein, the term “chimeric antigen receptor (CAR)” refers to a synthetic receptor that mimics the TCR on the surface of T cells. In general, a CAR is composed of an extracellular targeting domain, a transmembrane domain/region and an intracellular signaling/activation domain. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR complex (e.g., the signal region of CD3ζ), and/or one or more costimulatory signaling domains, such as those from CD28, 4-1BB (CD137) and OX-40 (CD134). For example, a “first-generation CAR” only has the CD3ζ signaling domain, whereas, a second-generation CARs has a CD3ζ signal domain plus one costimulatory signaling domain, and a third-generation CARs having CD3ζ signal domain plus two or more costimulatory signaling domains. A CAR, when expressed by a T cell, endows the T cell with antigen specificity determined by the extracellular targeting moiety of the CAR. It is also desirable to add one or more elements such as homing and suicide genes to develop a more competent and safer architecture of CAR, which has given rise to the so called the fourth-generation CAR.

In some embodiments, the extracellular targeting domain is joined through the hinge (also called space domain or spacer) and transmembrane regions to an intracellular signaling domain. The hinge may need to be varied to optimize the potency of CAR expressing cells towards the cancer cells due to the size of the target protein where the targeting moiety binds, and the size and affinity of the targeting domain itself. Upon recognition and binding of the targeting moiety to the target cell, the intracellular signaling domain leads to an activation signal for the CAR T cell, which is further amplified by the “second signal” from one or more intracellular costimulatory domains. The CAR T cell, once activated, can destroy the target cell.

In some embodiments, the CAR of the present invention may be split into two parts, each part is linked a dimerizing domain, such that an input that triggers the dimerization promotes assembly of the intact functional receptor. Wu and Lim recently reported a split CAR in which the extracellular CD19 binding domain and the intracellular signaling element are separated and linked to the FKBP domain and the FRB* (T2089L mutant of FKBP-rapamycin binding) domain that heterodimerize in the presence of the rapamycin analog AP21967. The split receptor is assembled in the presence of AP21967 and together with the specific antigen binding, activates T cells (Wu et al., Science, 2015, 625(6258): aab4077).

In some embodiments, the CAR of the present invention may be designed as an inducible CAR. Sakemura et al recently reported the incorporation of a Tet-On inducible system to the CD19 CAR construct. The CD19 CAR is activated only in the presence of doxycycline (Dox). Sakemura reported that Tet-CD19CAR T cells in the presence of Dox were cytotoxic against CD19+ cell lines and had equivalent cytokine production and proliferation upon CD19 stimulation, compared with conventional CD19CAR T cells (Sakemura et al., Cancer Immuno. Res., 2016, Jun. 21, Epub ahead of print). In one example, this Tet-CAR may be the payload of the effector module under the control of SREs (e.g., DDs) of the invention. The dual systems provide more flexibility to turn-on and off the CAR expression in transduced T cells.

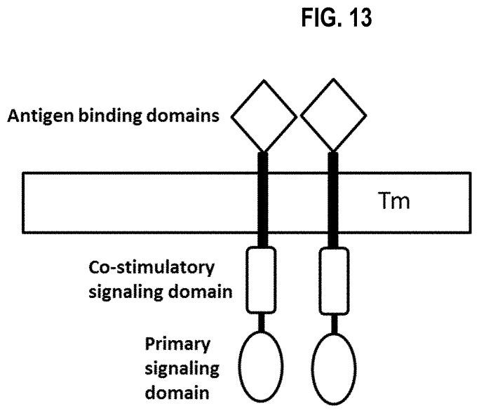

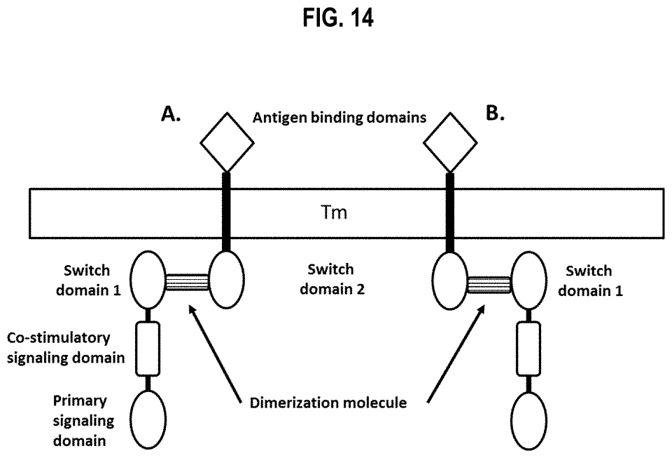

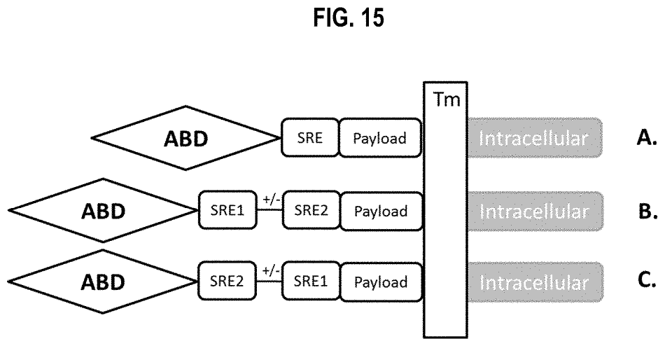

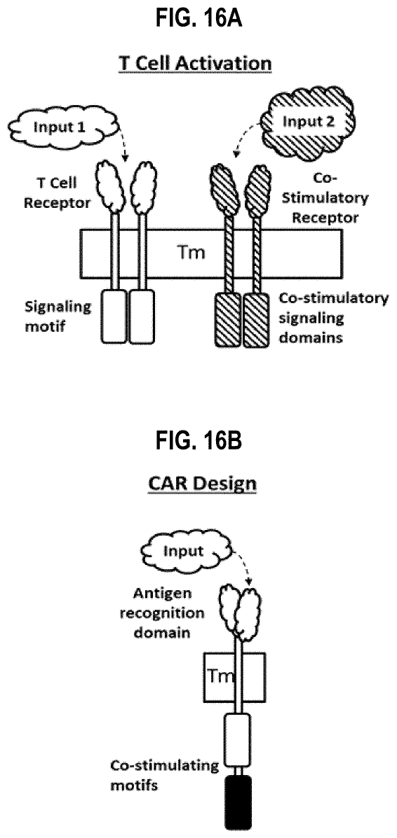

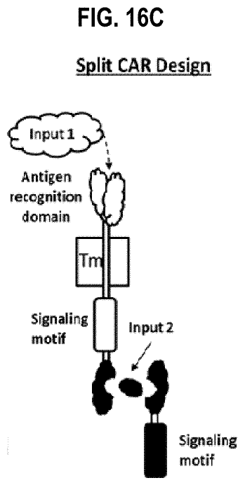

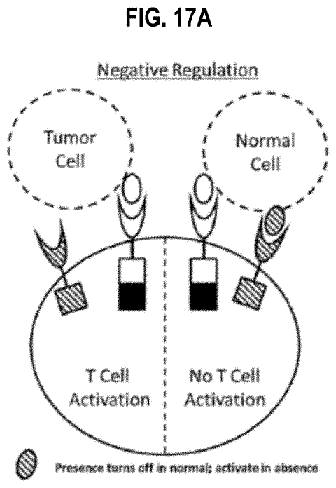

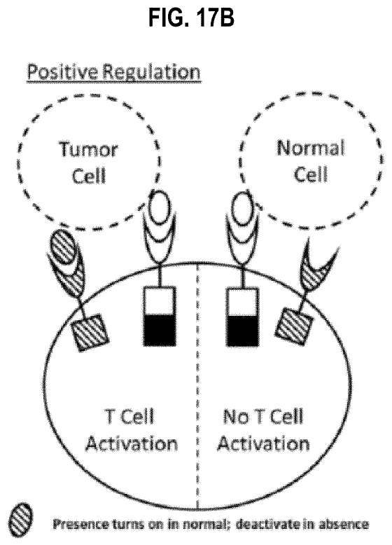

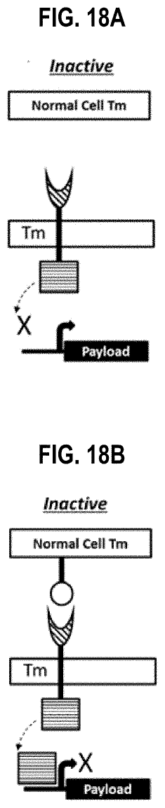

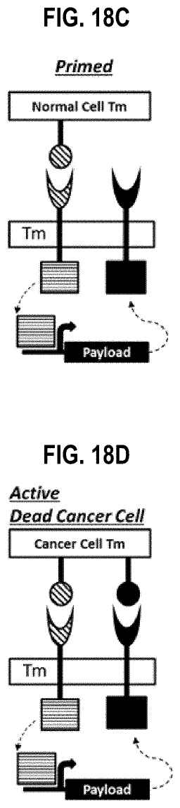

According to the present invention, the payload of the present invention may be a first-generation CAR, or a second-generation CAR, or a third-generation CAR, or a fourth-generation CAR. Representative effector module embodiments comprising CAR constructs are illustrated in FIG. 13-18.

In accordance with the invention, the extracellular target moiety of a CAR may be any agent that recognizes and binds to a given target molecule, for example, a neoantigen on tumor cells, with high specificity and affinity. The target moiety may be an antibody and variants thereof that specifically bind to a target molecule on tumor cells, or a peptide aptamer selected from a random sequence pool based on its ability to bind to the target molecule on tumor cells, or a variant or fragment thereof that can bind to the target molecule on tumor cells, or an antigen recognition domain from native T-cell receptor (TCR) (e.g. CD4 extracellular domain to recognize HIV infected cells), or exotic recognition components such as a linked cytokine that leads to recognition of target cells bearing the cytokine receptor, or a natural ligand of a receptor.

In some embodiments, the targeting domain of a CAR may be a Ig NAR, a Fab fragment, a Fab′ fragment, a F(ab)′2 fragment, a F(ab)′3 fragment, Fv, a single chain variable fragment (scFv), a bis-scFv, a (scFv)2, a minibody, a diabody, a triabody, a tetrabody, a disulfide stabilized Fv protein (dsFv), a unitbody, a nanobody, or an antigen binding region derived from an antibody that specifically recognizes a target molecule, for example a tumor specific antigen (TSA). In one embodiment, the targeting moiety is a scFv antibody. The scFv domain, when it is expressed on the surface of a CAR T cell and subsequently binds to a target protein on a cancer cell, is able to maintain the CAR T cell in proximity to the cancer cell and to trigger the activation of the T cell. A scFv can be generated using routine recombinant DNA technology techniques and is discussed in the present invention.

In some embodiments, the targeting moiety of a CAR construct may be a natural ligand of the target molecule, or a variant and/or fragment thereof capable of binding the target molecule. In some aspects, the targeting moiety of a CAR may be a receptor of the target molecule, for example, a full length human CD27, as a CD70 receptor, may be fused in frame to the signaling domain of CD3ζ forming a CD27 chimeric receptor as an immunotherapeutic agent for CD70-positive malignancies (see, e.g., US patent publication NO.: US20130323214; the contents of which are incorporated by reference herein in their entirety).

In some embodiments, the targeting moiety of a CAR may recognize a tumor specific antigen (TSA), for example a cancer neoantigen whose expression is restricted to tumor cells.

As non-limiting examples, the CAR of the present invention may comprise the extracellular targeting domain capable of binding to a tumor specific antigen selected from 5T4, 707-AP, A33, AFP (α-fetoprotein), AKAP-4 (A kinase anchor protein 4), ALK, α5β1-integrin, androgen receptor, annexin II, alpha-actinin-4, ART-4, B1, B7H3, B7H4, BAGE (B melanoma antigen), BCMA, BCR-ABL fusion protein, beta-catenin, BKT-antigen, BTAA, CA-I (carbonic anhydrase I), CA50 (cancer antigen 50), CA125, CA15-3, CA195, CA242, calretinin, CAIX (carbonic anhydrase), CAMEL (cytotoxic T-lymphocyte recognized antigen on melanoma), CAM43, CAP-1, Caspase-8/m, CD4, CD5, CD7, CD19, CD20, CD22, CD23, CD25, CD27/m, CD28, CD30, CD33, CD34, CD36, CD38, CD40/CD154, CD41, CD44v6, CD44v7/8, CD45, CD49f, CD56, CD68\KP1, CD74, CD79a/CD79b, CD103, CD123, CD133, CD138, CD171, cdc27/m, CDK4 (cyclin dependent kinase 4), CDKN2A, CDS, CEA (carcinoembryonic antigen), CEACAM5, CEACAM6, chromogranin, c-Met, c-Myc, coa-1, CSAp, CT7, CT10, cyclophilin B, cyclin B1, cytoplasmic tyrosine kinases, cytokeratin, DAM-10, DAM-6, dek-can fusion protein, desmin, DEPDC1 (DEP domain containing 1), E2A-PRL, EBNA, EGF-R (epidermal growth factor receptor), EGP-1 (epithelial glycoprotein-1) (TROP-2), EGP-2, EGP-40, EGFR (epidermal growth factor receptor), EGFRvIII, EF-2, ELF2M, EMMPRIN, EpCAM (epithelial cell adhesion molecule), EphA2, Epstein Barr virus antigens, Erb (ErbB1; ErbB3; ErbB4), ETA (epithelial tumor antigen), ETV6-AML1 fusion protein, FAP (fibroblast activation protein), FBP (folate-binding protein), FGF-5, folate receptor a, FOS related antigen 1, fucosyl GM1, G250, GAGE (GAGE-1; GAGE-2), galactin, GD2 (ganglioside), GD3, GFAP (glial fibrillary acidic protein), GM2 (oncofetal antigen-immunogenic-1; OFA-I-1), GnT-V, Gp100, H4-RET, HAGE (helicase antigen), HER-2/neu, HIFs (hypoxia inducible factors), HIF-1α, HIF-2α, HLA-A2, HLA-A*0201-R170I, HLA-Al l, HMWMAA, Hom/Mel-40, HSP70-2M (Heat shock protein 70), HST-2, HTgp-175, hTERT (or hTRT), human papillomavirus-E6/human papillomavirus-E7 and E6, iCE (immune-capture EIA), IGF-1R, IGH-IGK, IL-2R, IL-5, ILK (integrin-linked kinase), IMP3 (insulin-like growth factor II mRNA-binding protein 3), IRF4 (interferon regulatory factor 4), KDR (kinase insert domain receptor), KIAA0205, KRAB-zinc finger protein (KID)-3; KID31, KSA (17-1A), K-ras, LAGE, LCK, LDLR/FUT (LDLR-fucosyltransferaseAS fusion protein), LeY (Lewis Y), MAD-CT-1, MAGE (tyrosinase, melanoma-associated antigen) (MAGE-1; MAGE-3), melan-A tumor antigen (MART), MART-2/Ski, MC1R (melanocortin 1 receptor), MDM2, mesothelin, MPHOSPH1, MSA (muscle-specific actin), mTOR (mammalian targets of rapamycin), MUC-1, MUC-2, MUM-1 (melanoma associated antigen (mutated) 1), MUM-2, MUM-3, Myosin/m, MYL-RAR, NA88-A, N-acetylglucosaminyltransferase, neo-PAP, NF-KB (nuclear factor-kappa B), neurofilament, NSE (neuron-specific enolase), Notch receptors, NuMa, N-Ras, NY-BR-1, NY-CO-1, NY-ESO-1, Oncostatin M, OS-9, OY-TES1, p53 mutants, p190 minor bcr-abl, pl5(58), pl85erbB2, pl80erbB-3, PAGE (prostate associated gene), PAP (prostatic acid phosphatase), PAX3, PAX5, PDGFR (platelet derived growth factor receptor), cytochrome P450 involved in piperidine and pyrrolidine utilization (PIPA), Pml-RAR alpha fusion protein, PR-3 (proteinase 3), PSA (prostate specific antigen), PSM, PSMA (Prostate stem cell antigen), PRAME (preferentially expressed antigen of melanoma), PTPRK, RAGE (renal tumor antigen), Raf (A-Raf, B-Raf and C-Raf), Ras, receptor tyrosine kinases, RCAS1, RGSS, ROR1 (receptor tyrosine kinase-like orphan receptor 1), RU1, RU2, SAGE, SART-1, SART-3, SCP-1, SDCCAG16, SP-17 (sperm protein 17), src-family, SSX (synovial sarcoma X breakpoint)-1, SSX-2 (HOM-MEL-40), SSX-3, SSX-4, SSX-5, STAT-3, STAT-5, STAT-6, STEAD, STn, survivin, syk-ZAP70, TA-90 (Mac-2 binding protein\cyclophilin C-associated protein), TAAL6, TACSTD1 (tumor associated calcium signal transducer 1), TACSTD2, TAG-72-4, TAGE, TARP (T cell receptor gamma alternate reading frame protein), TEL/AML1 fusion protein, TEM1, TEM8 (endosialin or CD248), TGFβ, TIE2, TLP, TMPRSS2 ETS fusion gene, TNF-receptor (TNF-α receptor, TNF-β receptor; or TNF-γ receptor), transferrin receptor, TPS, TRP-1 (tyrosine related protein 1), TRP-2, TRP-2/INT2, TSP-180, VEGF receptor, WNT, WT-1 (Wilm's tumor antigen) and XAGE.

As non-limiting examples, the targeting moiety of the present invention may be a scFv antibody that recognizes a tumor specific antigen (TSA), for example scFvs of antibodies SS, SS1 and HN1 that specifically recognize and bind to human mesothelin (U.S. Pat. No. 9,359,447), scFv of antibody of GD2 (U.S. Pat. No. 9,315,585), a CD19 antigen binding domain (U.S. Pat. No. 9,328,156); a NKG2D ligand binding domain (U.S. Pat. No. 9,273,283; US patent publication NO.: US20160311906A1); human anti-mesothelin scFvs comprising the amino acid sequences of SEQ ID NO.: 11 and 12 of U.S. Pat. No. 9,272,002; an anti-CS1 binding agent (US patent publication NO.: US20160075784); an anti-BCMA binding domain (International Patent Publication NO.: WO2016/014565); anti-CD19 scFv antibody of SEQ ID NO.: 20 in U.S. Pat. No. 9,102,761; GFR alpha 4 antigen binding fragments having the amino acid sequences of SEQ ID NOs.: 59 and 79 of International patent publication NO.: 2016/025880; anti-CLL-1 (C-type lectin-like molecule 1) binding domains having the amino acid sequences of SEQ ID NO.:47, 44, 48, 49, 50, 39, 40, 41, 42, 43, 45, 46, 51, 73, 70, 74, 75, 76, 65, 66, 67, 68, 69, 71, 72, 77, 195, 86, 83, 87, 88, 89, 78, 79, 80, 81, 82, 84, 85, 90 and 196 of International Patent Publication NO.: WO2016014535); CD33 binding domains having the amino acid sequences of SEQ ID NOs.: 39-46 of International patent publication NO.: WO2016014576; a GPC3 (glypican-3) binding domain (SEQ ID NO.: 2 and SEQ ID NO.: 4 of International patent publication NO.: WO2016036973); a GFR alpha4 (Glycosyl-phosphatidylinositol (GPI)-linked GDNF family α-receptor 4 cell-surface receptor) binding domain (International Patent Publication NO.: WO2016025880); CD123 binding domains having the amino acid sequences of SEQ ID NOs.: 480, 483, 485, 478, 158, 159, 160, 157, 217, 218, 219, 216, 276, 277, 278, and 275 of International patent publication NO.: WO20160258896; an anti-ROR1 antibody or fragments thereof (International patent publication NO.: WO2016016344); scFvs specific to GPC-3 (SEQ ID NOs.: 1 and 24 of International patent publication NO.: WO2016049459); scFv for CSPG4 (SEQ ID NO.: 2 of International patent publication NO.: WO2015080981; scFv for folate receptor alpha (US Patent Publication NO.: US20170002072A1); the contents of each of which are incorporated herein by reference in their entirety.

The intracellular domain of a CAR fusion polypeptide, after binding to its target molecule, transmits a signal to the immune effector cell, activating at least one of the normal effector functions of immune effector cells, including cytolytic activity (e.g., cytokine secretion) or helper activity. Therefore, the intracellular domain comprises an “intracellular signaling domain” of a T cell receptor (TCR). In some embodiments, the intracellular signaling domain of the present invention may contain signaling motifs which are known as immunoreceptor tyrosine-based activation motifs (ITAMs). In some embodiments, the intracellular region of the present invention further comprises one or more costimulatory signaling domains which provide additional signals to the immune effector cells. These costimulatory signaling domains, in combination with the signaling domain can further improve expansion, activation, memory, persistence, and tumor-eradicating efficiency of CAR engineered immune cells (e.g., CAR T cells). In some cases, the costimulatory signaling region contains 1, 2, 3, or 4 cytoplasmic domains of one or more intracellular signaling and/or costimulatory molecules. In some embodiments, the intracellular region of the present invention may comprise a functional signaling domain from a protein selected from the group consisting of an MHC class I molecule, a TNF receptor protein, an immunoglobulin-like protein, a cytokine receptor, an integrin, a signaling lymphocytic activation protein (SLAM) such as CD48, CD229, 2B4, CD84, NTB-A, CRACC, BLAME, CD2F-10, SLAMF6, SLAMF7, an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), SLAMF7, NKp80 (KLRF1), NKp44, NKp30, NKp46, CD19, CD4, CD8alpha, CD8beta, IL2R beta, IL2R gamma, IL7R alpha, IL-15Ra, ITGA4, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, NKD2C SLP76, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, CD270 (HVEM), GADS, SLP-76, PAG/Cbp, CD19a, a ligand that specifically binds with CD83, DAP 10, TRIM, ZAP70, Killer immunoglobulin receptors (KIRs) such as KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and KIR2DP1; lectin related NK cell receptors such as Ly49, Ly49A, and Ly49C.

In some embodiments, the CAR of the present invention may comprise a transmembrane domain. As used herein, the term “Transmembrane domain (TM)” refers broadly to an amino acid sequence of about 15 residues in length which spans the plasma membrane. More preferably, a transmembrane domain includes at least 20, 21, 22, 23, 24, 25, 26, 27, 28, 29, 30, 31, 32, 33, 34, 35, 36, 37, 38, 39, 40, 41, 42, 43, 44, or 45 amino acid residues and spans the plasma membrane. In some embodiments, the transmembrane domain of the present invention may be derived either from a natural or from a synthetic source. The transmembrane domain of a CAR may be derived from any naturally membrane-bound or transmembrane protein. In some embodiments, the transmembrane domain of the present invention may be selected from the group consisting of a CD8α transmembrane domain, a CD4 transmembrane domain, a CD28 transmembrane domain, a CTLA-4 transmembrane domain, a PD-1 transmembrane domain, and a human IgG4 Fc region.

In some embodiments, the CAR of the present invention may comprise an optional hinge region (also called spacer). A hinge sequence is a short sequence of amino acids that facilitates flexibility of the extracellular targeting domain that moves the target binding domain away from the effector cell surface to enable proper cell/cell contact, target binding and effector cell activation (Patel et al., Gene Therapy, 1999; 6: 412-419). The hinge sequence may be positioned between the targeting moiety and the transmembrane domain. The hinge sequence can be any suitable sequence derived or obtained from any suitable molecule. The hinge sequence may be derived from all or part of an immunoglobulin (e.g., IgGl, IgG2, IgG3, IgG4) hinge region, i.e., the sequence that falls between the CHI and CH2 domains of an immunoglobulin, e.g., an IgG4 Fc hinge, the extracellular regions of type 1 membrane proteins such as CD8α CD4, CD28 and CD7, which may be a wild type sequence or a derivative.

In some embodiments, the CAR of the present invention may comprise one or more linkers selected from Table 11 or 12 of U.S. Patent Application Publication No. 20190192691.

In one embodiment of the present invention, the CAR of the present invention is a CD19 specific CAR. In the context of the invention, an effector module may comprise an ecDHFR DD or FKBP DD operably linked to a CD19 CAR fusion construct. The amino acid sequences of CD19 CAR and its components are presented in Table 7. In Table 7, the transmembrane domain is italicized and underlined to differentiate it from the adjacent sequence components. The mutations in the sequences are in bold.

TABLE 7
CD19 CAR constructs

| | | Amino |
|---|---|---|
| | | Acid |
| | | SEQ |
| | | ID |
| Description | Amino Acid Sequence | NO |
| CD19 scFv | DIQMTQTTSSLSASLGDRVTISCRASQDISK | 29 |
| | YLNWYQQKPDGTVKLLIYHTSRLHSGVPS | |
| | RFSGSGSGTDYSLTISNLEQEDIATYFCQQG | |
| | NTLPYTFGGGTKLEITGGGGSGGGGSGGG | |
| | GSEVKLQESGPGLVAPSQSLSVTCTVSGVS | |
| | LPDYGVSWIRQPPRKGLEWLGVIWGSETT | |
| | YYNSALKSRLTIIKDNSKSQVFLKMNSLQT | |
| | DDTAIYYCAKHYYYGGSYAMDYWGQGTS | |
| | VTVSS | |
| CD8α hinge-TM | TTTPAPRPPTPAPTIASQPLSLRPEACRPAAG | 30 |
| | GAVHTRGLDFACDIYIWAPLAGTCGVLLLSL | |
| | VITLYC | |
| CD3 zeta | RVKFSRSADAPAYKQGQNQLYNELNLGRR | 31 |
| signaling | EEYDVLDKRRGRDPEMGGKPRRKNPQEGL | |
| domain | YNELQKDKMAEAYSEIGMKGERRRGKGH | |
| | DGLYQGLSTATKDTYDALHMQALPPR | |
| 4-1BB | KRGRKKLLYIFKQPFMRPVQTTQEEDGCS | 32 |
| intracellular | CRFPEEEEGGCEL | |
| signaling | | |
| domain | | |
| CD8α leader | MALPVTALLLPLALLLHAARP | 33 |
| FKBP (F36V, | GVQVETISPGDGRTFPKRGQTCVVHYTGM | 4 |
| L106P) | LEDGKKVDSSRDRNKPFKFMLGKQEVIRG | |
| | WEEGVAQMSVGQRAKLTISPDYAYGATG | |
| | HPGIIPPHATLVFDVELLKPE | |
| FKBP (E31G, | GVQVETISPGDGRTFPKRGQTCVVHYTGM | 17 |
| F36V, R71G, | LGDGKKVDSSRDRNKPFKFMLGKQEVIRG | |
| K105E) | WEEGVAQMSVGQGAKLTISPDYAYGATG | |
| | HPGIIPPHATLVFDVELLELE | |
| ecDHFR (R12Y, | ISLIAALAVDYVIGMENAMPWNLPADLAW | 5 |
| Y100I) | FKRNTLNKPVIMGRHTWESIGRPLPGRKNII | |
| | LSSQPGTDDRVTWVKSVDEAIAACGDVPEI | |
| | MVIGGGRVIEQFLPKAQKLYLTHIDAEVEG | |
| | DTHFPDYEPDDWESVFSEFHDADAQNSHS | |
| | YCFEILERR | |
| ecDHFR (R12H, | ISLIAALAVDHVIGMENAMPWNLPADLAW | 34 |
| E129K) | FKRNTLNKPVIMGRHTWESIGRPLPGRKNII | |
| | LSSQPGTDDRVTWVKSVDEAIAACGDVPEI | |
| | MVIGGGRVYEQFLPKAQKLYLTHIDAEVE | |
| | GDTHFPDYKPDDWESVFSEFHDADAQNSH | |
| | SYCFEILERR | |
| Linker | GGSGG | 10 |
| OT-CD19 CAR- | MALPVTALLLPLALLLHAARPDIQMTQTTS | 35 |
| 001 (CD8a | SLSASLGDRVTISCRASQDISKYLNWYQQK | |
| leader; CD19 | PDGTVKLLIYHTSRLHSGVPSRFSGSGSGTD | |
| scFV (FM63); | YSLTISNLEQEDIATYFCQQGNTLPYTFGGG | |
| CD8a hinge + TM; | TKLEITGGGGSGGGGSGGGGSEVKLQESGP | |
| 41BB; CD3zeta) | GLVAPSQSLSVTCTVSGVSLPDYGVSWIRQ | |
| | PPRKGLEWLGVIWGSETTYYNSALKSRLTI | |
| | IKDNSKSQVFLKMNSLQTDDTAIYYCAKHY | |
| | YYGGSYAMDYWGQGTSVTVSSTTTPAPRP | |
| | PTPAPTIASQPLSLRPEACRPAAGGAVHTRG | |
| | LDFACDIYIWAPLAGTCGVLLLSLVITLYC | |
| | KRGRKKLLYIFKQPFMRPVQTTQEEDGCSC | |
| | RFPEEEEGGCELRVKFSRSADAPAYKQGQN | |
| | LYNELNLGRREEYDVLDKRRGRDPEMGGK | |
| | QPRRKNPQEGLYNELQKDKMAEAYSEIGMK | |
| | GERRRGKGHDGLYQGLSTATKDTYDALH | |
| | MQALPPR | |
| OT-CD19 CAR- | MALPVTALLLPLALLLHAARPDIQMTQTTS | 36 |
| 002 (CD8a | SLSASLGDRVTISCRASQDISKYLNWYQQK | |
| leader; CD19 | PDGTVKLLIYHTSRLHSGVPSRFSGSGSGTD | |
| scFV; FKBP | YSLTISNLEQEDIATYFCQQGNTLPYTFGGG | |
| (F36V, L106P); | TKLEITGGGGSGGGGSGGGGSEVKLQESGP | |
| CD8a hinge + TM; | GLVAPSQSLSVTCTVSGVSLPDYGVSWIRQ | |
| 41BB; CD3zeta) | PPRKGLEWLGVIWGSETTYYNSALKSRLTII | |
| | KDNSKSQVFLKMNSLQTDDTAIYYCAKHY | |
| | YYGGSYAMDYWGQGTSVTVSSGVQVETIS | |
| | PGDGRTFPKRGQTCVVHYTGMLEDGKKV | |
| | DSSRDRNKPFKFMLGKQEVIRGWEEGVAQ | |
| | MSVGQRAKLTISPDYAYGATGHPGIIPPHAT | |
| | LVFDVELLKPETTTPAPRPPTPAPTIASQPL | |
| | SLRPEACRPAAGGAVHTRGLDFACDIYIWA | |
| | PLAGTCGVLLLSLVITLYCKRGRKKLLYIFK | |
| | QPFMRPVQTTQEEDGCSCRFPEEEEGGCELR | |
| | VKFSRSADAPAYKQGQNQLYNELNLGRRE | |
| | EYDVLDKRRGRDPEMGGKPRRKNPQEGLY | |
| | NELQKDKMAEAYSEIGMKGERRRGKGHD | |
| | GLYQGLSTATKDTYDALHMQALPPR | |
| OT-CD19CAR- | MALPVTALLLPLALLLHAARPDIQMTQTTS | 37 |
| 003 (CD8a | SLSASLGDRVTISCRASQDISKYLNWYQQK | |
| leader; CD19 | PDGTVKLLIYHTSRLHSGVPSRFSGSGSGTD | |
| scFV; ecDHFR | YSLTISNLEQEDIATYFCQQGNTLPYTFGGG | |
| (R12Y, Y100I); | TKLEITGGGGSGGGGSGGGGSEVKLQESGP | |
| CD8a hinge + TM; | GLVAPSQSLSVTCTVSGVSLPDYGVSWIRQ | |
| 41BB; CD3zeta) | PPRKGLEWLGVIWGSETTYYNSALKSRLTII | |
| | KDNSKSQVFLKMNSLQTDDTAIYYCAKHY | |
| | YYGGSYAMDYWGQGTSVTVSSISLIAALA | |
| | VDYVIGMENAMPWNLPADLAWFKRNTLN | |
| | KPVIMGRHTWESIGRPLPGRKNIILSSQPGT | |
| | DDRVTWVKSVDEAIAACGDVPEIMVIGGG | |
| | RVIEQFLPKAQKLYLTHIDAEVEGDTHFPD | |
| | YEPDDWESVFSEFHDADAQNSHSYCFEILE | |
| | RRTTTPAPRPPTPAPTIASQPLSLRPEACRPA | |
| | AGGAVHTRGLDFACDIYIWAPLAGTCGVLLL | |
| | SLVITLYCKRGRKKLLYIFKQPFMRPVQTTQ | |
| | EEDGCSCRFPEEEEGGCELRVKFSRSADAP | |
| | AYKQGQNQLYNELNLGRREEYDVLDKRR | |
| | GRDPEMGGKPRRKNPQEGLYNELQKDKM | |
| | AEAYSEIGMKGERRRGKGHDGLYQGLSTA | |
| | TKDTYDALHMQALPPR | |
| OT-CD19 CAR- | MALPVTALLLPLALLLHAARPDIQMTQTTS | 38 |
| 004 (CD8a | SLSASLGDRVTISCRASQDISKYLNWYQQK | |
| leader; CD19 | PDGTVKLLIYHTSRLHSGVPSRFSGSGSGTD | |
| scFV; CD8a | YSLTISNLEQEDIATYFCQQGNTLPYTFGGG | |
| hingeFKBP | TKLEITGGGGSGGGGSGGGGSEVKLQESGP | |
| (F36V, L106P) | GLVAPSQSLSVTCTVSGVSLPDYGVSWIRQ | |
| CD8a TM- CD8 | PPRKGLEWLGVIWGSETTYYNSALKSRLTII | |
| hingeseq | KDNSKSQVFLKMNSLQTDDTAIYYCAKHY | |
| following TM; | YYGGSYAMDYWGQGTSVTVSSTTTPAPRP | |
| 41BB; CD3zeta) | PTPAPTIASQPLSLRPEACRPAAGGAVHTRG | |
| | LDFACDGVQVETISPGDGRTFPKRGQTCVV | |
| | HYTGMLEDGKKVDSSRDRNKPFKFMLGK | |
| | QEVIRGWEEGVAQMSVGQRAKLTISPDYA | |
| | YGATGHPGIIPPHATLVFDVELLKPEIYIWAP | |
| | LAGTCGVLLLSLVITLYCKRGRKKLLYIFKQP | |
| | FMRPVQTTQEEDGCSCRFPEEEEGGCELRV | |
| | KFSRSADAPAYKQGQNQLYNELNLGRREE | |
| | YDVLDKRRGRDPEMGGKPRRKNPQEGLY | |
| | NELQKDKMAEAYSEIGMKGERRRGKGHD | |
| | GLYQGLSTATKDTYDALHMQALPPR | |
| OT-CD19 CAR- | MALPVTALLLPLALLLHAARPDIQMTQTTS | 39 |
| 005 (CD8a | SLSASLGDRVTISCRASQDISKYLNWYQQK | |
| leader; CD19 | PDGTVKLLIYHTSRLHSGVPSRFSGSGSGTD | |
| scFV; ecDHFR | YSLTISNLEQEDIATYFCQQGNTLPYTFGGG | |
| (R12Y; Y100I); | TKLEITGGGGSGGGGSGGGGSEVKLQESGP | |
| 41BB; CD3zeta) | GLVAPSQSLSVTCTVSGVSLPDYGVSWIRQ | |
| | PPRKGLEWLGVIWGSETTYYNSALKSRLTII | |
| | KDNSKSQVFLKMNSLQTDDTAIYYCAKHY | |
| | YYGGSYAMDYWGQGTSVTVSSTTTPAPRP | |
| | PTPAPTIASQPLSLRPEACRPAAGGAVHTRG | |
| | LDFACDISLIAALAVDYVIGMENAMPWNLP | |
| | ADLAWFKRNTLNKPVIMGRHTWESIGRPL | |
| | PGRKNIILSSQPGTDDRVTWVKSVDEAIAA | |
| | CGDVPEIMVIGGGRVIEQFLPKAQKLYLTHI | |
| | DAEVEGDTHFPDYEPDDWESVFSEFHDAD | |
| | AQNSHSYCFEILERRIYIWAPLAGTCGVLLLS | |
| | LVITLYCKRGRKKLLYIFKQPFMRPVQTTQE | |
| | EDGCSCRFPEEEEGGCELRVKFSRSADAPA | |
| | YKQGQNQLYNELNLGRREEYDVLDKRRG | |
| | RDPEMGGKPRRKNPQEGLYNELQKDKMA | |
| | EAYSEIGMKGERRRGKGHDGLYQGLSTAT | |
| | KDTYDALHMQALPPR | |
| OT-CD19C | MALPVTALLLPLALLLHAARPDIQMTQTTS | 40 |
| CAR-006 (CD8a | SLSASLGDRVTISCRASQDISKYLNWYQQK | |
| leader; CD19 | PDGTVKLLIYHTSRLHSGVPSRFSGSGSGTD | |
| scFV; CD8a | YSLTISNLEQEDIATYFCQQGNTLPYTFGGG | |
| hinge + TM; | TKLEITGGGGSGGGGSGGGGSEVKLQESGP | |
| 41BB; CD3zeta; | GLVAPSQSLSVTCTVSGVSLPDYGVSWIRQ | |
| linker1 (GGSGG) | PPRKGLEWLGVIWGSETTYYNSALKSRLTII | |
| ecDHFR (R12H; | KDNSKSQVFLKMNSLQTDDTAIYYCAKHY | |
| E129K)) | YYGGSYAMDYWGQGTSVTVSSTTTPAPRP | |
| | PTPAPTIASQPLSLRPEACRPAAGGAVHTRG | |
| | LDFACDIYIWAPLAGTCGVLLLSLVITLYCKR | |
| | GRKKLLYIFKQPFMRPVQTTQEEDGCSCRF | |
| | PEEEEGGCELRVKFSRSADAPAYKQGQNQ | |
| | LYNELNLGRREEYDVLDKRRGRDPEMGGK | |
| | PRRKNPQEGLYNELQKDKMAEAYSEIGMK | |
| | GERRRGKGHDGLYQGLSTATKDTYDALH | |
| | MQALPPRGGSGGISLIAALAVDHVIGMENA | |
| | MPWNLPADLAWFKRNTLNKPVIMGRHTW | |
| | ESIGRPLPGRKNIILSSQPGTDDRVTWVKSV | |
| | DEAIAACGDVPEIMVIGGGRVYEQFLPKAQ | |
| | KLYLTHIDAEVEGDTHFPDYKPDDWESVFs | |
| | EFHDADAQNSHSYCFEILERR | |
| OT-CD19C-007 | MALPVTALLLPLALLLHAARPDIQMTQTTS | 41 |
| (CD8a leader; | SLSASLGDRVTISCRASQDISKYLNWYQQK | |
| CD19scFV; | PDGTVKLLIYHTSRLHSGVPSRFSGSGSGTD | |
| CD8a hinge + TM; | YSLTISNLEQEDIATYFCQQGNTLPYTFGGG | |
| 41BB; CD3zeta; | TKLEITGGGGSGGGGSGGGGSEVKLQESGP | |
| linker1 | GLVAPSQSLSVTCTVSGVSLPDYGVSWIRQ | |
| (GGSGG); FKBP | PPRKGLEWLGVIWGSETTYYNSALKSRLTII | |
| (E31G, F36V, | KDNSKSQVFLKMNSLQTDDTAIYYCAKHY | |
| R71G, K105E)) | YYGGSYAMDYWGQGTSVTVSSTTTPAPRP | |
| | PTPAPTIASQPLSLRPEACRPAAGGAVHTRG | |
| | LDFACDIYIWAPLAGTCGVLLLSLVITLYCKR | |
| | GRKKLLYIFKQPFMRPVQTTQEEDGCSCRF | |
| | PEEEEGGCELRVKFSRSADAPAYKQGQNQ | |
| | LYNELNLGRREEYDVLDKRRGRDPEMGGK | |
| | PRRKNPQEGLYNELQKDKMAEAYSEIGMK | |
| | GERRRGKGHDGLYQGLSTATKDTYDALH | |
| | MQALPPRGGSGGGVQVETISPGDGRTFPKR | |
| | GQTCVVHYTGMLGDGKKVDSSRDRNKPF | |
| | KFMLGKQEVIRGWEEGVAQMSVGQGAKL | |
| | TISPDYAYGATGHPGIIPPHATLVFDVELLE | |
| | LE | |

In one embodiment, the payload of the invention may be a TCR specific for the NY-ESO-1 and LAGE-1 cancer testis antigens (NY-ESOc259-T) that induces robust effector and memory T cells' expansion without inducing T cell exhaustion (See Melchiori et al. (2015) Molecular Therapy. 23, Sup1, p S204-S205. (the contents of which are incorporated herein by reference in their entirety).

In some embodiments, pharmaceutical compositions, biocircuits, biocircuit components, effector modules including their SREs or payloads of the present invention may be used in the modulation or alteration or exploitation of the immune system to target one or more self-reactive immune components such as auto antibodies and self-reactive immune cells to attenuate autoimmune diseases. In some embodiments, the SREs of the present invention may be utilized in regulating or tuning the Chimeric Auto Antibody Receptor (CAAR) based T cell therapy in order to optimize its utility in the treatment of autoimmune diseases (Ellebrecht C. T. et al., Science. 2016. Jul. 8; 353(6295):179-84; the contents of which are incorporated herein by reference in their entirety). In some embodiments, biocircuits, their components, SREs or effector modules are designed to modulate Tregs to attenuate autoimmune disorders. In such a case, IL-2 may be regulated using a singly tuned module or one having multiple tuned features as described herein.

In some embodiments, biocircuits, their components, SREs or effector modules may be utilized in immunotherapy-based treatments to attenuate or mitigate Graft vs. Host disease (GVHD). GVHD refers to a condition following stem cell or bone marrow transplant where in the allogeneic donor immune cells react against host tissue. In some embodiments, biocircuits, their components, SREs or effector modules are designed to modulate Tregs for the treatment of GVHD. In one embodiment, biocircuits containing an effector module encoding TNF-alpha may be used to modulate Tregs to minimize GVHD (Pierini, A. et al., Blood. 2016. Aug. 11; 128(6):866-71; the contents of which are incorporated herein by reference in their entirety).

In some embodiments, biocircuits, their components, SREs or effector modules are designed to be significantly less immunogenic than other biocircuits or switches in the art.

As used herein, “significantly less immunogenic” refers to a detectable decrease in immunogenicity. In another embodiment, the term refers to a fold decrease in immunogenicity. In another embodiment, the term refers to a decrease such that an effective amount of the biocircuits, their components, SREs or effector modules which can be administered without triggering a detectable immune response. In another embodiment, the term refers to a decrease such that the biocircuits, their components, SREs or effector modules can be repeatedly administered without eliciting an immune response. In another embodiment, the decrease is such that the biocircuits, their components, SREs or effector modules can be repeatedly administered without eliciting an immune response.

In another embodiment, the biocircuits, their components, SREs or effector modules is 2-fold less immunogenic than its unmodified counterpart or reference compound. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

Methods of determining immunogenicity are well known in the art, and include, e.g. measuring secretion of cytokines (e.g. IL-12, IFNalpha, TNF-alpha, RANTES, MIP-1alpha or beta, IL-6, IFN-beta, or IL-8), measuring expression of DC activation markers (e.g. CD83, HLA-DR, CD80 and CD86), or measuring ability to act as an adjuvant for an adaptive immune response.

In one embodiment, the chimeric antigen receptor (CAR) of the present invention may be a conditionally active CAR. A wild type protein or domain thereof, such as those described herein may be used to generate a conditionally active biologic protein which are reversibly or irreversibly inactivated at the wild type normal physiological conditions as well as to such conditionally active biologic proteins and domains and uses of such conditional active biologic proteins and domains are provided. Such methods and conditionally active proteins are taught in, for example, International Publication No. WO2016033331, the contents of which are incorporated herein by reference in their entirety. As a non-limiting example, the CAR comprises at least one antigen specific targeting region evolved from a wild type protein or a domain thereof and one or more of a decrease in activity in the assay at the normal physiological condition compared to the antigen specific targeting region of the wild-type protein or a domain thereof, and an increase in activity in the assay under the aberrant condition compared to the antigen specific targeting region of the wild-type protein or a domain thereof.

### Diseases and Toxins

Various infectious diseases may be treated with pharmaceutical compositions, biocircuits, biocircuit components, effector modules including their SREs or payloads of the present invention. As used herein, the term “infectious disease” refers to any disorders caused by organisms such as bacteria, viruses, fungi or parasites. As a non-limiting example, the infectious disease may be Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis—(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium difficile Infection, Clostridium perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. Coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin—B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes—genital, Herpes labialis, Herpes—neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis—Swimming Pool conjunctivitis-Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease—lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis—Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers—Buruli ulcers, Mycotic vulvovaginitis, Myositis, Nanukayami fever, Necrotizing fasciitis, Necrotizing fasciitis—Type 1, Necrotizing fasciitis—Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra (Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans, Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium tetani, Bordetella pertussis, Bordetella paratussis, Mycobacterium, Francisella tularensis, Toxoplasma gondii, Candida (C. albicans, C. glabrata, C. parapsilosis, C. tropicalis, C. krusei and C. lusitaniae) and/or any other infectious diseases, disorders or syndromes.

Various toxins may be treated with pharmaceutical compositions, biocircuits, biocircuit components, effector modules including their SREs or payloads of the present invention. Non-limited examples of toxins include Ricin, Bacillus anthracis, Shiga toxin and Shiga-like toxin, Botulinum toxins.

Various tropical diseases may be treated with pharmaceutical compositions, biocircuits, biocircuit components, effector modules including their SREs or payloads of the present invention. Non-limited examples of tropical diseases include Chikungunya fever, Dengue fever, Chagas disease, Rabies, Malaria, Ebola virus, Marburg virus, West Nile Virus, Yellow Fever, Japanese encephalitis virus, St. Louis encephalitis virus.

Various foodborne illnesses and gastroenteritis may be treated with pharmaceutical compositions, biocircuits, biocircuit components, effector modules including their SREs or payloads of the present invention. Non-limited examples of foodborne illnesses and gastroenteritis include Rotavirus, Norwalk virus (Norovirus), Campylobacter jejuni, Clostridium difficile, Entamoeba histolytica, Helicobacter pylori, Enterotoxin B of Staphylococcus aureus, Hepatitis A virus (HAV), Hepatitis E, Listeria monocytogenes, Salmonella, Clostridium perfringens, and Salmonella.

Various infectious agents may be treated with pharmaceutical compositions, biocircuits, biocircuit components, effector modules including their SREs or payloads of the present invention. Non-limited examples of infectious agents include adenoviruses, Anaplasma phagocytophilium, Ascaris lumbricoides, Bacillus anthracis, Bacillus cereus, Bacteroides sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis., Ehrlichia equi., Ehrlichia sp., Entamoeba histolytica, Enterobacter sp., Enterococcus faecalis, Enterovirus 71, Epstein-Barr virus (EBV), Erysipelothrix rhusiopathiae, Escherichia coli, Flavivirus, Fusobacterium necrophorum, Gardnerella vaginalis, Group B Streptococcus, Haemophilus aegyptius, Haemophilus ducreyi, Haemophilus influenzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunninham virus, juninvirus, Kaposi's Sarcoma-associated Herpes Virus (KSHV), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MERS coronavirus (MERS-CoV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types I, II, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella typhi, Sarcoptes scabiei, SARS-associated coronavirus (SARS-CoV), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptococcus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urealyticum, Varicella-Zoster virus, variola virus, Vibrio cholerae, West Nile virus, yellow fever virus, Yersinia enterocolitica, Yersinia pestis, and Zika virus.

Various rare diseases may be treated with pharmaceutical compositions, biocircuits, biocircuit components, effector modules including their SREs or payloads of the present invention. As used herein, the term “rare disease” refers to any disease that affects a small percentage of the population. As a non-limiting example, the rare disease may be Acrocephalosyndactylia, Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia, Arnold-Chiari Malformation, Arthritis, Juvenile Rheumatoid, Asperger Syndrome, Bardet-Biedl Syndrome, Barrett Esophagus, Beckwith-Wiedemann Syndrome, Behcet Syndrome, Bloom Syndrome, Bowen's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome, Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felty's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VII, Goldenhar Syndrome, Guillain-Barre Syndrome, Hallermann's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Horner Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau-Kleffner Syndrome, Langer-Giedion Syndrome, Leigh Disease, Lesch-Nyhan Syndrome, Leukodystrophy, Globoid Cell, Li-Fraumeni Syndrome, Long QT Syndrome, Machado-Joseph Disease, Mallory-Weiss Syndrome, Marek Disease, Marfan Syndrome, Meckel Diverticulum, Meige Syndrome, Melkersson-Rosenthal Syndrome, Meniere Disease, Mikulicz' Disease, Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis III, Mucopolysaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Osteoarthritis, Essential, Paget Disease Extramammary, Paget's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders, Peutz-Jeghers Syndrome, Pick Disease of the Brain, Pierre Robin Syndrome, Pigmentation Disorders, Pityriasis Lichenoides, Polycystic Ovary Syndrome, Polyendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewing's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Sweating, Gustatory, Takayasu Arteritis, Tangier Disease, Tay-Sachs Disease, Thromboangiitis Obliterans, Thyroiditis, Autoimmune, Tietze's Syndrome, Togaviridae Infections, Tolosa-Hunt Syndrome, Tourette Syndrome, Uveomeningoencephalitic Syndrome, Waardenburg's Syndrome, Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

Various autoimmune diseases and autoimmune-related diseases may be treated with pharmaceutical compositions, biocircuits, biocircuit components, effector modules including their SREs or payloads of the present invention. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Various kidney diseases may be treated with pharmaceutical compositions, biocircuits, biocircuit components, effector modules including their SREs or payloads of the present invention. As a non-limiting example, the kidney disease Abderhalden-Kaufmann-Lignac syndrome (Nephropathic Cystinosis), Abdominal Compartment Syndrome, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Bartter Syndrome, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Cherry Concentrate and Acute Kidney Injury, Cholesterol Emboli, Churg-Strauss syndrome, Chyluria, Colistin Nephrotoxicity, Collagenofibrotic Glomerulopathy, Collapsing Glomerulopathy, Collapsing Glomerulopathy Related to CMV, Congenital Nephrotic Syndrome, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryoglobuinemia, Crystalglobulin-Induced Nephropathy, Crystal-Induced Acute Kidney injury, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), Dialysis Disequilibrium Syndrome, Diabetes and Diabetic Kidney Disease, Diabetes Insipidus, Dietary Supplements and Renal Failure, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Hair Dye Ingestion and Acute Kidney Injury, Hantavirus Infection Podocytopathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatorenal Syndrome, Herbal Supplements and Kidney Disease, High Blood Pressure and Kidney Disease, HIV-Associated Nephropathy (HIVAN), Horseshoe Kidney (Renal Fusion), Hunner's Ulcer, Hyperaldosteronism, Hypercalcemia, Hyperkalemia, Hypermagnesemia, Hypernatremia, Hyperoxaluria, Hyperphosphatemia, Hypocalcemia, Hypokalemia, Hypokalemia-induced renal dysfunction, Hypokalemic Periodic Paralysis, Hypomagnesemia, Hyponatremia, Hypophosphatemia, IgA Nephropathy, IgG4 Nephropathy, Interstitial Cystitis, Painful Bladder Syndrome (Questionnaire), Interstitial Nephritis, Ivemark's syndrome, Ketamine-Associated Bladder Dysfunction, Kidney Stones, Nephrolithiasis, Kombucha Tea Toxicity, Lead Nephropathy and Lead-Related Nephrotoxicity, Leptospirosis Renal Disease, Light Chain Deposition Disease, Monoclonal Immunoglobulin Deposition Disease, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lyme Disease-Associated Glomerulonephritis, Malarial Nephropathy, Malignancy-Associated Renal Disease, Malignant Hypertension, Malakoplakia, Meatal Stenosis, Medullary Cystic Kidney Disease, Medullary Sponge Kidney, Megaureter, Melamine Toxicity and the Kidney, Membranoproliferative Glomerulonephritis, Membranous Nephropathy, MesoAmerican Nephropathy, Metabolic Acidosis, Metabolic Alkalosis, Methotrexate-related Renal Failure, Microscopic Polyangiitis, Milk-alkalai syndrome, Minimal Change Disease, MDMA (Molly; Ecstacy; 3,4-Methylenedioxymethamphetamine) and Kidney Failure, Multicystic dysplastic kidney, Multiple Myeloma, Myeloproliferative Neoplasms and Glomerulopathy, Nail-patella Syndrome, Nephrocalcinosis, Nephrogenic Systemic Fibrosis, Nephroptosis (Floating Kidney, Renal Ptosis), Nephrotic Syndrome, Neurogenic Bladder, Nodular Glomerulosclerosis, Non-Gonococcal Urethritis, Nutcracker syndrome, Orofaciodigital Syndrome, Orotic Aciduria, Orthostatic Hypotension, Orthostatic Proteinuria, Osmotic Diuresis, Ovarian Hyperstimulation Syndrome, Page Kidney, Papillary Necrosis, Papillorenal Syndrome (Renal-Coloboma Syndrome, Isolated Renal Hypoplasia), Parvovirus B19 and the Kidney, The Peritoneal-Renal Syndrome, Posterior Urethral Valve, Post-infectious Glomerulonephritis, Post-streptococcal Glomerulonephritis, Polyarteritis Nodosa, Polycystic Kidney Disease, Posterior Urethral Valves, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjögren's Syndrome and Renal Disease, Synthetic Cannabinoid Use and Acute Kidney Injury, Kidney Disease Following Hematopoietic Cell Transplantation, Kidney Disease Related to Stem Cell Transplantation, Thin Basement Membrane Disease, Benign Familial Hematuria, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Vesicointestinal Fistula, Vesicoureteral Reflux, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, and Wunderlich syndrome.

Various cardiovascular diseases may be treated with pharmaceutical compositions, biocircuits, biocircuit components, effector modules including their SREs or payloads of the present invention. As a non-limiting example, the cardiovascular disease may be Ischemic heart disease also known as coronary artery disease, Cerebrovascular disease (Stroke), Peripheral vascular disease, Heart failure, Rheumatic heart disease, and Congenital heart disease.

Various antibody deficiencies may be treated with pharmaceutical compositions, biocircuits, biocircuit components, effector modules including their SREs or payloads of the present invention. As a non-limiting example, the antibody deficiencies may be X-Linked Agammaglobulinemia (XLA), Autosomal Recessive Agammaglobulinemia (ARA), Common Variable Immune Deficiency (CVID), IgG (IgG1, IgG2, IgG3 and IgG4) Subclass Deficiency, Selective IgA Deficiency, Specific Antibody Deficiency (SAD), Transient Hypogammaglobulinemia of Infancy, Antibody Deficiency with Normal or Elevated Immunoglobulins, Selective IgM Deficiency, Immunodeficiency with Thymoma (Good's Syndrome), Transcobalamin II Deficiency, Warts, Hypogammaglobulinemia, Infection, Myelokathexis (WHIM) Syndrome, Drug-Induced Antibody Deficiency, Kappa Chain Deficiency, Heavy Chain Deficiencies, Post-Meiotic Segregation (PMS2) Disorder, and Unspecified Hypogammaglobulinemia.

Various ocular diseases may be treated with pharmaceutical compositions, biocircuits, biocircuit components, effector modules including their SREs or payloads of the present invention. As a non-limiting example, the ocular disease may be thyroid eye disease (TED), Graves' disease (GD) and orbitopathy, Retina Degeneration, Cataract, optic atrophy, macular degeneration, Leber congenital amaurosis, retinal degeneration, cone-rod dystrophy, Usher syndrome, leopard syndrome, photophobia, and photoaversion.

Various neurological diseases may be treated with pharmaceutical compositions, biocircuits, biocircuit components, effector modules including their SREs or payloads of the present invention. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

Various psychological disorders may be treated with pharmaceutical compositions, biocircuits, biocircuit components, effector modules including their SREs or payloads of the present invention. As a non-limiting example, the psychological disorders may be Aboulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder), Amphetamine Addiction, Anorexia Nervosa, Anterograde amnesia, Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADHD), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder, Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder), Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermotillomania, Desynchronosis, Developmental coordination disorder, Diogenes Syndrome, Disorder of written expression, Dispareunia, Dissocial Personality Disorder, Dissociative Amnesia, Dissociative Fugue, Dissociative Identity Disorder (formerly known as Multiple Personality Disorder), Down syndrome, Dyslexia, Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania, Exhibitionistic Disorder, Expressive language disorder, Factitious Disorder, Female Sexual Disorders, Fetishistic Disorder, Folie à deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haltlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder, Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease, Partner relational problem, Passive-aggressive personality disorder, Pathological gambling, Pedophilic Disorder, Perfectionism, Persecutory delusion, Persistent Depressive Disorder (also known as Dysthymia), Personality change due to a general medical condition, Personality disorder, Pervasive developmental disorder (PDD), Phencyclidine related disorder, Phobic disorder, Phonological disorder, Physical abuse, Pica, Polysubstance related disorder, Postpartum Depression, Post-traumatic embitterment disorder (PTED), Post-Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder, Schizotypal Personality Disorder, Seasonal Affective Disorder, Sedative, Hypnotic, or Anxiolytic Addiction, Selective Mutism, Self-defeating personality disorder, Separation Anxiety Disorder, Sexual Disorders Female, Sexual Disorders Male, Sexual Addiction, Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias, Stendhal syndrome, Stereotypic movement disorder, Stimulant Addiction, Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder.

Various lung diseases may be treated with pharmaceutical compositions, biocircuits, biocircuit components, effector modules including their SREs or payloads of the present invention. As a non-limiting example, the lung diseases may be Asbestosis, Asthma, Bronchiectasis, Bronchitis, Chronic Cough, Chronic Obstructive Pulmonary Disease (COPD), Croup, Cystic Fibrosis, Hantavirus, Idiopathic Pulmonary Fibrosis, Pertussis, Pleurisy, Pneumonia, Pulmonary Embolism, Pulmonary Hypertension, Sarcoidosis, Sleep Apnea, Spirometry, Sudden Infant Death Syndrome (SIDS), Tuberculosis, Alagille Syndrome, Autoimmune Hepatitis, Biliary Atresia, Cirrhosis, ERCP (Endoscopic Retrograde Cholangiopancreatography), and Hemochromatosis. Nonalcoholic Steatohepatitis, Porphyria, Primary Biliary Cirrhosis, Primary Sclerosing Cholangitis.

Various bone diseases may be treated with pharmaceutical compositions, biocircuits, biocircuit components, effector modules including their SREs or payloads of the present invention. As a non-limiting example, the bone diseases may be osteoporosis, neurofibromatosis, osteogenesis imperfecta (OI), rickets, osteosarcoma, achondroplasia, fracture, osteomyelitis, Ewing tumor of bone, osteomalacia, hip dysplasia, Paget disease of bone, marble bone disease, osteochondroma, bone cancer, bone disease, osteochondrosis, osteoma, fibrous dysplasia, cleidocranial dysostosis, osteoclastoma, bone cyst, metabolic bone disease, melorheostosis, callus, Caffey syndrome, and mandibulofacial dysostosis.

Various blood diseases may be treated with pharmaceutical compositions, biocircuits, biocircuit components, effector modules including their SREs or payloads of the present invention. As a non-limiting example, the blood diseases may be Anemia and CKD (for health care professionals), Aplastic Anemia and Myelodysplastic Syndromes, Deep Vein Thrombosis, Hemochromatosis, Hemophilia, Henoch-Schonlein Purpura, Idiopathic Thrombocytopenic Purpura, Iron-Deficiency Anemia, Pernicious Anemia, Pulmonary Embolism, Sickle Cell Anemia, Sickle Cell Trait and Other Hemoglobinopathies, Thalassemia, Thrombotic Thrombocytopenic Purpura, and Von Willebrand Disease.

### Central Nervous System (CNS)

In some embodiments, pharmaceutical compositions, biocircuits, biocircuit components, effector modules including their SREs or payloads of the present invention may be used in the modulation or alteration or exploitation of proteins in the central nervous system including cerebrospinal (CSF) proteins.

In some examples, pharmaceutical compositions, biocircuits, biocircuit components, effector modules including their SREs or payloads of the present invention may be used to provide tunable ERT (enzyme replacement therapy) products to the central nervous system. Many lysosomal storage diseases (LSD) involve the CNS symptoms, such as mental retardation, seizures, profound neurodegeneration, behavioral abnormalities, and psycho-motor defects. ERT for LSDs is one of the true success stories in modern molecular medicine. The successful application of ERT relies on controlled lysosomal proteins (e.g., enzymes) and delivery to CNS cells. Compositions of the present invention may be used for ERT products for Mucopolysaccharidosis type II (Hunter Syndrome, iduronate sulfatase deficiency), Mucopolysaccharidosis type VI (Maroteaux-Lamy Syndrome, arylsulfatase B deficiency), Mucopolysaccharidosis type III (Sanfilippo A), Mucopolysaccharidosis type IV (MPS IV), Pompe disease (acid maltase deficiency), Niemann-Pick B (NP-B) disease, metachromatic leukodystrophy (MLD, Arylsufatase A deficiency), Krabbe disease, Wolman disease, and Sly syndrome.

In some examples, pharmaceutical compositions, biocircuits, biocircuit components, effector modules including their SREs or payloads of the present invention may be used to locally produce monoclonal antibodies against protein aggregates in the CNS and CSF. Such antibodies may be used to treat degenerative diseases like Alzheimer's disease (AD), Huntington's Disease (HD) and Parkinson's disease (PD).

In other examples, pharmaceutical compositions, biocircuits, biocircuit components, effector modules including their SREs or payloads of the present invention may be used to regulate neurotrophic factors in the central nervous system.

### Gene Editing

The CRISPR-Cas9 system is a novel genome editing system which has been rapidly developed and implemented in a multitude of model organisms and cell types, and supplants other genome editing technologies, such as TALENs and ZFNs. CRISPRs are sequence motifs are present in bacterial and archaeal genomes, and are composed of short (about 24-48 nucleotide) direct repeats separated by similarly sized, unique spacers (Grissa et al. BMC Bioinformatics 8, 172 (2007)). They are generally flanked by a set of CRISPR-associated (Cas) protein-coding genes that are required for CRISPR maintenance and function (Barrangou et al., Science 315, 1709 (2007), Brouns et al., Science 321, 960 (2008), Haft et al. PLoS Comput Biol 1, e60 (2005)). CRISPR-Cas systems provide adaptive immunity against invasive genetic elements (e.g., viruses, phages and plasmids) (Horvath and Barrangou, Science, 2010, 327: 167-170; Bhaya et al., Annu. Rev. Genet., 2011, 45: 273-297; and Brrangou R, RNA, 2013, 4: 267-278). Three different types of CRISPR-Cas systems have been classified in bacteria and the type II CRISPR-Cas system is most studied. In the bacterial Type II CRISPR-Cas system, small CRISPR RNAs (crRNAs) processed from the pre-repeat-spacer transcript (pre-crRNA) in the presence of a trans-activating RNA (tracrRNA)/Cas9 can form a duplex with the tracrRNA/Cas9 complex. The mature complex is recruited to a target double strand DNA sequence that is complementary to the spacer sequence in the tracrRNA: crRNA duplex to cleave the target DNA by Cas9 endonuclease (Garneau et al., Nature, 2010, 468: 67-71; Jinek et al., Science, 2012, 337: 816-821; Gasiunas et al., Proc. Natl Acad. Sci. USA., 109: E2579-2586; and Haurwitz et al., Science, 2010, 329: 1355-1358). Target recognition and cleavage by the crRNA: tracrRNA/Cas9 complex in the type II CRISPR-CAS system not only requires a sequence in the tracrRNA: crRNA duplex that is a complementary to the target sequence (also called “protospacer” sequence) but also requires a protospacer adjacent motif (PAM) sequence located 3′end of the protospacer sequence of a target polynucleotide. The PAM motif can vary between different CRISPR-Cas systems.

CRISPR-Cas9 systems have been developed and modified for use in genetic editing and prove to be a high effective and specific technology for editing a nucleic acid sequence even in eukaryotic cells. Many researchers disclosed various modifications to the bacterial CRISPR-Cas systems and demonstrated that CRISPR-Cas systems can be used to manipulate a nucleic acid in a cell, such as in a mammalian cell and in a plant cell. Representative references include U.S. Pat. Nos. 8,993,233; 8,999,641; 8,945,839; 8,932,814; 8,906,616; 8,895,308; 8,889,418; 8,889,356; 8,871,445; 8,865,406; 8,771,945; and 8,697,359; US patent publication NOs.: 20150031134; 20150203872; 20150218253; 20150176013; 20150191744; 20150071889; 20150067922; and 20150167000; each of which is incorporated herein by reference in their entirety.

However, controlling the effects and activity of the CRISPR-Cas system (e.g., guide RNA and nuclease) has been challenging and often can be problematic.

The biocircuits of the present invention and/or any of their components may be utilized in regulating or tuning the CRISPR/Cas9 system in order to optimize its utility.

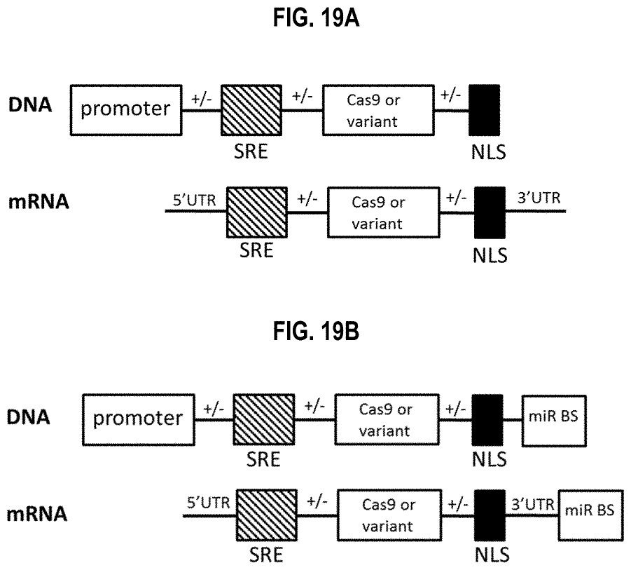

Examples for tuning the system are shown in FIG. 19A and FIG. 19B.

In some embodiments, the payloads of the effector modules of the invention may include alternative isoforms or orthologs of the Cas9 enzyme.

The most commonly used Cas9 is derived from Streptococcus pyogenes and the RuvC domain can be inactivated by a D10A mutation and the HNH domain can be inactivated by an H840A mutation.

In addition to Cas9 derived from S. pyogenes, other RNA guided endonucleases (RGEN) may also be used for programmable genome editing. Cas9 sequences have been identified in more than 600 bacterial strains. Though Cas9 family shows high diversity of amino acid sequences and protein sizes, All Cas9 proteins share a common architecture with a central HNH nuclease domain and a split RuvC/RHase H domain. Examples of Cas9 orthologs from other bacterial strains including but not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum_SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In some embodiments, the payload of the present invention may be a split Cas-9 (Zetsche B et al. A split-Cas9 architecture for inducible genome editing and transcription modulation. Nat Biotechnol. 2015 February; 33(2):139-42; the contents of which are incorporated by reference in their entirety).

In addition to Cas9 orthologs, other Cas9 variants such as fusion proteins of inactive dCas9 and effector domains with different functions may serve as a platform for genetic modulation. Any of the foregoing enzymes may be useful in the present invention.

CRISPR/Cas9 based biocircuits may be generated by any of the methods taught in International Publication No.: WO2016106244 and Gao Y et al. Complex transcriptional modulation with orthogonal and inducible dCas9 regulators. Nat Methods. 2016 December; 13(12):1043-1049; the contents of each of which are incorporated herein by reference in their entirety).

The CRISPR/Cas9 system may also be utilized to modulate gene expression, which may be combined with its gene editing utility. In some embodiments, the payloads of the effector modules of the invention may include CRISPR associated transcriptional activators e.g VP64-p65-Rta (VPR); or repressors e.g Kruppel-associated box (KRAB) associated with the CRISPR/Cas9 system.

### Stem Cell Applications

The biocircuits of the present invention and/or any of their components may be utilized in the regulated reprogramming of cells, stem cell engraftment or other application where controlled or tunable expression of such reprogramming factors are useful.

The biocircuits of the present invention may be used in reprogramming cells including stem cells or induced stem cells. Induction of induced pluripotent stem cells (iPSC) was first achieved by Takahashi and Yamanaka (Cell, 2006. 126(4):663-76; herein incorporated by reference in its entirety) using viral vectors to express KLF4, c-MYC, OCT4 and SOX2 otherwise collectively known as KMOS.

Excisable lentiviral and transposon vectors, repeated application of transient plasmid, episomal and adenovirus vectors have also been used to try to derive iPSC (Chang, C.-W., et al., Stem Cells, 2009. 27(5):1042-1049; Kaji, K., et al., Nature, 2009. 458(7239):771-5; Okita, K., et al., Science, 2008. 322(5903):949-53; Stadtfeld, M., et al., Science, 2008. 322(5903):945-9; Woltjen, K., et al., Nature, 2009; Yu, J., et al., Science, 2009:1172482; Fusaki, N., et al., Proc Jpn Acad Ser B Phys Biol Sci, 2009. 85(8):348-62; each of which is herein incorporated by reference in its entirety).

DNA-free methods to generate human iPSC has also been derived using serial protein transduction with recombinant proteins incorporating cell-penetrating peptide moieties (Kim, D., et al., Cell Stem Cell, 2009. 4(6): 472-476; Zhou, H., et al., Cell Stem Cell, 2009. 4(5):381-4; each of which is herein incorporated by reference in its entirety), and infectious transgene delivery using the Sendai virus (Fusaki, N., et al., Proc Jpn Acad Ser B Phys Biol Sci, 2009. 85(8): p. 348-62; herein incorporated by reference in its entirety).

The effector modules of the present invention may include a payload comprising any of the genes including, but not limited to, OCT such as OCT4, SOX such as SOX1, SOX2, SOX3, SOX15 and SOX18, NANOG, KLF such as KLF1, KLF2, KLF4 and KLF5, MYC such as c-MYC and n-MYC, REM2, TERT and LIN28 and variants thereof in support of reprogramming cells. Sequences of such reprogramming factors are taught in for example International Application PCT/US2013/074560, the contents of which are incorporated herein by reference in their entirety.

The effector modules of the present invention may include a payload comprising any of factors that contribute stem cell mobilization. In autologous stem cell therapy, sources of stem cells for transplantation may include the bone marrow, peripheral blood mononuclear cells and cord blood. Stem cells are stimulated out of these sources (e.g., the bone marrow) into the blood stream. So sufficient stem cells are available for collection for future reinfusion. One or a combination of cytokines strategies may be used to mobilize the stem cells including but not limited to G-CSF (filgrastim), GM-CSF, and chemotherapy preceding with cytokines (chemomobilization).

### Metabolic Peptides and Hormones

In some embodiments, the biocircuits of the present invention and/or any of their components may be used to regulate peptides, natural or synthetic. Naturally occurring peptides may include but are not limited to, peptide hormones, natriuretic peptides, food peptides, and derivatives and precursors. Peptide hormones may be selected from amylin (IAPP), anti-mullerian hormone (AMH), adiponectin (Acrp30), adrenocorticotropic hormone (ACTH), angiotensinogen (AGT), antidiuretic hormone (ADH), Aprosin, atriopeptin (ANP), brain natriuretic peptide (BNP), calcitonin (CT), cholecystokinin (CCK), corticotropin-releasing hormone (CRH), cortistatin (CORT), encephalin, endothelin, erythropoietin (EPO), follicle-stimulating hormone (FSH), galanin (GAL), gastric inhibitory polypeptide (GIP), gastrin (GRP), ghrelin, glucagon (GCG), Glucagon-like peptide-1 (GLP1), gonadotropin-releasing hormone (GnRH), growth hormone-releasing hormone (GHRH), guanylin (GN), hepcidin (HAMP), human chorionic gonadotropin (HPL), growth hormone (GH), inhibin, insulin (INS), somatomedin, leptin (LEP), lipotropin (LPH), luteinizing hormone (LH), melanocyte stimulating hormone (MSH), motilin (MLN), orexin, oxytocin (OXT), pancreatic polypeptide, parathyroid hormone (PTH), pituitary adenylate cyclase-activating peptide (PACAP), prolactin (PRL), prolactin releasing hormone (PRH), relaxin (RLN), renin, secretin (SCT), Thrombopoietin (TPO), thyroid-stimulating hormone (TSH), thyrotropin-releasing hormone (TRH), vasoactive intestinal peptide (VIP), and uroguanylin (UGN). Natriuretic peptides may be selected from Cardiodilatin related peptide (CDD) (also known as atrial natriuretic peptide (ANP)), BNP and C-type natriuretic peptide (CNP) and Urodilatin.

Other peptides may include host defense peptides (HDPs) (also called antimicrobial peptides, AMPs), and other naturally occurring peptides.

The biocircuits of the present invention and/or any of their components may also be utilized for pulsatile release of hormones or other peptide drugs.

### Enzyme Replacement Therapy (ERT)

Enzyme replacement therapy (ERT) is a medical treatment replacing an enzyme in a patient. ERT provides therapeutic interventions that address the underlying metabolic defect in many disorders caused by defective enzymes. Such disorders include, but are not limited to, lysosomal storage diseases (LSDs), congenital disorders of glycosylation, and metabolic disorders characterized by missing or reduced enzyme activity in the cytoplasm. Non-limiting examples of lysosomal storage diseases include: Activator Deficiency; Alpha-mannosidosis, Aspartylglucosaminuria, Cholesteryl ester storage disease, Chronic Hexosaminidase A Deficiency, Cystinosis, Danon disease, Gaucher disease, Fabry disease, Farber disease; Fucosidosis; Galactosialidosis, GMl gangliosidosis, I-Cell disease, Infantile Free Sialic Acid Storage Disease, Krabbe disease, Metachromatic Leukodystrophy, Pompe disease, Mucopolysaccharidosis I, Hurler syndrome, Hurler-Scheie syndrome, Scheie syndrome, Mucopolysaccharidosis II, Hunter syndrome, Mucopolysaccharidosis IV, Mucopolysaccharidosis VI, Lysosomal Acid lipase deficiency, Thrombocytopenia, Maroteaux-Lamy syndrome, Sly syndrome, Pycnodysostosis, Sandhoff disease, Schindler disease, Salla disease, Tay-Sachs, and Wolman disease.

In some embodiments, the biocircuits of the present invention and/or any of their components may also be utilized to regulate enzymatic activities during ERT. As a non-limiting example, payloads of the biocircuits of the present invention may be a functional lysosomal enzyme for ERT, such as a-D-mannosidase, N-aspartyl-β-glucosaminidase, acid lipase; hexosaminidase A, a-galactosidase A, β-galactosidase, lysosomal protease, ceramidase, fucosidase; β-glucosidase, N-acetylglucosamine-1-phosphotransferase, sulfatase, hyaluronidase, galactocerebrosidase; arylsulfatase A; N-acetylglucosamine-1-phosphotransferase; a-L-iduronidase; iduronate sulfatase; heparan sulfamidase; N-acetylglucosaminidase; acetyl-CoA:a-glucosaminide acetyltransferase; N-acetylglucosamine 6-sulfatase; N-acetylgalactosamine-6-sulfate sulfatase; N-acetylgalactosamine-4-sulfatase; β-glucuronidase; hyaluronidase; sialidase; sulfatase; sphingomyelinase; acid a-glucosidase; β-mannosidase; cathepsin K; β-hexosaminidase A; β-hexosaminidase B, a-N-acetylgalactosaminidase, sialin, and hexosaminidase.

### Coagulation

Coagulation defects often cause hemorrhage and/or thrombosis. The best-known coagulation factor disorders are the hemophilias. The three main forms are hemophilia A (factor VIII deficiency), hemophilia B (factor IX deficiency or “Christmas disease”) and hemophilia C (factor XI deficiency, mild bleeding tendency). Other disorders caused by defective coagulation factors also include, but are not limited to, Von Willebrand disease (caused by a defect in von Willebrand factor (vWF), Bernard-Soulier syndrome (caused by a defect or deficiency in GPIb, a receptor of vWF), thrombophlebitis (caused by mutations in Factor XII), Congenital afibrinogenemia, Familial renal amyloidosis (caused by mutations in Factor I), congenital proconvertin/factor VII deficiency, Thrombophilia (caused by Factor II deficiency), Congenital Factor X deficiency, Congenital Factor XIIIa/b deficiency, Prekallikrein/Fletcher Factor deficiency, Kininogen deficiency, Glomerulopathy with fibronectin deposits, Heparin cofactor II deficiency, Protein C deficiency, Protein S deficiency, Protein Z deficiency, Antithrombin III deficiency, Plasminogen deficiency, type I (ligneous conjunctivitis), Antiplasmin deficiency, Plasminogen activator inhibitor-1 deficiency, and Quebec platelet disorder.

Gene therapy for coagulation factor replacement is a medical treatment of disorders caused be coagulation deficiency. In accordance with the present invention, the biocircuits of the present invention and/or any of their components may also be utilized to regulate a coagulation factor used for gene therapy. In some examples, the coagulation factor may be selected from Factor I (fibrinogen), Factor II (prothrombin), Factor III (tissue factor), Factor IV, Factor V (proaccelerin), Factor VI, Factor VII (stable factor), Factor VIII (antihemophilic factor A), Factor IX (antihemophilic factor B), Factor X (Stuart-Prower factor), Factor XI (plasma thromboplastin antecedent), Factor XII (Hageman factor), Factor XIII (fibrin-stabilizing factor), von Willebrand factor, Prekallikrein (Fletcher factor), high-molecular-weight kininogen (HMWK) (Fitzgerald factor), fibronectin, antithrombin III, heparin cofactor II, protein C, protein S, protein Z, protein Z related protease inhibitor (ZPI), plasminogen, tissue plasminogen activator (tPA), urokiase, plasminogen, plasminogen activator inhibitor 1 (PAI1), and plasminogen activator inhibitor 2 (PAI2)

In one embodiment, the coagulate factor is Factor VIII for gene therapy of hemophilia, including wild type factor VIII, engineered Factor VIII, activated fVIII (fVIIIa), or the equivalent. Exemplary engineered Factor VIII may include those discussed by Roberts et al (J. Genet. Syndr. Gene Ther., 2011, 1: S1-006; the contents of which are incorporated herein by reference in their entirety).

In another embodiment, the coagulate factor may be Factor IX for gene therapy of hemophilia B. The factor IX may be a recombinant factor IX as disclosed in U.S. Pat. Nos. 7,575,897; 7,700,734; 7,888,067; and 8,168,425; PCT patent application publication NO.: WO2016/075473; the contents of each of which are incorporated herein by reference in their entirety.

### IV. Formulations

The compositions of the present invention may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, the formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the invention, the formulation may be selected from any of those taught, for example, in International Application PCT/US2012/069610, the contents of which are incorporated herein by reference in its entirety.

### Inactive Ingredients

In some embodiments, pharmaceutical or other formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present invention may be approved by the US Food and Drug Administration (FDA). A non-exhaustive list of inactive ingredients includes, 1,2,6-Hexanetriol, 1,2-Dimyristoyl-Sn-Glycero-3-(Phospho-S-(1-Glycerol)), 1,2-Dimyristoyl-Sn-Glycero-3-Phosphocholine, 1,2-Dioleoyl-Sn-Glycero-3-Phosphocholine, 1,2-Dipalmitoyl-Sn-Glycero-3-(Phospho-Rac-(1-Glycerol)), 1,2-Distearoyl-Sn-Glycero-3-(Phospho-Rac-(1-Glycerol)), 1,2-Distearoyl-Sn-Glycero-3-Phosphocholine, 1-O-Tolylbiguanide, 2-Ethyl-1,6-Hexanediol, Acetic Acid, Acetic Acid, Glacial, Acetic Anhydride, Acetone, Acetone Sodium Bisulfite, Acetylated Lanolin Alcohols, Acetylated Monoglycerides, Acetylcysteine, Acetyltryptophan, DL-, Acrylates Copolymer, Acrylic Acid-Isooctyl Acrylate Copolymer, Acrylic Adhesive 788, Activated Charcoal, Adcote 72A103, Adhesive Tape, Adipic Acid, Aerotex Resin 3730, Alanine, Albumin Aggregated, Albumin Colloidal, Albumin Human, Alcohol, Alcohol, Dehydrated, Alcohol, Denatured, Alcohol, Diluted, Alfadex, Alginic Acid, Alkyl Ammonium Sulfonic Acid Betaine, Alkyl Aryl Sodium Sulfonate, Allantoin, Allyl .Alpha.-Ionone, Almond Oil, Alpha.-Terpineol, Alpha.-Tocopherol, Alpha.-Tocopherol Acetate, Dl-, Alpha.-Tocopherol, Dl-, Aluminum Acetate, Aluminum Chlorohydroxy Allantoinate, Aluminum Hydroxide, Aluminum Hydroxide—Sucrose, Hydrated, Aluminum Hydroxide Gel, Aluminum Hydroxide Gel F 500, Aluminum Hydroxide Gel F 5000, Aluminum Monostearate, Aluminum Oxide, Aluminum Polyester, Aluminum Silicate, Aluminum Starch Octenylsuccinate, Aluminum Stearate, Aluminum Subacetate, Aluminum Sulfate Anhydrous, Amerchol C, Amerchol-Cab, Aminomethylpropanol, Ammonia, Ammonia Solution, Ammonia Solution, Strong, Ammonium Acetate, Ammonium Hydroxide, Ammonium Lauryl Sulfate, Ammonium Nonoxynol-4 Sulfate, Ammonium Salt Of C-12-C-15 Linear Primary Alcohol Ethoxylate, Ammonium Sulfate, Ammonyx, Amphoteric-2, Amphoteric-9, Anethole, Anhydrous Citric Acid, Anhydrous Dextrose, Anhydrous Lactose, Anhydrous Trisodium Citrate, Aniseed Oil, Anoxid Sbn, Antifoam, Antipyrine, Apaflurane, Apricot Kernel Oil Peg-6 Esters, Aquaphor, Arginine, Arlacel, Ascorbic Acid, Ascorbyl Palmitate, Aspartic Acid, Balsam Peru, Barium Sulfate, Beeswax, Beeswax, Synthetic, Beheneth-10, Bentonite, Benzalkonium Chloride, Benzenesulfonic Acid, Benzethonium Chloride, Benzododecinium Bromide, Benzoic Acid, Benzyl Alcohol, Benzyl Benzoate, Benzyl Chloride, Betadex, Bibapcitide, Bismuth Subgallate, Boric Acid, Brocrinat, Butane, Butyl Alcohol, Butyl Ester Of Vinyl Methyl Ether/Maleic Anhydride Copolymer (125000 Mw), Butyl Stearate, Butylated Hydroxyanisole, Butylated Hydroxytoluene, Butylene Glycol, Butylparaben, Butyric Acid, C20-40 Pareth-24, Caffeine, Calcium, Calcium Carbonate, Calcium Chloride, Calcium Gluceptate, Calcium Hydroxide, Calcium Lactate, Calcobutrol, Caldiamide Sodium, Caloxetate Trisodium, Calteridol Calcium, Canada Balsam, Caprylic/Capric Triglyceride, Caprylic/Capric/Stearic Triglyceride, Captan, Captisol, Caramel, Carbomer 1342, Carbomer 1382, Carbomer 934, Carbomer 934p, Carbomer 940, Carbomer 941, Carbomer 980, Carbomer 981, Carbomer Homopolymer Type B (Allyl Pentaerythritol Crosslinked), Carbomer Homopolymer Type C (Allyl Pentaerythritol Crosslinked), Carbon Dioxide, Carboxy Vinyl Copolymer, Carboxymethylcellulose, Carboxymethylcellulose Sodium, Carboxypolymethylene, Carrageenan, Carrageenan Salt, Castor Oil, Cedar Leaf Oil, Cellulose, Cellulose, Microcrystalline, Cerasynt-Se, Ceresin, Ceteareth-12, Ceteareth-15, Ceteareth-30, Cetearyl Alcohol/Ceteareth-20, Cetearyl Ethylhexanoate, Ceteth-10, Ceteth-2, Ceteth-20, Ceteth-23, Cetostearyl Alcohol, Cetrimonium Chloride, Cetyl Alcohol, Cetyl Esters Wax, Cetyl Palmitate, Cetylpyridinium Chloride, Chlorobutanol, Chlorobutanol Hemihydrate, Chlorobutanol, Anhydrous, Chlorocresol, Chloroxylenol, Cholesterol, Choleth, Choleth-24, Citrate, Citric Acid, Citric Acid Monohydrate, Citric Acid, Hydrous, Cocamide Ether Sulfate, Cocamine Oxide, Coco Betaine, Coco Diethanolamide, Coco Monoethanolamide, Cocoa Butter, Coco-Glycerides, Coconut Oil, Coconut Oil, Hydrogenated, Coconut Oil/Palm Kernel Oil Glycerides, Hydrogenated, Cocoyl Caprylocaprate, Cola Nitida Seed Extract, Collagen, Coloring Suspension, Corn Oil, Cottonseed Oil, Cream Base, Creatine, Creatinine, Cresol, Croscarmellose Sodium, Crospovidone, Cupric Sulfate, Cupric Sulfate Anhydrous, Cyclomethicone, Cyclomethicone/Dimethicone Copolyol, Cysteine, Cysteine Hydrochloride, Cysteine Hydrochloride Anhydrous, Cysteine, Dl-, D&C Red No. 28, D&C Red No. 33, D&C Red No. 36, D&C Red No. 39, D&C Yellow No. 10, Dalfampridine, Daubert 1-5 Pestr (Matte) 164z, Decyl Methyl Sulfoxide, Dehydag Wax Sx, Dehydroacetic Acid, Dehymuls E, Denatonium Benzoate, Deoxycholic Acid, Dextran, Dextran 40, Dextrin, Dextrose, Dextrose Monohydrate, Dextrose Solution, Diatrizoic Acid, Diazolidinyl Urea, Dichlorobenzyl Alcohol, Dichlorodifluoromethane, Dichlorotetrafluoroethane, Diethanolamine, Diethyl Pyrocarbonate, Diethyl Sebacate, Diethylene Glycol Monoethyl Ether, Diethylhexyl Phthalate, Dihydroxyaluminum Aminoacetate, Diisopropanolamine, Diisopropyl Adipate, Diisopropyl Dilinoleate, Dimethicone 350, Dimethicone Copolyol, Dimethicone Mdx4-4210, Dimethicone Medical Fluid 360, Dimethyl Isosorbide, Dimethyl Sulfoxide, Dimethylaminoethyl Methacrylate-Butyl Methacrylate-Methyl Methacrylate Copolymer, Dimethyldioctadecylammonium Bentonite, Dimethylsiloxane/Methylvinylsiloxane Copolymer, Dinoseb Ammonium Salt, Dipalmitoylphosphatidylglycerol, Dl-, Dipropylene Glycol, Disodium Cocoamphodiacetate, Disodium Laureth Sulfosuccinate, Disodium Lauryl Sulfosuccinate, Disodium Sulfosalicylate, Disofenin, Divinylbenzene Styrene Copolymer, Dmdm Hydantoin, Docosanol, Docusate Sodium, Duro-Tak 280-2516, Duro-Tak 387-2516, Duro-Tak 80-1196, Duro-Tak 87-2070, Duro-Tak 87-2194, Duro-Tak 87-2287, Duro-Tak 87-2296, Duro-Tak 87-2888, Duro-Tak 87-2979, Edetate Calcium Disodium, Edetate Disodium, Edetate Disodium Anhydrous, Edetate Sodium, Edetic Acid, Egg Phospholipids, Entsufon, Entsufon Sodium, Epilactose, Epitetracycline Hydrochloride, Essence Bouquet 9200, Ethanolamine Hydrochloride, Ethyl Acetate, Ethyl Oleate, Ethylcelluloses, Ethylene Glycol, Ethylene Vinyl Acetate Copolymer, Ethylenediamine, Ethylenediamine Dihydrochloride, Ethylene-Propylene Copolymer, Ethylene-Vinyl Acetate Copolymer (28% Vinyl Acetate), Ethylene-Vinyl Acetate Copolymer (9% Vinylacetate), Ethylhexyl Hydroxystearate, Ethylparaben, Eucalyptol, Exametazime, Fat, Edible, Fat, Hard, Fatty Acid Esters, Fatty Acid Pentaerythriol Ester, Fatty Acids, Fatty Alcohol Citrate, Fatty Alcohols, Fd&C Blue No. 1, Fd&C Green No. 3, Fd&C Red No. 4, Fd&C Red No. 40, Fd&C Yellow No. 10 (Delisted), Fd&C Yellow No. 5, Fd&C Yellow No. 6, Ferric Chloride, Ferric Oxide, Flavor 89-186, Flavor 89-259, Flavor Df-119, Flavor Df-1530, Flavor Enhancer, Flavor Fig 827118, Flavor Raspberry Pfc-8407, Flavor Rhodia Pharmaceutical No. Rf 451, Fluorochlorohydrocarbons, Formaldehyde, Formaldehyde Solution, Fractionated Coconut Oil, Fragrance 3949-5, Fragrance 520a, Fragrance 6.007, Fragrance 91-122, Fragrance 9128-Y, Fragrance 93498g, Fragrance Balsam Pine No. 5124, Fragrance Bouquet 10328, Fragrance Chemoderm 6401-B, Fragrance Chemoderm 6411, Fragrance Cream No. 73457, Fragrance Cs-28197, Fragrance Felton 066m, Fragrance Firmenich 47373, Fragrance Givaudan Ess 9090/1c, Fragrance H-6540, Fragrance Herbal 10396, Fragrance Nj-1085, Fragrance P O Fl-147, Fragrance Pa 52805, Fragrance Pera Derm D, Fragrance Rbd-9819, Fragrance Shaw Mudge U-7776, Fragrance Tf 044078, Fragrance Ungerer Honeysuckle K 2771, Fragrance Ungerer N5195, Fructose, Gadolinium Oxide, Galactose, Gamma Cyclodextrin, Gelatin, Gelatin, Crosslinked, Gelfoam Sponge, Gellan Gum (Low Acyl), Gelva 737, Gentisic Acid, Gentisic Acid Ethanolamide, Gluceptate Sodium, Gluceptate Sodium Dihydrate, Gluconolactone, Glucuronic Acid, Glutamic Acid, Dl-, Glutathione, Glycerin, Glycerol Ester Of Hydrogenated Rosin, Glyceryl Citrate, Glyceryl Isostearate, Glyceryl Laurate, Glyceryl Monostearate, Glyceryl Oleate, Glyceryl Oleate/Propylene Glycol, Glyceryl Palmitate, Glyceryl Ricinoleate, Glyceryl Stearate, Glyceryl Stearate—Laureth-23, Glyceryl Stearate/Peg Stearate, Glyceryl Stearate/Peg-100 Stearate, Glyceryl Stearate/Peg-40 Stearate, Glyceryl Stearate-Stearamidoethyl Diethylamine, Glyceryl Trioleate, Glycine, Glycine Hydrochloride, Glycol Distearate, Glycol Stearate, Guanidine Hydrochloride, Guar Gum, Hair Conditioner (18n195-1m), Heptane, Hetastarch, Hexylene Glycol, High Density Polyethylene, Histidine, Human Albumin Microspheres, Hyaluronate Sodium, Hydrocarbon, Hydrocarbon Gel, Plasticized, Hydrochloric Acid, Hydrochloric Acid, Diluted, Hydrocortisone, Hydrogel Polymer, Hydrogen Peroxide, Hydrogenated Castor Oil, Hydrogenated Palm Oil, Hydrogenated Palm/Palm Kernel Oil Peg-6 Esters, Hydrogenated Polybutene 635-690, Hydroxide Ion, Hydroxyethyl Cellulose, Hydroxyethylpiperazine Ethane Sulfonic Acid, Hydroxymethyl Cellulose, Hydroxyoctacosanyl Hydroxystearate, Hydroxypropyl Cellulose, Hydroxypropyl Methylcellulose 2906, Hydroxypropyl-Bcyclodextrin, Hypromellose 2208 (15000 Mpa·S), Hypromellose 2910 (15000 Mpa·S), Hypromelloses, Imidurea, Iodine, Iodoxamic Acid, Iofetamine Hydrochloride, Irish Moss Extract, Isobutane, Isoceteth-20, Isoleucine, Isooctyl Acrylate, Isopropyl Alcohol, Isopropyl Isostearate, Isopropyl Myristate, Isopropyl Myristate—Myristyl Alcohol, Isopropyl Palmitate, Isopropyl Stearate, Isostearic Acid, Isostearyl Alcohol, Isotonic Sodium Chloride Solution, Jelene, Kaolin, Kathon Cg, Kathon Cg II, Lactate, Lactic Acid, Lactic Acid, Dl-, Lactic Acid, L-, Lactobionic Acid, Lactose, Lactose Monohydrate, Lactose, Hydrous, Laneth, Lanolin, Lanolin Alcohol—Mineral Oil, Lanolin Alcohols, Lanolin Anhydrous, Lanolin Cholesterols, Lanolin Nonionic Derivatives, Lanolin, Ethoxylated, Lanolin, Hydrogenated, Lauralkonium Chloride, Lauramine Oxide, Laurdimonium Hydrolyzed Animal Collagen, Laureth Sulfate, Laureth-2, Laureth-23, Laureth-4, Lauric Diethanolamide, Lauric Myristic Diethanolamide, Lauroyl Sarcosine, Lauryl Lactate, Lauryl Sulfate, Lavandula angustifolia Flowering Top, Lecithin, Lecithin Unbleached, Lecithin, Egg, Lecithin, Hydrogenated, Lecithin, Hydrogenated Soy, Lecithin, Soybean, Lemon Oil, Leucine, Levulinic Acid, Lidofenin, Light Mineral Oil, Light Mineral Oil (85 Ssu), Limonene, (+/−)-, Lipocol Sc-15, Lysine, Lysine Acetate, Lysine Monohydrate, Magnesium Aluminum Silicate, Magnesium Aluminum Silicate Hydrate, Magnesium Chloride, Magnesium Nitrate, Magnesium Stearate, Maleic Acid, Mannitol, Maprofix, Mebrofenin, Medical Adhesive Modified 5-15, Medical Antiform A-F Emulsion, Medronate Disodium, Medronic Acid, Meglumine, Menthol, Metacresol, Metaphosphoric Acid, Methanesulfonic Acid, Methionine, Methyl Alcohol, Methyl Gluceth-10, Methyl Gluceth-20, Methyl Gluceth-20 Sesquistearate, Methyl Glucose Sesquistearate, Methyl Laurate, Methyl Pyrrolidone, Methyl Salicylate, Methyl Stearate, Methylboronic Acid, Methylcellulose (4000 Mpa·S), Methylcelluloses, Methylchloroisothiazolinone, Methylene Blue, Methylisothiazolinone, Methylparaben, Microcrystalline Wax, Mineral Oil, Mono And Diglyceride, Monostearyl Citrate, Monothioglycerol, Multisterol Extract, Myristyl Alcohol, Myristyl Lactate, Myristyl-.Gamma.-Picolinium Chloride, N-(Carbamoyl-Methoxy Peg-40)-1,2-Distearoyl-Cephalin Sodium, N,N-Dimethylacetamide, Niacinamide, Nioxime, Nitric Acid, Nitrogen, Nonoxynol Iodine, Nonoxynol-15, Nonoxynol-9, Norflurane, Oatmeal, Octadecene-1/Maleic Acid Copolymer, Octanoic Acid, Octisalate, Octoxynol-1, Octoxynol-40, Octoxynol-9, Octyldodecanol, Octylphenol Polymethylene, Oleic Acid, Oleth-10/Oleth-5, Oleth-2, Oleth-20, Oleyl Alcohol, Oleyl Oleate, Olive Oil, Oxidronate Disodium, Oxyquinoline, Palm Kernel Oil, Palmitamine Oxide, Parabens, Paraffin, Paraffin, White Soft, Parfum Creme 45/3, Peanut Oil, Peanut Oil, Refined, Pectin, Peg 6-32 Stearate/Glycol Stearate, Peg Vegetable Oil, Peg-100 Stearate, Peg-12 Glyceryl Laurate, Peg-120 Glyceryl Stearate, Peg-120 Methyl Glucose Dioleate, Peg-15 Cocamine, Peg-150 Distearate, Peg-2 Stearate, Peg-20 Sorbitan Isostearate, Peg-22 Methyl Ether/Dodecyl Glycol Copolymer, Peg-25 Propylene Glycol Stearate, Peg-4 Dilaurate, Peg-4 Laurate, Peg-40 Castor Oil, Peg-40 Sorbitan Diisostearate, Peg-45/Dodecyl Glycol Copolymer, Peg-5 Oleate, Peg-50 Stearate, Peg-54 Hydrogenated Castor Oil, Peg-6 Isostearate, Peg-60 Castor Oil, Peg-60 Hydrogenated Castor Oil, Peg-7 Methyl Ether, Peg-75 Lanolin, Peg-8 Laurate, Peg-8 Stearate, Pegoxol 7 Stearate, Pentadecalactone, Pentaerythritol Cocoate, Pentasodium Pentetate, Pentetate Calcium Trisodium, Pentetic Acid, Peppermint Oil, Perflutren, Perfume 25677, Perfume Bouquet, Perfume E-1991, Perfume Gd 5604, Perfume Tana 90/42 Scba, Perfume W-1952-1, Petrolatum, Petrolatum, White, Petroleum Distillates, Phenol, Phenol, Liquefied, Phenonip, Phenoxyethanol, Phenylalanine, Phenylethyl Alcohol, Phenylmercuric Acetate, Phenylmercuric Nitrate, Phosphatidyl Glycerol, Egg, Phospholipid, Phospholipid, Egg, Phospholipon 90g, Phosphoric Acid, Pine Needle Oil (Pinus sylvestris), Piperazine Hexahydrate, Plastibase-50w, Polacrilin, Polidronium Chloride, Poloxamer 124, Poloxamer 181, Poloxamer 182, Poloxamer 188, Poloxamer 237, Poloxamer 407, Poly(Bis(P-Carboxyphenoxy)Propane Anhydride):Sebacic Acid, Poly(Dimethylsiloxane/Methylvinylsiloxane/Methylhydrogensiloxane) Dimethylvinyl Or Dimethylhydroxy Or Trimethyl Endblocked, Poly(Dl-Lactic-Co-Glycolic Acid), (50:50, Poly(Dl-Lactic-Co-Glycolic Acid), Ethyl Ester Terminated, (50:50, Polyacrylic Acid (250000 Mw), Polybutene (1400 Mw), Polycarbophil, Polyester, Polyester Polyamine Copolymer, Polyester Rayon, Polyethylene Glycol 1000, Polyethylene Glycol 1450, Polyethylene Glycol 1500, Polyethylene Glycol 1540, Polyethylene Glycol 200, Polyethylene Glycol 300, Polyethylene Glycol 300-1600, Polyethylene Glycol 3350, Polyethylene Glycol 400, Polyethylene Glycol 4000, Polyethylene Glycol 540, Polyethylene Glycol 600, Polyethylene Glycol 6000, Polyethylene Glycol 8000, Polyethylene Glycol 900, Polyethylene High Density Containing Ferric Oxide Black (<1%), Polyethylene Low Density Containing Barium Sulfate (20-24%), Polyethylene T, Polyethylene Terephthalates, Polyglactin, Polyglyceryl-3 Oleate, Polyglyceryl-4 Oleate, Polyhydroxyethyl Methacrylate, Polyisobutylene, Polyisobutylene (1100000 Mw), Polyisobutylene (35000 Mw), Polyisobutylene 178-236, Polyisobutylene 241-294, Polyisobutylene 35-39, Polyisobutylene Low Molecular Weight, Polyisobutylene Medium Molecular Weight, Polyisobutylene/Polybutene Adhesive, Polylactide, Polyols, Polyoxyethylene—Polyoxypropylene 1800, Polyoxyethylene Alcohols, Polyoxyethylene Fatty Acid Esters, Polyoxyethylene Propylene, Polyoxyl 20 Cetostearyl Ether, Polyoxyl 35 Castor Oil, Polyoxyl 40 Hydrogenated Castor Oil, Polyoxyl 40 Stearate, Polyoxyl 400 Stearate, Polyoxyl 6 And Polyoxyl 32 Palmitostearate, Polyoxyl Distearate, Polyoxyl Glyceryl Stearate, Polyoxyl Lanolin, Polyoxyl Palmitate, Polyoxyl Stearate, Polypropylene, Polypropylene Glycol, Polyquaternium-10, Polyquaternium-7 (70/30 Acrylamide/Dadmac, Polysiloxane, Polysorbate 20, Polysorbate 40, Polysorbate 60, Polysorbate 65, Polysorbate 80, Polyurethane, Polyvinyl Acetate, Polyvinyl Alcohol, Polyvinyl Chloride, Polyvinyl Chloride-Polyvinyl Acetate Copolymer, Polyvinylpyridine, Poppy Seed Oil, Potash, Potassium Acetate, Potassium Alum, Potassium Bicarbonate, Potassium Bisulfite, Potassium Chloride, Potassium Citrate, Potassium Hydroxide, Potassium Metabisulfite, Potassium Phosphate, Dibasic, Potassium Phosphate, Monobasic, Potassium Soap, Potassium Sorbate, Povidone Acrylate Copolymer, Povidone Hydrogel, Povidone K17, Povidone K25, Povidone K29/32, Povidone K30, Povidone K90, Povidone K90f, Povidone/Eicosene Copolymer, Povidones, Ppg-12/Smdi Copolymer, Ppg-15 Stearyl Ether, Ppg-20 Methyl Glucose Ether Distearate, Ppg-26 Oleate, Product Wat, Proline, Promulgen D, Promulgen G, Propane, Propellant A-46, Propyl Gallate, Propylene Carbonate, Propylene Glycol, Propylene Glycol Diacetate, Propylene Glycol Dicaprylate, Propylene Glycol Monolaurate, Propylene Glycol Monopalmitostearate, Propylene Glycol Palmitostearate, Propylene Glycol Ricinoleate, Propylene Glycol/Diazolidinyl Urea/Methylparaben/Propylparben, Propylparaben, Protamine Sulfate, Protein Hydrolysate, Pvm/Ma Copolymer, Quaternium-15, Quaternium-15 Cis-Form, Quaternium-52, Ra-2397, Ra-3011, Saccharin, Saccharin Sodium, Saccharin Sodium Anhydrous, Safflower Oil, Sd Alcohol 3a, Sd Alcohol 40, Sd Alcohol 40-2, Sd Alcohol 40b, Sepineo P 600, Serine, Sesame Oil, Shea Butter, Silastic Brand Medical Grade Tubing, Silastic Medical Adhesive, Silicone Type A, Silica, Dental, Silicon, Silicon Dioxide, Silicon Dioxide, Colloidal, Silicone, Silicone Adhesive 4102, Silicone Adhesive 4502, Silicone Adhesive Bio-Psa Q7-4201, Silicone Adhesive Bio-Psa Q7-4301, Silicone Emulsion, Silicone/Polyester Film Strip, Simethicone, Simethicone Emulsion, Sipon Ls 20np, Soda Ash, Sodium Acetate, Sodium Acetate Anhydrous, Sodium Alkyl Sulfate, Sodium Ascorbate, Sodium Benzoate, Sodium Bicarbonate, Sodium Bisulfate, Sodium Bisulfite, Sodium Borate, Sodium Borate Decahydrate, Sodium Carbonate, Sodium Carbonate Decahydrate, Sodium Carbonate Monohydrate, Sodium Cetostearyl Sulfate, Sodium Chlorate, Sodium Chloride, Sodium Chloride Injection, Sodium Chloride Injection, Bacteriostatic, Sodium Cholesteryl Sulfate, Sodium Citrate, Sodium Cocoyl Sarcosinate, Sodium Desoxycholate, Sodium Dithionite, Sodium Dodecylbenzenesulfonate, Sodium Formaldehyde Sulfoxylate, Sodium Gluconate, Sodium Hydroxide, Sodium Hypochlorite, Sodium Iodide, Sodium Lactate, Sodium Lactate, L-, Sodium Laureth-2 Sulfate, Sodium Laureth-3 Sulfate, Sodium Laureth-5 Sulfate, Sodium Lauroyl Sarcosinate, Sodium Lauryl Sulfate, Sodium Lauryl Sulfoacetate, Sodium Metabisulfite, Sodium Nitrate, Sodium Phosphate, Sodium Phosphate Dihydrate, Sodium Phosphate, Dibasic, Sodium Phosphate, Dibasic, Anhydrous, Sodium Phosphate, Dibasic, Dihydrate, Sodium Phosphate, Dibasic, Dodecahydrate, Sodium Phosphate, Dibasic, Heptahydrate, Sodium Phosphate, Monobasic, Sodium Phosphate, Monobasic, Anhydrous, Sodium Phosphate, Monobasic, Dihydrate, Sodium Phosphate, Monobasic, Monohydrate, Sodium Polyacrylate (2500000 Mw), Sodium Pyrophosphate, Sodium Pyrrolidone Carboxylate, Sodium Starch Glycolate, Sodium Succinate Hexahydrate, Sodium Sulfate, Sodium Sulfate Anhydrous, Sodium Sulfate Decahydrate, Sodium Sulfite, Sodium Sulfosuccinated Undecyclenic Monoalkylolamide, Sodium Tartrate, Sodium Thioglycolate, Sodium Thiomalate, Sodium Thiosulfate, Sodium Thiosulfate Anhydrous, Sodium Trimetaphosphate, Sodium Xylenesulfonate, Somay 44, Sorbic Acid, Sorbitan, Sorbitan Isostearate, Sorbitan Monolaurate, Sorbitan Monooleate, Sorbitan Monopalmitate, Sorbitan Monostearate, Sorbitan Sesquioleate, Sorbitan Trioleate, Sorbitan Tristearate, Sorbitol, Sorbitol Solution, Soybean Flour, Soybean Oil, Spearmint Oil, Spermaceti, Squalane, Stabilized Oxychloro Complex, Stannous 2-Ethylhexanoate, Stannous Chloride, Stannous Chloride Anhydrous, Stannous Fluoride, Stannous Tartrate, Starch, Starch 1500, Pregelatinized, Starch, Corn, Stearalkonium Chloride, Stearalkonium Hectorite/Propylene Carbonate, Stearamidoethyl Diethylamine, Steareth-10, Steareth-100, Steareth-2, Steareth-20, Steareth-21, Steareth-40, Stearic Acid, Stearic Diethanolamide, Stearoxytrimethylsilane, Steartrimonium Hydrolyzed Animal Collagen, Stearyl Alcohol, Sterile Water For Inhalation, Styrene/Isoprene/Styrene Block Copolymer, Succimer, Succinic Acid, Sucralose, Sucrose, Sucrose Distearate, Sucrose Polyesters, Sulfacetamide Sodium, Sulfobutylether .Beta.-Cyclodextrin, Sulfur Dioxide, Sulfuric Acid, Sulfurous Acid, Surfactol Qs, Tagatose, D-, Talc, Tall Oil, Tallow Glycerides, Tartaric Acid, Tartaric Acid, Dl-, Tenox, Tenox-2, Tert-Butyl Alcohol, Tert-Butyl Hydroperoxide, Tert-Butylhydroquinone, Tetrakis(2-Methoxyisobutylisocyanide)Copper(I) Tetrafluoroborate, Tetrapropyl Orthosilicate, Tetrofosmin, Theophylline, Thimerosal, Threonine, Thymol, Tin, Titanium Dioxide, Tocopherol, Tocophersolan, Triacetin, Tricaprylin, Trichloromonofluoromethane, Trideceth-10, Triethanolamine Lauryl Sulfate, Trifluoroacetic Acid, Triglycerides, Medium Chain, Trihydroxystearin, Trilaneth-4 Phosphate, Trilaureth-4 Phosphate, Trisodium Citrate Dihydrate, Trisodium Hedta, Triton 720, Triton X-200, Trolamine, Tromantadine, Tromethamine, Tryptophan, Tyloxapol, Tyrosine, Undecylenic Acid, Union 76 Amsco-Res 6038, Urea, Valine, Vegetable Oil, Vegetable Oil Glyceride, Hydrogenated, Vegetable Oil, Hydrogenated, Versetamide, Viscarin, Viscose/Cotton, Vitamin E, Wax, Emulsifying, Wecobee Fs, White Ceresin Wax, White Wax, Xanthan Gum, Zinc, Zinc Acetate, Zinc Carbonate, Zinc Chloride, and/or Zinc Oxide.

### V. Dosing and Administration and Delivery

### Dosing

The present invention provides methods comprising administering any one or more or component of a biocircuit system to a subject in need thereof. These may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Administration

The pharmaceutical compositions, biocircuits, biocircuit components, effector modules including their SREs or payloads of the present invention may be administered by any route to achieve a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

### Parenteral and Injectable Administration

In some embodiments, pharmaceutical compositions, biocircuits, biocircuit components, effector modules including their SREs or payloads of the present invention may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, pharmaceutical compositions, biocircuits, biocircuit components, effector modules including their SREs or payloads of the present invention may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

In some embodiments, pharmaceutical compositions, biocircuits, biocircuit components, effector modules including their SREs or payloads of the present invention may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, pharmaceutical compositions, biocircuits, biocircuit components, effector modules including their SREs or payloads of the present invention may be formulated for administration topically. The skin may be an ideal target site for delivery as it is readily accessible. Three routes are commonly considered to deliver pharmaceutical compositions, biocircuits, biocircuit components, effector modules including their SREs or payloads of the present invention to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Pharmaceutical compositions, biocircuits, biocircuit components, effector modules including their SREs or payloads of the present invention can be delivered to the skin by several different approaches known in the art.

In some embodiments, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions, biocircuits, biocircuit components, effector modules including their SREs or payloads of the present invention described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present invention contemplates the use of transdermal patches, which often have the added advantage of providing controlled delivery of pharmaceutical compositions, biocircuits, biocircuit components, effector modules including their SREs or payloads of the present invention to the body. Such dosage forms may be prepared, for example, by dissolving and/or dispensing pharmaceutical compositions, biocircuits, biocircuit components, effector modules including their SREs or payloads in the proper medium. Alternatively, or additionally, rates may be controlled by either providing rate controlling membranes and/or by dispersing pharmaceutical compositions, biocircuits, biocircuit components, effector modules including their SREs or payloads in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, pharmaceutical compositions, biocircuits, biocircuit components, effector modules including their SREs or payloads of the present invention are formulated in depots for extended release. Generally, specific organs or tissues (“target tissues”) are targeted for administration.

In some aspects of the invention, pharmaceutical compositions, biocircuits, biocircuit components, effector modules including their SREs or payloads of the present invention are spatially retained within or proximal to target tissues. Provided are method of providing pharmaceutical compositions, biocircuits, biocircuit components, effector modules including their SREs or payloads to target tissues of mammalian subjects by contacting target tissues (which comprise one or more target cells) with pharmaceutical compositions, biocircuits, biocircuit components, effector modules including their SREs or payloads under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, biocircuits, biocircuit components, effector modules including their SREs or payloads that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions, biocircuits, biocircuit components, effector modules including their SREs or payloads administered to subjects are present intracellularly at a period of time following administration. For example, intramuscular injection to mammalian subjects may be performed using aqueous compositions comprising pharmaceutical compositions, biocircuits, biocircuit components, effector modules including their SREs or payloads of the present invention and one or more transfection reagent, and retention is determined by measuring the amount of pharmaceutical compositions, biocircuits, biocircuit components, effector modules including their SREs or payloads present in muscle cells.

Certain aspects of the invention are directed to methods of providing pharmaceutical compositions, biocircuits, biocircuit components, effector modules including their SREs or payloads of the present invention to a target tissues of mammalian subjects, by contacting target tissues (comprising one or more target cells) with pharmaceutical compositions, biocircuits, biocircuit components, effector modules including their SREs or payloads under conditions such that they are substantially retained in such target tissues. Pharmaceutical compositions, biocircuits, biocircuit components, effector modules including their SREs or payloads comprise enough active ingredient such that the effect of interest is produced in at least one target cell. In some embodiments, pharmaceutical compositions, biocircuits, biocircuit components, effector modules including their SREs or payloads generally comprise one or more cell penetration agents, although “naked” formulations (such as without cell penetration agents or other agents) are also contemplated, with or without pharmaceutically acceptable carriers.

In some embodiments, the amount of a growth factor present in cells in a tissue is desirably increased. Preferably, this increase in growth factor is spatially restricted to cells within the target tissue. Thus, provided are methods of increasing the amount of growth factor of interest in tissues of mammalian subjects. In some embodiments, formulations are provided comprising pharmaceutical compositions, biocircuits, biocircuit components, effector modules including their SREs or payloads characterized in that the unit quantity provided has been determined to produce a desired level of growth factor of interest in a substantial percentage of cells contained within predetermined volumes of target tissue.

In some embodiments, formulations comprise a plurality of different pharmaceutical compositions, biocircuits, biocircuit components, effector modules including their SREs or payloads, where one or more than one targets biomolecules of interest. Optionally, formulations may also comprise cell penetration agents to assist in the intracellular delivery of pharmaceutical compositions, biocircuits, biocircuit components, effector modules including their SREs or payloads. In such embodiments, determinations are made of compound and/or composition dose required to target biomolecules of interest in substantial percentages of cells contained within predetermined volumes of the target tissue (generally, without targeting biomolecules of interest in adjacent or distal tissues.) Determined doses are then introduced directly into subject tissues. In some embodiments, the invention provides for pharmaceutical compositions, biocircuits, biocircuit components, effector modules including their SREs or payloads to be delivered in more than one administration or by split dose administration.

### Pulmonary Administration

In some embodiments, pharmaceutical compositions, biocircuits, biocircuit components, effector modules including their SREs or payloads of the present invention may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

In some embodiments, pharmaceutical compositions, biocircuits, biocircuit components, effector modules including their SREs or payloads of the present invention may be administered nasally and/or intranasally. In some embodiments, formulations described herein as being useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Ophthalmic or Otic Administration

In some embodiments, pharmaceutical compositions, biocircuits, biocircuit components, effector modules including their SREs or payloads of the present invention may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Delivery

### Naked Delivery

Pharmaceutical compositions, biocircuits, biocircuit components, effector modules including their SREs or payloads of the present invention may be delivered to cells, tissues, organs and/or organisms in naked form. As used herein in, the term “naked” refers to pharmaceutical compositions, biocircuits, biocircuit components, effector modules including their SREs or payloads delivered free from agents or modifications which promote transfection or permeability. The naked pharmaceutical compositions, biocircuits, biocircuit components, effector modules including their SREs or payloads may be delivered to the cells, tissues, organs and/or organisms using routes of administration known in the art and described herein. In some embodiments, naked delivery may include formulation in a simple buffer such as saline or PBS.

### Formulated Delivery

In some embodiments, pharmaceutical compositions, biocircuits, biocircuit components, effector modules including their SREs or payloads of the present invention may be formulated, using methods described herein. Formulations may comprise pharmaceutical compositions, biocircuits, biocircuit components, effector modules including their SREs or payloads which may be modified and/or unmodified. Formulations may further include, but are not limited to, cell penetration agents, pharmaceutically acceptable carriers, delivery agents, bioerodible or biocompatible polymers, solvents, and/or sustained-release delivery depots. Formulations of the present invention may be delivered to cells using routes of administration known in the art and described herein.

pharmaceutical compositions, biocircuits, biocircuit components, effector modules including their SREs or payloads may also be formulated for direct delivery to organs or tissues in any of several ways in the art including, but not limited to, direct soaking or bathing, via a catheter, by gels, powder, ointments, creams, gels, lotions, and/or drops, by using substrates such as fabric or biodegradable materials coated or impregnated with compositions, and the like.

### Detectable Agents and Labels

The stimuli, biocircuit systems and components, effector modules including the SREs and payloads may be associated with or bound to one or more radioactive agents or detectable agents.

These agents include various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ⁸¹mKr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-l-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and-6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yl]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); TRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Kits

The present invention includes a variety of kits for conveniently and/or effectively carrying out methods of the present invention. Typically, kits will comprise sufficient amounts and/or numbers of components to allow a user to perform one or multiple treatments of a subject(s) and/or to perform one or multiple experiments.

In one embodiment, the present invention provides kits for inhibiting genes in vitro or in vivo, comprising a biocircuit of the present invention or a combination of biocircuits of the present invention, optionally in combination with any other suitable active agents.

The kit may further comprise packaging and instructions and/or a delivery agent to form a formulation composition. The delivery agent may comprise, for example, saline, a buffered solution.

In additional embodiments, assay screening kits are provided. The kit includes a container for the screening assay. An instruction for the use of the assay and the information about the screening method are to be included in the kit.

### VI. Delivery Modalities and/or Vectors

The biocircuit systems, effector modules, SREs and/or payloads of the present invention may be delivered using one or more modalities. The present invention also provides vectors that package polynucleotides of the invention encoding biocircuits, effector modules, SREs (DDs) and payload constructs, and combinations thereof. Vectors of the present invention may also be used to deliver the packaged polynucleotides to a cell, a local tissue site or a subject. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In general, vectors contain an origin of replication functional in at least one organism, a promoter sequence and convenient restriction endonuclease site, and one or more selectable markers e.g. a drug resistance gene.

As used herein a promoter is defined as a DNA sequence recognized by transcription machinery of the cell, required to initiate specific transcription of the polynucleotide sequence of the present invention. Vectors can comprise native or non-native promoters operably linked to the polynucleotides of the invention. The promoters selected may be strong, weak, constitutive, inducible, tissue specific, development stage-specific, and/or organism specific. One example of a suitable promoter is the immediate early cytomegalovirus (CMV) promoter. This promoter sequence is a strong constitutive promoter sequence capable of driving high levels of expression of polynucleotide sequence that is operatively linked to it. Another example of a preferred promoter is Elongation Growth Factor-1. Alpha (EF-1. alpha). Other constitutive promoters may also be used, including, but not limited to simian virus 40 (SV40), mouse mammary tumor virus (MMTV), human immunodeficiency virus (HIV), long terminal repeat (LTR), promoter, an avian leukemia virus promoter, an Epstein-Barr virus immediate early promoter, a Rous sarcoma virus promoter as well as human gene promoters including, but not limited to the phosphoglycerate kinase (PGK) promoter, actin promoter, the myosin promoter, the hemoglobin promoter, the Ubiquitin C (Ubc) promoter, the human U6 small nuclear protein promoter and the creatine kinase promoter. In some instances, inducible promoters such as but not limited to metallothionine promoter, glucocorticoid promoter, a progesterone promoter, and a tetracycline promoter may be used.

In some embodiments, the optimal promoter may be selected based on its ability to achieve minimal expression of the SREs and payloads of the invention in the absence of the ligand and detectable expression in the presence of the ligand.

Additional promoter elements e.g. enhancers may be used to regulate the frequency of transcriptional initiation. Such regions may be located 10-100 base pairs upstream or downstream of the start site. In some instances, two or more promoter elements may be used to cooperatively or independently activate transcription.

In some embodiments, the recombinant expression vector may comprise regulatory sequences, such as transcription and translation initiation and termination codons, which are specific to the type of host cell into which the vector is to be introduced.

In some embodiments, the vector of the invention may comprise one or more payloads taught herein, wherein the two or more payloads may be included in one effector module. In this case, the two or more payloads are tuned by the same stimulus simultaneously. In other embodiments, the vector of the invention may comprise two or more effector modules, wherein each effector module comprises a different payload. In this case, the two or more effector modules and payloads are tuned by different stimuli, providing separately independent regulation of the two or more components.

### Lentiviral Vehicles/Particles

In some embodiments, lentiviral vehicles/particles may be used as delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Biotechnol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. As used herein, the term “recombinant” refers to a vector or other nucleic acid containing both lentiviral sequences and non-lentiviral retroviral sequences.

Lentiviral particles may be generated by co-expressing the virus packaging elements and the vector genome itself in a producer cell such as human HEK293T cells. These elements are usually provided in three or four separate plasmids. The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the effector module of the present invention. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther., 2005, 11: 452-459), FreeStyle™ 293 Expression System (ThermoFisher, Waltham, Mass.), and other HEK293T-based producer cell lines (e.g., Stewart et al., Hum Gene Ther. 2011, 22(3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Other elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. The effector module is linked to the vector.

Methods for generating recombinant lentiviral particles are discussed in the art, for example, U.S. Pat. Nos. 8,846,385; 7,745,179; 7,629,153; 7,575,924; 7,179,903; and 6,808,905; the contents of each of which are incorporated herein by reference in their entirety.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII.

Lentiviral vehicles are plasmid-based or virus-based and are known in the art (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; the contents of each of which are incorporated herein by reference in their entirety).

### Adeno-Associated Viral Particles

Delivery of any of the biocircuits, biocircuit components, effector modules, SREs or payload constructs of the present invention may be achieved using recombinant adeno-associated viral (rAAV) vectors. Such vectors or viral particles may be designed to utilize any of the known serotype capsids or combinations of serotype capsids. Capsids may include but not limited to AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, and/or AAVrh.74.

In one embodiment, the AAV serotype may be or have a sequence as described in United States Publication No. US20030138772, herein incorporated by reference in its entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772) or variants thereof. Non limiting examples of variants include SEQ ID NOs: 9, 27-45, 47-62, 66-69, 73-81, 84-94, 96, 97, 99, 101-113 of US20030138772, the contents of which are herein incorporated by reference in its entirety.

In one embodiment, the AAV serotype may be or may have a sequence as described in United States Publication No. US20150159173, herein incorporated by reference in its entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In one embodiment, the AAV serotype may be or have the sequence as described in U.S. Pat. No. 7,198,951, herein incorporated by reference in its entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7).

In one embodiment, the AAV serotype may be or have a mutation in the AAV9 sequence as described by N Pulicherla et al. (Molcular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In one embodiment, the AAV serotype may be or have a sequence as described in U.S. Pat. No. 6,156,303, herein incorporated by reference in its entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In one embodiment, the AAV serotype may be or may have a sequence as described in United States Publication No. US20140359799, herein incorporated by reference in its entirety, such as, but not limited to, AAV8 (SEQ ID NO: 1 of US20140359799), AAVDJ (SEQ ID NO: 2 and 3 of US20140359799), or variants thereof.

In one embodiment, the AAV serotype may be or have the sequence of AAV4 as described in International Publication No. WO1998011244, herein incorporated by reference in its entirety, such as, but not limited to AAV4 (SEQ ID NO: 1-20 of WO1998011244).

In one embodiment, the AAV serotype may be or have a mutation in the AAV2 sequence to generate AAV2G9 as described in International Publication No. WO2014144229 and herein incorporated by reference in its entirety.

In one embodiment, the AAV serotype may be or have a sequence as described in International Publication WO2005033321, herein incorporated by reference in its entirety, such as, but not limited to AAV1 (SEQ ID NO: 202 and 219 of WO2005033321), AAV2 (SEQ ID NO: 211 and 221 of WO2005033321), AAV3-3 (SEQ ID NO: 200 and 217 of WO2005033321), AAV4-4 (SEQ ID NO: 201 and 218 of WO2005033321), AAV5 (SEQ ID NO: 216 and 199 of WO2005033321), AAV6 (SEQ ID NO: 203 and 220 of WO2005033321), AAV7 (SEQ ID NO: 213 and 222 of WO2005033321), AAV8 (SEQ ID NO: 214 and 223 of WO2005033321), hu.14/AAV9 (SEQ ID NO: 3 and 123 of WO2005033321), hu.17 (SEQ ID NO: 83 of WO2005033321), hu.6 (SEQ ID NO: 84 of WO2005033321), hu.42 (SEQ ID NO: 85 of WO2005033321), rh.38 (SEQ ID NO: 86 of WO2005033321), hu.40 (SEQ ID NO: 87 of WO2005033321), hu.37 (SEQ ID NO: 88 of WO2005033321), rh.40 (SEQ ID NO: 92 of WO2005033321), rh.52 (SEQ ID NO: 96 of WO2005033321), rh.53 (SEQ ID NO: 97 of WO2005033321), rh.49 (SEQ ID NO: 103 of WO2005033321), rh.51 (SEQ ID NO: 104 of WO2005033321), rh.57 (SEQ ID NO: 105 of WO2005033321), rh.58 (SEQ ID NO: 106 of WO2005033321), rh.61 (SEQ ID NO: 107 of WO2005033321), rh.50 (SEQ ID NO: 108 of WO2005033321), rh.43 (SEQ ID NO: 163 of WO2005033321), rh.62 (SEQ ID NO: 114 of WO2005033321), rh.48 (SEQ ID NO: 115 of WO2005033321), 4-9/rh.54 (SEQ ID NO: 116 of WO2005033321), 4-19/rh.55 (SEQ ID NO: 117 of WO2005033321), hu.31 (SEQ ID NO: 121 of WO2005033321), hu.32 (SEQ ID NO: 122 of WO2005033321), hu.34 (SEQ ID NO: 125 of WO2005033321), hu.45 (SEQ ID NO: 127 of WO2005033321), hu.47 (SEQ ID NO: 128 of WO2005033321), hu.13 (SEQ ID NO: 129 of WO2005033321), hu.28 (SEQ ID NO: 130 of WO2005033321), hu.29 (SEQ ID NO: 132 of WO2005033321), hu.19 (SEQ ID NO: 133 of WO2005033321), hu.20 (SEQ ID NO: 134 of WO2005033321), hu.21 (SEQ ID NO: 135 of WO2005033321), hu.23.2 (SEQ ID NO: 137 of WO2005033321), hu.22 (SEQ ID NO: 138 of WO2005033321), hu.27 (SEQ ID NO: 140 of WO2005033321), hu.4 (SEQ ID NO: 141 of WO2005033321), hu.2 (SEQ ID NO: 143 of WO2005033321), hu.1 (SEQ ID NO: 144 of WO2005033321), hu.3 (SEQ ID NO: 145 of WO2005033321), hu.25 (SEQ ID NO: 146 of WO2005033321), hu.15 (SEQ ID NO: 147 of WO2005033321), hu.16 (SEQ ID NO: 148 of WO2005033321), hu.18 (SEQ ID NO: 149 of WO2005033321), hu.7 (SEQ ID NO: 150 of WO2005033321), hu.11 (SEQ ID NO: 153 of WO2005033321), hu.9 (SEQ ID NO: 155 of WO2005033321), hu.10 (SEQ ID NO: 156 of WO2005033321), hu.48 (SEQ ID NO: 157 of WO2005033321), hu.44 (SEQ ID NO: 144 of WO2005033321), hu.46 (SEQ ID NO: 159 of WO2005033321), hu.43 (SEQ ID NO: 160 of WO2005033321), hu.35 (SEQ ID NO: 164 of WO2005033321), hu.24 (SEQ ID NO: 136 of WO2005033321), rh.64 (SEQ ID NO: 99 of WO2005033321), hu.41 (SEQ ID NO: 91 of WO2005033321), hu.39 (SEQ ID NO: 102 of WO2005033321), hu.67 (SEQ ID NO: 198 of WO2005033321), hu.66 (SEQ ID NO: 197 of WO2005033321), hu.51 (SEQ ID NO: 190 of WO2005033321), hu.52 (SEQ ID NO: 191 of WO2005033321), hu.49 (SEQ ID NO: 189 of WO2005033321), hu.56 (SEQ ID NO: 192 of WO2005033321), hu.57 (SEQ ID NO: 193 of WO2005033321), hu.58 (SEQ ID NO: 194 of WO2005033321), hu.63 (SEQ ID NO: 195 of WO2005033321), hu.64 (SEQ ID NO: 196), hu.60 (SEQ ID NO: 184 of WO2005033321), hu.61 (SEQ ID NO: 185 of WO2005033321), hu.53 (SEQ ID NO: 186 of WO2005033321), hu.55 (SEQ ID NO: 187 of WO2005033321), hu.54 (SEQ ID NO: 188 of WO2005033321), hu.6 (SEQ ID NO: 84 of WO2005033321), rh.56 (SEQ ID NO: 152 of WO2005033321), or variants thereof. Non limiting examples of variants include SEQ ID NOs: 1, 2, 4-82, 89, 90, 93-95, 98, 100, 101, 109-113, 118-120, 124, 126, 131, 139, 142, 151,154, 158, 161, 162, 165-183, 202, 204-212, 215, 219, 224-236 of WO2005033321, the contents of which are herein incorporated by reference in its entirety.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The biocircuits, biocircuit components, effector modules, SREs or payload constructs may be encoded in one or more viral genomes to be packaged in the AAV capsids taught herein.

Such vector or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

The biocircuits, biocircuit components, effector modules, SREs or payload constructs of the invention may be administered in one or more AAV particles.

In some embodiments, the effector modules may be administered in one or more AAV particles. In some embodiments, more than one effector module or SRE may be encoded in a viral genome.

### Retroviral Vehicles/Particles (γ-Retroviral Vectors)

In some embodiments, retroviral vehicles/particles may be used to deliver the biocircuits, biocircuit components, effector modules, SREs or payload constructs of the present invention. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xpr1) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present invention that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelop proteins include the gibbon ape leukemia virus envelope protein (GALV) or vesicular stomatitis virus G protein (VSV-G), or Simian endogenous retrovirus envelop protein, or Measles Virus H and F proteins, or Human immunodeficiency virus gp120 envelop protein, or cocal vesiculovirus envelop protein (See, e.g., U.S. application publication NO.: 2012/164118; the contents of which are incorporated herein by reference in its entirety). In other aspects, envelope glycoproteins may be genetically modified to incorporate targeting/binding ligands into gamma-retroviral vectors, binding ligands including, but not limited to, peptide ligands, single chain antibodies and growth factors (Waehler et al., Nat. Rev. Genet. 2007, 8(8):573-587; the contents of which are incorporated herein by reference in its entirety). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al., Biotechnol. Bioeng., 2008, 101(2): 357-368; and Maetzig et al., Viruses, 2011, 3, 677-713; the contents of each of which are incorporated herein by reference in their entirety).

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promotor of choice, and/or an enhancer element. The choice of the internal promotors may be made according to specific requirements of gene expression needed for a particular purpose of the invention.

In some embodiments, polynucleotides encoding the biocircuit, biocircuit components, effector module, SRE are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Gammaretroviral vectors suitable for delivering biocircuit components, effector modules, SREs or payload constructs of the present invention may be selected from those disclosed in U.S. Pat. Nos. 8,828,718; 7,585,676; 7,351,585; U.S. application publication NO.: 2007/048285; PCT application publication NOs.: WO2010/113037; WO2014/121005; WO2015/056014; and EP Pat. NOs.: EP1757702; EP1757703 (the contents of each of which are incorporated herein by reference in their entirety).

### Oncolytic Viral Vector

In some embodiments, polynucleotides of present invention may be packaged into oncolytic viruses. As used herein, the term “oncolytic virus” refers to a virus that preferentially infects and kills cancer cells such as vaccine viruses. An oncolytic virus can occur naturally or can be a genetically modified virus such as oncolytic adenovirus, and oncolytic herpes virus.

In some embodiments, oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor; See e.g., U.S. Pat. No. 9,226,977; the contents of which are incorporated herein by reference in their entirety.

Messenger RNA (mRNA)

In some embodiments, the effector modules of the invention may be designed as a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Application number PCT/US2013/030062, the contents of which are incorporated herein by reference in its entirety.

Polynucleotides of the invention may also be designed as taught in, for example, Ribostem Limited in United Kingdom patent application serial number 0316089.2 filed on Jul. 9, 2003 now abandoned, PCT application number PCT/GB2004/002981 filed on Jul. 9, 2004 published as WO2005005622, U.S. patent application national phase entry Ser. No. 10/563,897 filed on Jun. 8, 2006 published as US20060247195 now abandoned, and European patent application national phase entry serial number EP2004743322 filed on Jul. 9, 2004 published as EP1646714 now withdrawn; Novozymes, Inc. in PCT application number PCT/US2007/88060 filed on Dec. 19, 2007 published as WO2008140615, United States patent application national phase entry Ser. No. 12/520,072 filed on Jul. 2, 2009 published as US20100028943 and European patent application national phase entry serial number EP2007874376 filed on Jul. 7, 2009 published as EP2104739; University of Rochester in PCT application number PCT/US2006/46120 filed on Dec. 4, 2006 published as WO2007064952 and U.S. patent application Ser. No. 11/606,995 filed on Dec. 1, 2006 published as US20070141030; BioNTech AG in European patent application serial number EP2007024312 filed Dec. 14, 2007 now abandoned, PCT application number PCT/EP2008/01059 filed on Dec. 12, 2008 published as WO2009077134, European patent application national phase entry serial number EP2008861423 filed on Jun. 2, 2010 published as EP2240572, United States patent application national phase entry Ser. No. 12/735,060 filed Nov. 24, 2010 published as US20110065103, German patent application serial number DE 10 2005 046 490 filed Sep. 28, 2005, PCT application PCT/EP2006/0448 filed Sep. 28, 2006 published as WO2007036366, national phase European patent EP1934345 published Mar. 21, 2012 and national phase U.S. patent application Ser. No. 11/992,638 filed Aug. 14, 2009 published as 20100129877; Immune Disease Institute Inc. in U.S. patent application Ser. No. 13/088,009 filed Apr. 15, 2011 published as US20120046346 and PCT application PCT/US2011/32679 filed Apr. 15, 2011 published as WO20110130624; Shire Human Genetic Therapeutics in U.S. patent application Ser. No. 12/957,340 filed on Nov. 20, 2010 published as US20110244026; Sequitur Inc. in PCT application PCT/US1998/019492 filed on Sep. 18, 1998 published as WO1999014346; The Scripps Research Institute in PCT application number PCT/US2010/00567 filed on Feb. 24, 2010 published as WO2010098861, and United States patent application national phase entry Ser. No. 13/203,229 filed Nov. 3, 2011 published as US20120053333; Ludwig-Maximillians University in PCT application number PCT/EP2010/004681 filed on Jul. 30, 2010 published as WO2011012316; Cellscript Inc. in U.S. Pat. No. 8,039,214 filed Jun. 30, 2008 and granted Oct. 18, 2011, U.S. patent application Ser. No. 12/962,498 filed on Dec. 7, 2010 published as US20110143436, Ser. No. 12/962,468 filed on Dec. 7, 2010 published as US20110143397, Ser. No. 13/237,451 filed on Sep. 20, 2011 published as US20120009649, and PCT applications PCT/US2010/59305 filed Dec. 7, 2010 published as WO2011071931 and PCT/US2010/59317 filed on Dec. 7, 2010 published as WO2011071936; The Trustees of the University of Pennsylvania in PCT application number PCT/US2006/32372 filed on Aug. 21, 2006 published as WO2007024708, and U.S. patent application national phase entry Ser. No. 11/990,646 filed on Mar. 27, 2009 published as US20090286852; Curevac GMBH in German patent application serial numbers DE10 2001 027 283.9 filed Jun. 5, 2001, DE10 2001 062 480.8 filed Dec. 19, 2001, and DE 20 2006 051 516 filed Oct. 31, 2006 all abandoned, European patent numbers EP1392341 granted Mar. 30, 2005 and EP1458410 granted Jan. 2, 2008, PCT application numbers PCT/EP2002/06180 filed Jun. 5, 2002 published as WO2002098443, PCT/EP2002/14577 filed on Dec. 19, 2002 published as WO2003051401, PCT/EP2007/09469 filed on Dec. 31, 2007 published as WO2008052770, PCT/EP2008/03033 filed on Apr. 16, 2008 published as WO2009127230, PCT/EP2006/004784 filed on May 19, 2005 published as WO2006122828, PCT/EP2008/00081 filed on Jan. 9, 2007 published as WO2008083949, and U.S. patent application Ser. No. 10/729,830 filed on Dec. 5, 2003 published as US20050032730, Ser. No. 10/870,110 filed on Jun. 18, 2004 published as US20050059624, Ser. No. 11/914,945 filed on Jul. 7, 2008 published as US20080267873, Ser. No. 12/446,912 filed on Oct. 27, 2009 published as US2010047261 now abandoned, Ser. No. 12/522,214 filed on Jan. 4, 2010 published as US20100189729, Ser. No. 12/787,566 filed on May 26, 2010 published as US20110077287, Ser. No. 12/787,755 filed on May 26, 2010 published as US20100239608, Ser. No. 13/185,119 filed on Jul. 18, 2011 published as US20110269950, and Ser. No. 13/106,548 filed on May 12, 2011 published as US20110311472 all of which are herein incorporated by reference in their entirety.

In some embodiments, the effector modules may be designed as self amplifying RNA. “Self amplifying RNA” as used herein refers to RNA molecules that can replicate in the host resulting in the increase in the amount of the RNA and the protein encoded by the RNA. Such self amplifying RNA may have structural features or components of any of those taught in International Patent Application Publication No. WO2011005799 (the contents of which are incorporated herein by reference in their entirety.)

### VII. Methods and Uses

The biocircuits, effector modules, SREs, stimuli, compositions or systems comprising one or more of the stimuli, biocircuits, effector modules of the present invention may be utilized in a large variety of applications including, but not limited to, therapeutics, diagnosis and prognosis, bioengineers, bioprocessing, biofactory, research agents, metabolomics, gene expression, enzyme replacement, etc.

### Microbiome

The biocircuits of the present invention and/or any of their components may be utilized in regulating or tuning the microbiome. A diverse community of symbiotic, commensal and pathogenic microorganisms exist in all environmentally exposed sites in the body and is herein referred to as the “Microbiome.” Environmentally exposed sites of the body that may be inhabited by a microbiome include the skin, nasopharynx, the oral cavity, respiratory tract, gastrointestinal tract, and the reproductive tract. The intimate association of the microbiome with the body has profound implications on human health and disease, including asthma, inflammatory bowel diseases, metabolic, cardiovascular diseases and cancer. Accordingly, in some embodiments, biocircuits, biocircuit components, effector modules including their SREs or payloads of the present invention may be used in the modulation or alteration or exploitation of the microbiome and/or the microenvironment of the microbiome.

In some embodiments, biocircuits of the present invention are triggered by one or more stimuli within the microbiome or the microenvironment of the microbiome. In one embodiment, such stimuli may be related to a disease state in the host.

In some embodiments, biocircuits of the present invention may be used in treating dysbiosis. As used herein “dysbiosis refers to the disequilibrium between potentially detrimental and know beneficial microorganisms within the microbiome. As a non limiting example, biocircuits of the present invention may be used to treat obesity and metabolic disorders. Patients with such conditions have higher levels of Firmicutes phylum and lower levels of Escherichia coli in their feces. Increased proportions of E. coli in these patients has been associated with weight loss and lower levels of serum leptin.

In some embodiments, one or microbial proteins, RNA and/or other biomolecules may be utilized as payloads in the microbiome. As a non limiting example, the payload may be NAPE synthase, an enzyme that is responsible for the synthesis of N-acyl-phosphatidylethanolamine (NAPE). Transplantation of Escherichia coli engineered to express NAPE synthase into host organisms can prevent obesity by inducing satiety and reducing food intake (International Publication No. WO2013043719; the contents of which are incorporated by reference in their entirety).

In some embodiments, the microbiome may be engineered with biocircuits consisting of non-microbial biomolecules as payloads. Non limiting examples of payloads include, antiviral peptide, enzymes, neuropeptides, cytokines, and other soluble factors. Such strategies transform the microbiome into therapeutic agents for the treatment of diseases. As a non limiting example, Glucagon like peptide-1 may be used as a payload. Administration of Lactobacillus gasseri engineered to express Glucagon like peptide-1, induced insulin production in the host and decreased hyperglycemia (Duan, F., et al., Diabetes, 64, 1794-1803 (2015); the contents of which are incorporated by reference in their entirety).

In some embodiments, the microbiome may comprise a kill switch. As used herein the term “kill switch” refers to biocircuits of the present invention that include one or more toxins as a payload. Microorganisms engineered for in vivo administration may be programmed to die at a specific time, after the delivery of gene or genes, and/or after the host has experienced the therapeutic effect. Specifically, it may be useful to prevent long-term colonization of the host by the organisms or spread of the microorganisms outside the area of interest. Examples of toxins that can be used in kill switches include, but are not limited to, bacteriocins, lysins, and other molecules that cause cell death by lysing cell membranes, degrading cellular DNA, or other mechanisms.

Alterations in the composition of the microbiome may impact the action of anti-cancer therapies. Lida et al., have demonstrated that mice with intact microbiome are far more responsive to Oxaliplatin-based chemotherapy than germ-free mice (Iida N., et al. Commensal bacteria control cancer response to therapy by modulating the tumor microenvironment. Science, 2013; 342: 967-70; the contents of which are incorporated by reference in their entirety). Microbiome composition also appears to be critical to elicit the beneficial effect. For example, Parabacteroides distasonis can dampen the therapeutic effects of cyclophosphamide therapy (Viaud S., et al., The intestinal microbiota modulates the anticancer immune effects of cyclophosphamide. Science, 2013; 342: 971-6). Thus in some embodiments, microbiome engineered with the biocircuits of the present invention may be used to improve the efficacy of the anti-cancer therapies.

In some embodiments, microbiome engineered with the biocircuits of the present invention may be used to improve the efficacy of the anti-cancer immunotherapies. Sivan et al., found that mice with Bifidobacterium in their gut microbiome were more responsive to immune check point blockage e.g. anti PD-L1 immunotherapy in subcutaneous melanoma tumor model (Sivan A., et al. Commensal Bifidobacterium promotes antitumor immunity and facilitates anti-PD-L1 efficacy. Science 2015; 350:1084-9; the contents of which are incorporated by reference in their entirety). In one embodiment, protein, RNA and/or other biomolecules derived from the microbiome may be used as payload to influence the efficacy of the anti-cancer immunotherapies.

### Metabolite Biosensors

In some embodiments, biocircuits of the present invention may be used as metabolite biosensors. As used herein a “metabolite biosensor” refers to biocircuits that are capable of interacting and responding to metabolites. Biosensors described herein may be used in vitro, in vivo, and/or ex vivo. Biosensors described herein may also be used in cells, tissues, or in entire organisms.

Biocircuits of the present invention may couple metabolite sensing to payloads comprising a reporter moiety. Examples of reporter moieties include but are not limited to fluorescence reporters such as green fluorescent protein (GFP), β-galactosidase, or bioluminescent reporters such as luciferase. Biosensors of the present invention are superior to conventional methods of detection since they are more sensitive and allow real-time monitoring of metabolite dynamics, especially labile and low abundant metabolites.

In some embodiments, biosensors of the present invention may be engineered to couple the sensing of a metabolite with payloads that confer a fitness advantage to the cells, tissues or organisms. Payloads may be biomolecules that are necessary for survival under selective conditions. Such methods are useful in enrichment of cells with certain desirable characteristics.

Metabolite biosensors consisting of biocircuits of the present invention may also be used to control metabolic flux dynamically. This may be achieved by using payloads that may tune pathway enzymes in response to the level of the relevant metabolite, allowing for dynamic control of pathway activity.

### Transgenic Organisms

In some embodiments, the present invention provides transgenic organisms that expresses nucleic acids that encode polypeptides of the present invention. As used herein the term “transgenic organism” refers to any non-human entity that contains artificially transferred, exogenous genetic material. This approach provides the ability to temporally regulate payloads within defined cells, tissues or in the entire organism. Such methods may be useful in creating transgenic models for certain disease states, or for studying embryonic development.

Biocircuits of the present invention may also be used to identify specific cell, tissues and/or organs within a transgenic organism where in a particular activity is located and effector modules described herein may designed to include a reporter moiety. Presence of the activity may result in the change in the expression of the reporter moiety.

In some embodiments, transgenic organisms may include biocircuits designed to be responsive a particular activity. Such activity may represent the stimulus. Effector modules may also include a cleavage site and/or a linker that is responsive to the activity within specific cells or tissues or organs within the organism. Cleavage at the cleavage site or linker releases the payload from the effector module.

Transgenic organisms described herein may include rodents, fish, reptiles, as well as invertebrates. In a preferred embodiment, such transgenic organisms may be selected from the rodent family including mouse, and rat.

### Gene Transcription

Protein levels and activities in a biological system are tightly regulated through many mechanisms. One important mechanism is transcriptional regulation which requires transcription factors to bind to specific DNA sequences in order to regulate the expression of a gene of interest. In some embodiments, pharmaceutical compositions, biocircuits, biocircuit components, effector modules including their SREs or payloads of the present invention may be used to tune transcription factors. The double tunable systems in which SREs (e.g. DDs) drive the expression of transcription factors and the transcription factors control a target gene may provide greater control and production of proteins in a cell, a tissue, an organ and/or a biological system.

### Tunable Regulations

The biocircuits of the present invention and/or any of their components may also be utilized to regulate the expression of another effector module such as a recombinant construct comprising a POI. In some embodiments, the biocircuits and/or effector modules may comprise a protease (also called peptidase or proteinase). The tunable protease could cleave an inactive construct to an active construct when the two components are co-introduced into a cell, a tissue or an organism.

In other examples, the biocircuits and/or effector modules comprising a protease may also be utilized to regulate protein processing including cleavage of the initial protein product to produce a smaller active protein or peptide.

In some embodiments, the biocircuits of the present invention and/or any of their components may comprise any of factors that play a role in protein processing and modification. Protein post-translational modification may include, but are not limited to, addition of hydrophobic groups by an enzyme (e.g., myristoylation, palmitoylation, isoprenylation, prenylation, farnesylation, geranylgeranylation, glypiation, and glycosylphosphatidylinositol (GPI) anchor); attachment of cofactors for enhanced function (e.g., lipoylation, flavin, phosphopantetheinylation, and heme C); addition of small chemical groups (e.g., acylation, formylation, alkylation, phosphorylation, methylation, arginylation, polyglutamylation, polyglycylation, butyrylation, glycosylation, propionylation, S-glutathionylation, S-nitrosylation, S-sulfenylation, succinylation, sulfation, and acetylation); linkage of othe proteins and/or peptides such as ISGylation, SUMOylation, ubiquitination, neddylation, and pupylation; chemical modification of amino acids; and structural changes.

### Boolean Switches

Biocircuits of the present inventions may also be incorporated into the design of cellular Boolean Switches. As used herein, a Boolean switch refers to a circuit that is designed to perform a logical operation based on one or more inputs and which produces an output. Logical operations performed by Boolean switches but are not limited to, AND, OR, NOR, NAND, NOT, IMPLY, NIMPLY, XOR, and XNOR. OR as well as AND gates represent the most fundament logical operations where OR represents a scenario where any of the one or more inputs is required to produce an output, while AND represents a scenario where all of the inputs are required to generate an output. Compound Boolean switches that consist of multiple logical operations may also be generated using biocircuits of the invention. In some embodiments, biocircuits and/or any of their components may represent one or more inputs in a Boolean switch. In other embodiments, biocircuits of the invention may be combined with switches known in the art to generate Boolean Switches. The output of a Boolean Switches may depend on the payload utilized. As a non limiting example, an AND based Boolean Switch may be generated where a first input comprises a biocircuit with gene editing nuclease, Cas9, as the payload and a second input comprises a biocircuit with transcriptional activator, VPR, as the payload. In the presence of the target gene guide RNA, addition of the stimuli to both inputs is required for the transcriptional activation of target gene (Gao Y et al. (2016) Nat Methods. 13(12):1043-1049; the contents of which are incorporated by reference in their entirety).

### Biofactories

The biocircuits of the present invention and/or any of their components may be utilized to regulate the levels of protein production in a biofactory. As used herein, the term “biofactory” refers to a cell, a tissue, an organ or an organism genetically modified or not, which can produce proteins with a number of applications including therapeutic purposes (inhibitors, enzymes, antibodies, antigens, etc.) or primary or secondary products of industrial interest. In some examples, the cell may be a prokaryotic cell, a eukaryotic cell, a mammalian cell, a plant cell, etc.

In some embodiments, the biocircuits of the present invention may be used to regulate medicament proteins produced in a target tissue, for example, the liver and the kidney. The liver is an organ that produces secreted proteins including major plasma proteins, factors in hemostasis and fibrinolysis, carrier proteins, hormones, prohormones and apolipoproteins, or a variety of short-lived metabolic peptides and enzymes which are usually tightly regulated, or other non-hepatic proteins. In the context, the liver fills a role of gene expression factory (biofactory), supplying a protein for treatment of a disease for example a metabolic disease.

In other embodiments, the biocircuits of the present invention may be used to regulate proteins for industrial processes.

### Liver Targeting

The liver is an important organ that produces proteins and involves blood clotting and a number of metabolic functions. A variety of diseases can affect liver and targeting the liver for disease treatment has been a promising approach, especially liver-targeted gene therapy. The biocircuits of the present invention and/or any of their components may be utilized to regulate liver targeted gene therapy and gene transfer.

Proteins that can be targeted to the liver and constructed to the present biocircuits for regulation may include those in liver cancers such as hepatocellular carcinoma (HCC), Fibrolamellar HCC, Cholangiocarcinoma, Angiosarcoma and secondary liver cancer; inherited disorders caused by defective genes such as hemochromatosis, Wilson disease, tyrosinemia, alpha 1 antitrypsin deficiency, glycogen storage disease; metabolic disorders due to enzyme deficiency such as Gilbert's syndrome, lysosomal acid lipase deficiency (LALD) and Gaucher disease; autoimmune hepatitis; fatty liver diseases; and viral hepatitis (A, B and C). In some examples, the present biocircuits may be used to direct IL-12 for hepatocellular carcinoma (HCC), and IL-10 for diabetic neuropathy.

In some embodiments, the present biocircuits may be used to control liver specific gene products for gene therapy.

In some embodiments, the present biocircuits may be used to control liver proteins that are secreted (e.g., to blood).

### Microfluidics

In some embodiments, cells containing biocircuits of the present invention and/or any of their components may be utilized in microfluidics devices. As used herein a “microfluidics device” refers to the manipulation of picoliter to nanoliter-scale volumes of fluids within artificially fabricated microsystems. Microfluidic devices comprising biocircuits of the present invention may be utilized to study cell culture models, cellular microenvironment, cell secretions, chemotaxis, apoptosis, vascular function, neuron cell growth, embryonic development, single cell metabolomics, gene expression, drug research, cellular separation, stem cell biology, bioreactors, three dimensional cell culture, and tissue engineering.

Microfluidics devices comprising biocircuits of the present invention may be used to express payload of interest within a subcellular region. In some embodiments, subcellular location specific expression of the payloads of interest may be achieved by delivery of the stimulus via channels within the microfluidic devices that generate a laminar flow near specific areas of the cell (Takayama S., et al. (2003). Chem Biol. 10(2):123-30; the contents of which are incorporated by reference in their entirety). In other embodiments, subcellular location specific expression of payloads of interest is achieved using Polydimethysiloxane-based culture devices as described in Uruyu D., et al., (2016) Sci Technol Adv Mater. 17(1):691-697 (the contents of which are incorporated by reference in their entirety).

### Tracking SREs, Biocircuits and Cell Lines

In some embodiments, it may be desirable to track the compositions of the invention or the cells modified by the compositions of the invention. Tracking may be achieved by using payloads such as reporter moieties, which, as used herein, refers to any protein capable of creating a detectable signal, in response to an input. Examples include alkaline phosphatase, β-galactosidase, chloramphenicol acetyltransferase, β-glucuronidase, peroxidase, β-lactamase, catalytic antibodies, bioluminescent proteins e.g. luciferase, and fluorescent proteins such as Green fluorescent protein (GFP).

Reporter moieties may be used to monitor the response of the SREs upon addition of the ligand corresponding to the SRE. In other instances, reporter moieties may be used to track cell survival, persistence, cell growth, and/or localization in vitro, in vivo, or ex vivo.

In some embodiments, the preferred reporter moiety may be luciferase proteins. In one embodiment, the reporter moiety is the Renilla luciferase, or a firefly luciferase. Table 8 provides the amino acid sequences and nucleic acid sequences of the reporter moieties.

TABLE 8
DD-luciferase constructs

| | | Amino |
|---|---|---|
| | | Acid |
| | | SEQ ID |
| Description | Amino acid sequence | NO. |
| Renilla | MTSKVYDPEQRKRMITGPQWWARCKQMNVLDSFINYYDSEKHA | 42 |
| luciferase | ENAVIFLHGNAASSYLWRHVVPHIEPVARCIIPDLIGMGKSGKSGN | |
| | GSYRLLDHYKYLTAWFELLNLPKKIIFVGHDWGACLAFHYSYEH | |
| | QDKIKAIVHAESVVDVIESWDEWPDIEEDIALIKSEEGEKMVLENN | |
| | FFVETMLPSKIMRKLEPEEFAAYLEPFKEKGEVRRPTLSWPREIPLV | |
| | KGGKPDVVQIVRNYNAYLRASDDLPKMFIESDPGFFSNAIVEGAK | |
| | KFPNTEFVKVKGLHFSQEDAPDEMGKYIKSFVERVLKNEQ | |
| Firefly | MEDAKNIKKGPAPFYPLEDGTAGEQLHKAMKRYALVPGTIAFTD | 43 |
| Luciferase | AHIEVDITYAEYFEMSVRLAEAMKRYGLNTNHRIVVCSENSLQFF | |
| | MPVLGALFIGVAVAPANDIYNERELLNSMGISQPTVVFVSKKGLQ | |
| | KILNVQKKLPIIQKIIIMDSKTDYQGFQSMYTFVTSHLPPGFNEYDF | |
| | VPESFDRDKTIALIMNSSGSTGLPKGVALPHRTACVRFSHARDPIF | |
| | GNQIIPDTAILSVVPFHHGFGMFTTLGYLICGFRVVLMYRFEEELFL | |
| | RSLQDYKIQSALLVPTLFSFFAKSTLIDKYDLSNLHEIASGGAPLSK | |
| | EVGEAVAKRFHLPGIRQGYGLTETTSAILITPEGDDKPGAVGKVVP | |
| | FFEAKVVDLDTGKTLGVNQRGELCVRGPMIMSGYVNNPEATNAL | |
| | IDKDGWLHSGDIAYWDEDEHFFIVDRLKSLIKYKGYQVAPAELESI | |
| | LLQHPNIFDAGVAGLPDDDAGELPAAVVVLEHGKTMTEKEIVDY | |
| | VASQVTTAKKLRGGVVFVDEVPKGLTGKLDARKIREILIKAKKGG | |
| | KSKL | |
| FKBP (F36V, | MGVQVETISPGDGRTFPKRGQTCVVHYTGMLEDGK | 44 |
| L106P) | KVDSSRDRNKPFKFMLGKQEVIRGWEEGVAQMSVG | |
| | QRAKLTISPDYAYGATGHPGIIPPHATLVFDVELLKP | |
| | E | |
| FKBP (E31G, | MGVQVETISPGDGRTFPKRGQTCVVHYTGMLEDGK | 45 |
| F36V, R71G, | KVDSSRDRNKPFKFMLGKQEVIRGWEEGVAQMSVG | |
| K105E) | QRAKLTISPDYAYGATGHPGIIPPHATLVFDVELLKP | |
| | E | |
| ecDHFR | MISLIAALAVDYVIGMENAMPWNLPADLAWFKRNTLNKPVIMGR | 46 |
| (R12Y, | HTWESIGRPLPGRKNIILSSQPGTDDRVTWVKSVDEAIAACGDVPE | |
| Y100I) | IMVIGGGRVIEQFLPKAQKLYLTHIDAEVEGDTHFPDYEPDDWES | |
| | VFSEFHDADAQNSHSYCFEILERR | |
| ecDHFR | ISLIAALAVDYVIGMENAMPWNLPADLAWFKRNTLNKPVIMGRH | 5 |
| (R12Y, | TWESIGRPLPGRKNIILSSQPGTDDRVTWVKSVDEAIAACGDVPEI | |
| Y100I) | MVIGGGRVIEQFLPKAQKLYLTHIDAEVEGDTHFPDYEPDDWESV | |
| | FSEFHDADAQNSHSYCFEILERR | |
| OT-Rluc-001 | MTSKVYDPEQRKRMITGPQWWARCKQMNVLDSFINYYDSEKHA | 47 |
| (Renilla Luc) | ENAVIFLHGNAASSYLWRHVVPHIEPVARCIIPDLIGMGKSGKSGN | |
| | GSYRLLDHYKYLTAWFELLNLPKKIIFVGHDWGACLAFHYSYEH | |
| | QDKIKAIVHAESVVDVIESWDEWPDIEEDIALIKSEEGEKMVLENN | |
| | FFVETMLPSKIMRKLEPEEFAAYLEPFKEKGEVRRPTLSWPREIPLV | |
| | KGGKPDVVQIVRNYNAYLRASDDLPKMFIESDPGFFSNAIVEGAK | |
| | KFPNTEFVKVKGLHFSQEDAPDEMGKYIKSFVERVLKNEQ | |
| OT-Fluc-002 | MGVQVETISPGDGRTFPKRGQTCVVHYTGMLEDGKKVDSSRDRN | 48 |
| (FKBP (F36V, | KPFKFMLGKQEVIRGWEEGVAQMSVGQRAKLTISPDYAYGATGH | |
| 106P); linker | PGIIPPHATLVFDVELLKPEEFMEDAKNIKKGPAPFYPLEDGTAGE | |
| (EF); Flue) | QLHKAMKRYALVPGTIAFTDAHIEVDITYAEYFEMSVRLAEAMK | |
| | RYGLNTNHRIVVCSENSLQFFMPVLGALFIGVAVAPANDIYNEREL | |
| | LNSMGISQPTVVFVSKKGLQKILNVQKKLPIIQKIIIMDSKTDYQGF | |
| | QSMYTFVTSHLPPGFNEYDFVPESFDRDKTIALIMNSSGSTGLPKG | |
| | VALPHRTACVRFSHARDPIFGNQIIPDTAILSVVPFHHGFGMFTTLG | |
| | YLICGFRVVLMYRFEEELFLRSLQDYKIQSALLVPTLFSFFAKSTLI | |
| | DKYDLSNLHEIASGGAPLSKEVGEAVAKRFHLPGIRQGYGLTETTS | |
| | AILITPEGDDKPGAVGKVVPFFEAKVVDLDTGKTLGVNQRGELCV | |
| | RGPMIMSGYVNNPEATNALIDKDGWLHSGDIAYWDEDEHFFIVD | |
| | RLKSLIKYKGYQVAPAELESILLQHPNIFDAGVAGLPDDDAGELPA | |
| | AVVVLEHGKTMTEKEIVDYVASQVTTAKKLRGGVVFVDEVPKGL | |
| | TGKLDARKIREILIKAKKGGKSKL | |
| OT-Rluc-003 | MGVQVETISPGDGRTFPKRGQTCVVHYTGMLEDGKKVDSSRDRN | 49 |
| (FKBP (F36V, | KPFKFMLGKQEVIRGWEEGVAQMSVGQRAKLTISPDYAYGATGH | |
| 106P); linker | PGIIPPHATLVFDVELLKPESGTSKVYDPEQRKRMITGPQWWARC | |
| (SG); Renilla | KQMNVLDSFINYYDSEKHAENAVIFLHGNAASSYLWRHVVPHIEP | |
| Luc | VARCIIPDLIGMGKSGKSGNGSYRLLDHYKYLTAWFELLNLPKKII | |
| | FVGHDWGACLAFHYSYEHQDKIKAIVHAESVVDVIESWDEWPDI | |
| | EEDIALIKSEEGEKMVLENNFFVETMLPSKIMRKLEPEEFAAYLEPF | |
| | KEKGEVRRPTLSWPREIPLVKGGKPDVVQIVRNYNAYLRASDDLP | |
| | KMFIESDPGFFSNAIVEGAKKFPNTEFVKVKGLHFSQEDAPDEMG | |
| | KYIKSFVERVLKNEQ | |
| OT-Rluc-004 | MISLIAALAVDYVIGMENAMPWNLPADLAWFKRNTLNKPVIMGR | 50 |
| (ecDHFR | HTWESIGRPLPGRKNIILSSQPGTDDRVTWVKSVDEAIAACGDVPE | |
| (R12Y, | IMVIGGGRVIEQFLPKAQKLYLTHIDAEVEGDTHFPDYEPDDWES | |
| Y100I); linker | VFSEFHDADAQNSHSYCFEILERRSGTSKVYDPEQRKRMITGPQW | |
| (SG); Renilla | WARCKQMNVLDSFINYYDSEKHAENAVIFLHGNAASSYLWRHV | |
| Luc) | VPHIEPVARCIIPDLIGMGKSGKSGNGSYRLLDHYKYLTAWFELLN | |
| | LPKKIIFVGHDWGACLAFHYSYEHQDKIKAIVHAESVVDVIESWD | |
| | EWPDIEEDIALIKSEEGEKMVLENNFFVETMLPSKIMRKLEPEEFA | |
| | AYLEPFKEKGEVRRPTLSWPREIPLVKGGKPDVVQIVRNYNAYLR | |
| | ASDDLPKMFIESDPGFFSNAIVEGAKKFPNTEFVKVKGLHFSQEDA | |
| | PDEMGKYIKSFVERVLKNEQ | |
| OT-Rluc-005 | MTSKVYDPEQRKRMITGPQWWARCKQMNVLDSFINYYDSEKHA | 51 |
| (Renilla Luc; | ENAVIFLHGNAASSYLWRHVVPHIEPVARCIIPDLIGMGKSGKSGN | |
| linker (SG); | GSYRLLDHYKYLTAWFELLNLPKKIIFVGHDWGACLAFHYSYEH | |
| FKBP (E31G, | QDKIKAIVHAESVVDVIESWDEWPDIEEDIALIKSEEGEKMVLENN | |
| F36V, R71G, | FFVETMLPSKIMRKLEPEEFAAYLEPFKEKGEVRRPTLSWPREIPLV | |
| K105E)) | KGGKPDVVQIVRNYNAYLRASDDLPKMFIESDPGFFSNAIVEGAK | |
| | KFPNTEFVKVKGLHFSQEDAPDEMGKYIKSFVERVLKNEQSGGVQ | |
| | VETISPGDGRTFPKRGQTCVVHYTGMLGDGKKVDSSRDRNKPFKF | |
| | MLGKQEVIRGWEEGVAQMSVGQGAKLTISPDYAYGATGHPGIIPP | |
| | HATLVFDVELLELE | |
| OT-Rluc-006 | MTSKVYDPEQRKRMITGPQWWARCKQMNVLDSFINYYDSEKHA | 52 |
| (Renilla Luc; | ENAVIFLHGNAASSYLWRHVVPHIEPVARCIIPDLIGMGKSGKSGN | |
| linker (SG); | GSYRLLDHYKYLTAWFELLNLPKKIIFVGHDWGACLAFHYSYEH | |
| ecDHFR | QDKIKAIVHAESVVDVIESWDEWPDIEEDIALIKSEEGEKMVLENN | |
| (R12Y, | FFVETMLPSKIMRKLEPEEFAAYLEPFKEKGEVRRPTLSWPREIPLV | |
| Y100I)) | KGGKPDVVQIVRNYNAYLRASDDLPKMFIESDPGFFSNAIVEGAK | |
| | KFPNTEFVKVKGLHFSQEDAPDEMGKYIKSFVERVLKNEQSGISLI | |
| | AALAVDYVIGMENAMPWNLPADLAWFKRNTLNKPVIMGRHTWE | |
| | SIGRPLPGRKNIILSSQPGTDDRVTWVKSVDEAIAACGDVPEIMVIG | |
| | GGRVIEQFLPKAQKLYLTHIDAEVEGDTHFPDYEPDDWESVFSEF | |
| | HDADAQNSHSYCFEILERR | |

### Chaperones

In some embodiments, effector modules of the present invention may include one or more chaperones to regulate the expression of the payload. Chaperones useful in the present invention may be cellular chaperones or small molecules referred to as pharmacological chaperones. Cellular chaperones refer to a large group of unrelated protein families whose role is to stabilize unfolded client proteins, or to unfold client proteins for translocation across membranes or for degradation, and/or to assist in their correct folding and assembly. Chaperones also cooperate with other components of the proteostasis network such as the proteasome system and autophagy to promote protein clearance. Examples of molecular chaperone families include small heat shock proteins such as hsp25; Heat shock protein 60 family proteins such as cpn60 and GroEL; Heat shock protein 70 family proteins such as DnaK and BiP; Heat shock protein 90 family proteins; Heat shock protein 100 family proteins such as CIp; lectin chaperones such as calnexin and calreticulin; and folding chaperones such as Protein disulfide isomerases (PDI), peptidyl prolyl ci-trans isomerase (PPI) and ERp57. In some embodiments, the payload of the present invention may be a cellular chaperone. In the absence of a stimulus which stabilizes the SRE, the cellular chaperone may bind to the SRE and is therefore unavailable to interact with its client proteins. In the presence of the stimulus specific to the SRE, the SRE is stabilized and the chaperone is available to interact with client proteins. In some embodiments, payloads of the present inventions may be appended to chaperones such that the stability or instability of the payload may be enhanced. In other embodiments, the SREs of the present invention may consist of one or more molecular chaperones.

Chaperones useful in the present invention may also include pharmacological chaperones which utilizes small molecules to facilitate the correct folding and stabilization of cellular proteins. Mutations in cellular proteins can result in protein misfolding and/or aggregation which ultimately results in their degradation. Pharmacological chaperones have been designed to bind to misfolded target proteins, facilitate their correct folding and thereby prevent their degradation. In some embodiments, SREs of the present invention may comprise one or more misfolded proteins and the stimulus specific to the SRE may include one or pharmacological chaperones such that the effector module is stabilized only in the presence of the pharmacological chaperone. In one example, the stimulus of the present invention may be pharmacological chaperone, deoxygalactonojirimycin (DGJ), and the SRE of present invention may be a R301Q mutant alpha Galacatose enzyme (see U.S. Pat. Nos. 6,274,597, 6,583,158, 6,589,964, 6,599,919, and 6,916,829; the contents of each of which are incorporated by reference in their entirety). In one embodiment, the SRE of the present invention may be acid β-glucosidase which may be stabilized by the isofagomine (IFG), and its derivatives, described in U.S. Pat. No. 6,583,158; the contents of which are incorporated by reference in their entirety.

T Cell Exhaustion

In some embodiments, biocircuits, their components, SREs or effector modules may be utilized to prevent T cell exhaustion. As used herein, “T cell exhaustion” refers to the stepwise and progressive loss of T cell function caused by chronic T cell activation. T cell exhaustion is a major factor limiting the efficacy of antiviral and antitumor immunotherapies. Exhausted T cells have low proliferative and cytokine producing capabilities concurrent with high rates of apoptosis and high surface expression of multiple inhibitory receptors. T cell activation leading to exhaustion may occur either in the presence or absence of the antigen.

In some embodiments, the biocircuits, and their components may be utilized to prevent T cell exhaustion in the context of Chimeric Antigen Receptor-T cell therapy (CAR-T). In this context, exhaustion in some instances, may be caused by the oligomerization of the scFvs of the CAR on the cell surface which leads to continuous activation of the intracellular domains of the CAR. As a non-limiting example, CARs of the present invention may include scFvs that are unable to oligomerize. As another non-limiting example, CARs that are rapidly internalized and re-expressed following antigen exposure may also be selected to prevent chronic scFv oligomerization on cell surface. In one embodiment, the framework region of the scFvs may be modified to prevent constitutive CAR signaling (Long et al. 2014. Cancer Research. 74(19) S1; the contents of which are incorporated by reference in their entirety). Tunable biocircuit systems of the present invention may be also used to regulate the surface expression of the CAR on the T cell surface to prevent chronic T cell activation. The CARs of the invention may also be engineered to minimize exhaustion. As a non-limiting example, the 41-BB signaling domain may be incorporated into CAR design to ameliorate T cell exhaustion. In some embodiments, any of the strategies disclosed by Long H A et al. may be utilized to prevent exhaustion (Long A H et al. (2015) Nature Medicine 21, 581-590; the contents of which are incorporated herein by reference in their entirety). In some embodiments, T cell metabolic pathways may be modified to diminish the susceptibility of T cells to exhaustion. Metabolic pathways may include, but are not limited to glycolysis, urea cycle, citric acid cycle, beta oxidation, fatty acid biosynthesis, pentose phosphate pathway, nucleotide biosynthesis, and glycogen metabolic pathways. As a non-limiting example, payloads that reduce the rate of glycolysis may be utilized to restrict or prevent T cell exhaustion (Long et al. Journal for Immunotherapy of Cancer 2013, 1(Suppl 1): P21; the contents of which are incorporated by reference in their entirety). In one embodiment, T cells of the present invention may be used in combination with inhibitors of glycolysis such as 2-deoxyglucose, and rapamycin.

In some embodiments, effector modules of the present invention, useful for immunotherapy may be placed under the transcriptional control of the T cell receptor alpha locus constant (TRAC) locus in the T cells. Eyquem et al. have shown that expression of the CAR from the TRAC locus prevents T cell exhaustion and the accelerated differentiation of T cells caused by excessive T cell activation (Eyquem J. et al (2017) Nature. 543(7643):113-117; the contents of which are incorporated herein by reference in their entirety).

In some embodiments, payloads of the invention may include, antibodies or fragments that target T cell surface markers associated with T cell exhaustion. T-cell surface markers associated with T cell exhaustion that may be used as payloads include, but are not limited to, CTLA-1, PD-1, TGIT, LAG-3, 2B4, BTLA, TIM3, VISTA, and CD96.

In one embodiment, the payload of the invention may be a CD276 CAR (with CD28, 4-IBB, and CD3 zeta intracellular domains), that does not show an upregulation of the markers associated with early T cell exhaustion (see International patent publication No. WO2017044699; the contents of which are incorporated by reference in their entirety).

### VIII. Definitions

Described herein are compositions, methods, processes, kits and devices for the design, preparation, manufacture and/or formulation of biocircuit systems.

At various places in the present specification, features or functions of the compositions of the present disclosure are disclosed in groups or in ranges. It is specifically intended that the present disclosure include each and every individual subcombination of the members of such groups and ranges. The following is a non-limiting list of term definitions.

Activity: As used herein, the term “activity” refers to the condition in which things are happening or being done. Compositions of the invention may have activity and this activity may involve one or more biological events. In some embodiments, biological events may include cell signaling events. In some embodiments, biological events may include cell signaling events associated protein interactions with one or more corresponding proteins, receptors, small molecules or any of the biocircuit components described herein.

Administered in combination: As used herein, the term “administered in combination” or “combined administration” refers to simultaneous exposure of one or more subjects to two or more agents administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigens of interest or desired antigens: As used herein, the terms “antigens of interest” or “desired antigens” refers to those proteins and/or other biomolecules provided herein that are immunospecifically bound or interact with antibodies of the present invention and/or fragments, mutants, variants, and/or alterations thereof described herein. In some embodiments, antigens of interest may comprise any of the polypeptides or payloads or proteins described herein, or fragments or portions thereof.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a compounds and/or compositions of the present invention may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules.

Candidate antibody: As used herein, the term “candidate antibody” refers to an antibody from a pool of one or more antibody from which one or more desired antibodies may be selected.

Cellular matrix: As used herein, the term “cellular matrix” refers to the biochemical and structural environment associated with the outer portion of the cell membrane. Such cell membranes may also include platelet membranes. Components of the cellular matrix may include, but are not limited to proteoglycans, carbohydrate molecules, integral membrane proteins, glycolipids and the like.

Compound: As used herein, the term “compound,” refers to a distinct chemical entity. The term may be used herein to refer to peptides, proteins, protein complexes, nucleic acids, polynucleotides or antibodies of the invention. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

CRISPR-Cas system: As used herein, the term “CRISPR-Cas system” in general refers collectively to components/elements involved in directing the activity of CRISPR-associated (“Cas”) proteins, including sequences encoding a Cas gene, a tracr (trans-activating CRISPR) sequence (e.g. tracrRNA or an active partial tracrRNA), a tracr-complementary sequence (encompassing a “direct repeat” and a tracrRNA-processed partial direct repeat in the context of an endogenous CRISPR system), a target (guide) sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR-Cas system. In some embodiments, one or more elements of a CRISPR-Cas system is derived from a particular organism which may comprise an endogenous CRISPR-Cas system, such as Streptococcus pyogenes. In general, a CRISPR-Cas system is characterized by components that promote the formation of a CRISPR/Cas complex at the site of a target sequence (also referred to as a protospacer in the context of an endogenous CRISPR-Cas system). In the context of formation of a CRISPR complex, “target sequence” refers to a sequence to which a target recognition sequence is designed to have complementarity, where hybridization between a target sequence and a target recognition sequence promotes the formation of a CRISPR/Cas complex. A target sequence may comprise any polynucleotide, such as DNA or RNA polynucleotides. In some embodiments, a target sequence is located in the nucleus or cytoplasm of a cell.

CRISPR interference (CRISPRi): As used herein, the term “CRISPRi” refers to a genetic perturbation technique that allows for sequence-specific repression or activation of gene expression in prokaryotic and eukaryotic cells using CRISPR complexes. CRISPRi regulates gene expression primarily on the transcriptional level.

Delivery: As used herein, “delivery” refers to the act or manner of delivering a compound, substance, entity, moiety, cargo or payload.

Delivery Agent: As used herein, “delivery agent” refers to any agent which facilitates, at least in part, the in vivo delivery of one or more substances (including, but not limited to compounds and/or compositions of the present invention) to a cell, subject or other biological system cells.

Desired antibody: As used herein, the term “desired antibody” refers to an antibody that is sought after, in some cases from a pool of candidate antibodies.

Destabilized: As used herein, the term “destable,” “destabilize,” “destabilizing region”, or “destabilizing domain” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Distal: As used herein, the term “distal” means situated away from the center or away from a point or region of interest.

Engineered: As used herein, embodiments of the invention are “engineered” when they are designed to have a feature or property, whether structural or chemical, that varies from a starting point, wild type or native molecule.

Epitope: As used herein, an “epitope” refers to a surface or region on a molecule that is capable of interacting with components of another molecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Extracellular matrix: As used herein, the term, “extracellular matrix,” or “ECM” refers to the area surrounding cells and/or the area between cells that typically comprises structural proteins as well as cell signaling molecules. Components of the extracellular matrix may include, but are not limited to proteins, nucleic acids, membranes, lipids and sugars that may be directly or indirectly associated with structural components of the extracellular environments. Structural components of the extracellular matrix may include, but are not limited to proteins, polysaccharides (e.g. hyaluronic acid) glycosaminoglycans and proteoglycans (e.g. heparin sulfate, chondroitin sulfate and keratin sulfate.) Such structural components may include, but are not limited to fibrous components (e.g. collagens and elastins) fibrillins, fibronectin, laminins, agrin, perlecan, decorin and the like.

Ex Vivo: As used herein, the term “ex vivo” refers to events that occur outside an organism (e.g., animal, plant, or microbe or cell or tissue thereof).

Feature: As used herein, a “feature” refers to a characteristic, a property, or a distinctive element.

Formulation: As used herein, a “formulation” includes at least a compound and/or composition of the present invention and a delivery agent.

Fragment: A “fragment,” as used herein, refers to a portion. For example, fragments of proteins may comprise polypeptides obtained by digesting full-length protein. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody.

Functional: As used herein, a “functional” biological molecule is a biological entity with a structure and in a form in which it exhibits a property and/or activity by which it is characterized.

Guide RNA (gRNA): As used herein, the term “guide RNA” refers to a RNA molecule used in conjunction with a CRISPR associated system. The guide RNA may be composed of two RNA molecules, i.e., one RNA (“crRNA”) which hybridizes to a target sequence and provides sequence specificity, and one RNA, the “tracrRNA”, which is capable of hybridizing to the crRNA and forming a duplex with crRNA upon hybridization. In some embodiments the guide RNA may be a single guide RNA (sgRNA). sgRNA contains nucleotide sequence specific to a non-variable scaffold sequence of the 5′ end of a target DNA (i.e. crRNA) and tracrRNA sequence. SgRNA can be delivered as RNA or by transforming with a plasmid with sgRNA coding sequence under a promotor sequence. The base pairing of sgRNA with the target sequence recruits a Cas protein (e.g., the Cas9 nuclease) to bind the DNA at that locus and cleave the target DNA sequence. As used herein, the term “crRNA” is intended to refer to the endogenous bacterial RNA that confers target specificity, which requires tracrRNA to bind to Cas9. As used herein, the term “tracrRNA” refers to the endogenous bacterial RNA that links the crRNA to the Cas9 nuclease and can bind any crRNA.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically, a reduction in the level of mRNA results in a reduction in the level of a polypeptide translated therefrom. The level of expression may be determined using standard techniques for measuring mRNA or protein.

In situ: As used herein, the term “in situ” refers to events that occur in the original, natural, or existing environment e.g. within an organism.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Linker: As used herein, a linker refers to a moiety that connects two or more domains, moieties or entities. In one embodiment, a linker may comprise 10 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, a linker may comprise one or more nucleic acids comprising one or more nucleotides. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent). or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl)phosphine (TCEP), or other reducing agents, and/or photolysis, as well as an ester bond which may be cleaved for example by acidic or basic hydrolysis.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” means existing in nature without artificial aid, or involvement of the hand of man.

Niche: As used herein, the term “niche” refers to a place, zone and/or habitat. In some embodiments, niches comprise cellular niches. As used herein, the term “cell niche” refers to a unique set of physiologic conditions in a cellular system within a tissue, organ or organ system within or derived from a mammalian organism. A cell niche may occur in vivo, in vitro, ex vivo, or in situ. Given the complex nature and the dynamic processes involved in growth factor signaling, a cell niche may be characterized functionally, spatially or temporally or may be used to refer to any environment that encompasses one or more cells. As such, in some embodiments a cell niche includes the environment of any cell adjacent to another cell that provides support, such as for example a nurse cell.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene and/or cellular transcript.

Operably linked: As used herein, the phrase “operably linked” refers to a functional connection between two or more molecules, constructs, transcripts, entities, moieties or the like.

Paratope: As used herein, a “paratope” refers to the antigen-binding site of an antibody.

Passive adsorption: As used herein, “passive adsorption” refers to a method of immobilizing solid-phase reactants on one or more surfaces (e.g. membranes, dishes, culture dishes, assay plates, etc.) Immobilization typically occurs due to affinity between such reactants and surface components.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N, N′-dimethylformamide (DMF), N, N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may be covalently bonded or sequestered in some way until converted into the active drug moiety prior to, upon or after administration to a mammalian subject. Prodrugs can be prepared by modifying functional groups present in the compounds in such a way that the modifications are cleaved, either in routine manipulation or in vivo, to the parent compounds. Prodrugs include, among other things, compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety. The effector modules of the present invention may act as or be considered prodrugs.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acids to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, that include the C-terminus, but do not comprise the N-terminus.

Region of antibody recognition: As used herein, the term “region of antibody recognition” refers to one or more regions on one or more antigens or between two or more antigens that are specifically recognized and bound by corresponding antibodies. In some embodiments, regions of antibody recognition may comprise 1, 2, 3, 4, 5, 6, 7, 8, 9 or at least 10 amino acid residues. In some embodiments, regions of antibody recognition comprise a junction between two proteins or between two domains of the same protein that are in close proximity to one another.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a secondary status or state or to a reference compound or entity.

Stimulus response element (SRE): the term “stimulus response element (SRE), as used herein, is a component of an effector module which is joined, attached, linked to or associated with one or more payloads of the effector module and in some instances is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1 and 100 or a factored increase or decrease such as 2-fold, 3-fold, 4-fold, 5-fold, 10-fold or more. One non-limiting example of an SRE is a destabilizing domain (DD).

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, ex vivo, in situ or in the tissue or organ of an organism. The organism may be an animal, preferably a mammal, more preferably a human and most preferably a patient.

Target site: The term “target site” as used herein, refers to a region or area targeted by a given compound, composition or method of the invention. Target sites may include, but are not limited to cells, tissues, organs, organ systems, niches and the like.

Therapeutic Agent: The term “therapeutic agent” refers to any agent that, when administered to a subject, has a therapeutic, diagnostic, and/or prophylactic effect and/or elicits a desired biological and/or pharmacological effect. Therapeutic agents of the present invention include any of the biocircuit components taught herein either alone or in combination with other therapeutic agents.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in a 24 hr period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Tune: As used herein, the term “tune” means to adjust, balance or adapt one thing in response to a stimulus or toward a particular outcome. In one non-limiting example, the SREs and/or DDs of the present invention adjust, balance or adapt the function or structure of compositions to which they are appended, attached or associated with in response to particular stimuli and/or environments.

Unmodified: As used herein, “unmodified” refers to any substance, compound or molecule prior to being changed in any way. Unmodified may, but does not always, refer to the wild type or native form of a biomolecule or entity. Molecules or entities may undergo a series of modifications whereby each modified product may serve as the “unmodified” starting or reference molecule or entity for a subsequent modification.

### EXAMPLES

### Example 1. Screening Method to Identify Ligand Responsive SREs or DDs

### Study Design

To engineer constructs that display ligand dependent stability, a candidate ligand binding domain (LBD) is selected and a cell-based screen using yellow fluorescent protein (YFP) as a reporter for protein stability is designed to identify mutants of the candidate LBD possessing the desired characteristics of a destabilizing domain: low protein levels in the absence of a ligand of the LBD, (i.e., low basal stability), large dynamic range, robust and predictable dose-response behavior, and rapid kinetics of degradation (Banaszynski, et al., (2006) Cell; 126(5): 995-1004). The candidate LBD binds to a desired ligand but not endogenous signaling molecules.

The candidate LBD sequence (as a template) is first mutated using a combination of nucleotide analog mutagenesis and error-prone PCR, to generate libraries of mutants based on the template candidate domain sequence. The libraries generated are cloned in-frame at either the 5′- or 3′-ends of the YFP gene, and a retroviral expression system is used to stably transduce the libraries of YFP fusions into NIH3T3 fibroblasts.

### Screening Strategy I

The transduced NIH3T3 cells are subjected to three to four rounds of sorting using fluorescence-activated cell sorting (FACS) to screen the libraries of candidate DDs. Transduced NIH3T3 cells are cultured in the absence of the high affinity ligand of the ligand binding domain (LBD), and cells that exhibit low levels of YFP expression are selected through FACS. The selected cell population is cultured in the presence of the high affinity ligand of the ligand binding domain for a period of time (e.g., 24 hours), at which point cells are sorted again by FACS. Cells that exhibit high levels of YFP expression are selected through FACS and the selected cell population is split into two groups and treated again with the high affinity ligand of the ligand binding domain at different concentrations; one group treated with the lower concentration of the ligand and the other treated with a high concentration of the ligand, for a period of time (e.g., 24 hours), at which point cells are sorted again by FACS. Cells expressing mutants that are responsive to lower concentrations of the ligand are isolated.

The isolated cells responsible to the lower concentration of the ligand are treated with the ligand again and cells exhibiting low fluorescence levels are collected 4 hours following removal of the ligand from the media. This fourth sorting is designed to enrich cells that exhibit fast kinetics of degradation (Iwamoto et al., Chem Biol. 2010 Sep. 24; 17(9): 981-988).

The collected cells after four rounds of sorting are recovered. The identified candidate cells are harvested and the genomic DNA is extracted. The candidate DDs are amplified by PCR and isolated. The candidate DDs are sequenced and compared to the LBD template to identify the actual mutations in candidate DDs.

### Screening Strategy II

The selected cell population is subject to additional one or more sorts by FACS in the absence of high affinity ligand of LBD and cells that exhibit low levels of YFP expression are selected for further analysis. Cells are treated with high affinity ligand of the ligand binding domain, for a period of time (e.g. 24 hours), and sorted again by FACS. Cells expressing high levels of YFP are selected for through FACS. Cells with high expression of YFP are treated with ligand again and cells exhibiting low fluorescence levels are collected 4 hours following removal of the ligand from the media to enrich cells that exhibit fast kinetics of degradation. Any of the sorting steps may be repeated to identify DDs with ligand dependent stability.

### Example 2. E. coli DHFR (ecDHFR)-TMP Driven Assays

The ecDHFR plus trimethoprim system may be used to screen and or identify constructs useful in the invention. It was first described in 2010 (Iwamoto et al., Chem Biol. 2010; 17(9):981-8, the contents of which are incorporated herein by reference in their entirety).

Briefly, using this system, mutants of E. coli dihydrofolate reductase (ecDHFR) protein are engineered to be degraded when expressed in mammalian cells. When a destabilizing domain (DD) is fused to a protein of interest, its instability is conferred to the fused protein. Trimethoprim (TMP) is a high-affinity ligand for ecDHFR that stabilizes fusion proteins in a dose-dependent manner. The ability of TMP to cross the blood-brain barrier (BBB) enables the tunable regulation of proteins expressed in cells found within the mammalian central nervous system. The ecDHFR-TMP DD system can work in parallel to the existing FKBP/Shld1-based DD system, allowing simultaneous regulation of two proteins independently.

### Example 3. Regulation of Protein Expression in Mammalian Cells and/or Systems

### Regulation of Protein Expression in Mammalian Cells

Standard molecular cloning techniques are used to generate a fusion of the signal response element (SRE), e.g., a destabilization domain (DD) and the payload of interest (POI) into a retroviral plasmid, generating a SRE-POI construct. An epitope tag, FLAG-tag is appended to the protein of interest. The plasmids are packaged into viral particles following standard viral infection protocol. Harvested viral particles proceed immediately to creation of cell lines to test regulation of the POI expression in response to the ligand; or are frozen at −80° C. for future use.

To create a mammalian cell line containing the integrated SRE-POI transgene, NIH3T3 cells are plated and 3 mL filtered viral supernatant is added to the culture media. After four hours of infection, the viral media is replaced with 3T3 culture media. 48 hours after infection, cells with stable integration of the SRE-POI construct are selected by FACS and a fluorescent marker.

To test for ligand-dependent control of protein stability, equal numbers of cells with stable integration of the SRE-POI construct are plated in two cell culture plates; a high affinity ligand of the SRE is added in one plate and the other one is added with an equal volume of ethanol as a control. After incubation for 4 to 24 hours, the POI stability is assayed using either flow cytometry or western blotting.

A dose-response experiment using varying concentrations of the ligand and a time course assay is performed to test the ligand-dependent protein expression levels.

### Regulation of Protein Expression in Living Animals (Mice)

The SRE-POI construct and cells with stable integration of the SRE-POI construct are created following the same procedure as discussed above. A tumor xenograft is used as a mechanism to delivery transgene in mice (Banaszynski et al., Nat. Med., 2008, 14:1123-1127).

Day 1: Culture cells containing a stably integrated SRE-POI transgene to 80% confluence and count number of cells. Adjust the number of cells to implant per animal depending on the growth of the cell line in animals.

Day 2: Trypsinize, quench with complete media, and spin cells. Wash cells three times with PBS, and resuspend cells in 100 μL (10,000 cells per μL) of DMEM (no FBS) per animal. Xenograft cells subcutaneously (or at desired location) into mice anesthetized with isoflurane (2%).

After transplanted cells form stable grafts, treatment of the ligand of the SRE begins. The ligand is reconstituted in an injectable solution (e.g., 9:1 PEG400: Tween 80) at various concentrations, and injected intravenously to a test animal. A control animal is injected with the injection vehicle only.

Day 1 after the injection: The expression level of the POI is analyzed for testing experimental SRE-POI stability. For direct protein measurement, tumors are removed and the tumor tissue amount is standardized for tumor samples from each test animal. Tumor tissues are homogenized and assayed for protein levels via ELISA or immunoblotting using antibodies specific to the POI.

Mice are dosed with the ligand every 48 hours. SRE-POI stabilization is periodically analyzed for the phenotypic and/or functional effects of protein stabilization. For example, the tumor xenograft regression/size is measured as functional effects of POI stabilization dependent on the ligand treatment.

### Example 4. DD Regulated IL2 Expression

FKBP (L106P) and ecDHFR (R12Y, Y100I) are well-characterized destabilizing domains which can confer instability to fusion partners (e.g., a POI). The instability is reversed by a synthetic ligand named Shield-1 that binds to FKBP; TMP that binds to DHFR. An IL2 polypeptide was linked to either FKBP (L106P) or ecDHFR (R12Y, Y100I). IL2 constructs were cloned into pLVX-IRES-Puro lentiviral vectors. An IL2 signal sequence was inserted at the N terminus of the construct.

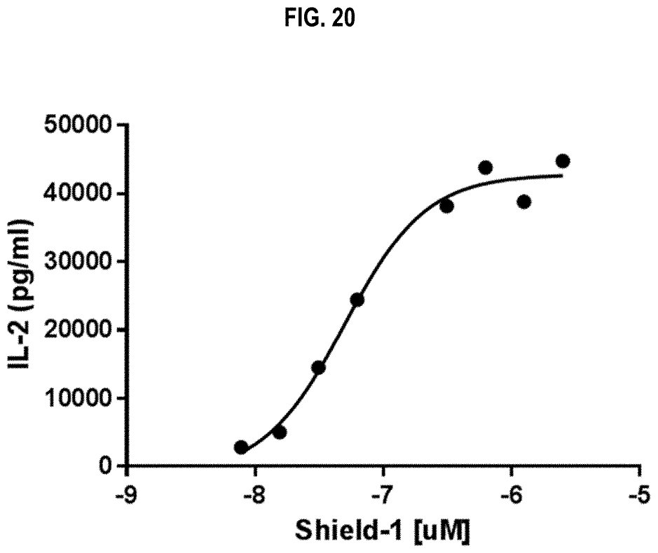

To evaluate dependence of IL2 levels on Shield-1 dose, HCT116 cells were plated onto a 96-well plate and treated with varying concentrations of Shield-1. Media was then collected from cells and IL2 levels were quantified using IL2 ELISA (FIG. 20). IL2 increased with increase in Shield-1 concentration and plateaued at higher Shield-1 dose levels. The EC50 of Shield-1 was determined to be 50 nM.

### Example 5. DD Regulated IL12 Expression

Several FKBP (DD)-IL12 and DHFR (DD)-IL12 constructs (as shown in Table 5) were cloned into pLVX-IRES-Puro lentiviral vectors. FKBP (DD) is positioned at either N-terminus (Construct ID #OT-IL12-001, OT-IL12-002 and OT-IL12-003) or C-terminus (OT-IL12-004, OT-IL12-005 and OT-IL12-009) of the fusion construct. ecDHFR (R12Y, Y100I) is located at the N-terminal end of the fusion construct (OT-IL12-007). A p40 signal sequence was inserted next to the DD or IL-12. In several constructs, a furin protease cleavage site or a modified furin site was included.

HEK293T cells were transiently transfected with 200 ng or 1 μg FKBP-IL12 plasmids (OT-IL12-001, OT-IL12-002, OT-IL12-003, OT-IL12-004, and OT-IL12-005), and treated with 10 μM Shield-1 or left untreated for 6 hours. Culture media was collected from transfected cells and diluted 1:50 to measure IL12 levels using p40 ELISA. Average IL12 ELISA readings are presented in Table 9.

TABLE 9
IL12 induction after transient transfection

| Construct ID | 10 μM Shield-1 | No Shield-1 |
|---|---|---|
| OT-IL12-001 | 1748.95 | 1289.61 |
| OT-IL12-002 | 50.73 | 18.01 |
| OT-IL12-003 | 2138.25 | 1762.55 |
| OT-IL12-004 | 1567.62 | 385.95 |
| OT-IL12-005 | 2670.80 | 1188.42 |
| HEK293T | −22.04 | −12.92 |

Treatment with Shield-1 resulted in a significant increase in IL12 over untreated with OT-IL12-004, and OT-IL12-005 constructs. OT-IL12-001, and OT-IL12-003 showed only modest increase in IL12 levels following Shield-1 treatment.

IL12 levels were also measured in cells following stable transfection. 500,000 cells stably transduced with OT-IL12-004 were plated in a 12 well plate and incubated overnight in growth media consisting of Dulbecco's Modified Eagle medium (DMEM) containing 10% fetal bovine serum (FBS). The next day the cells were treated with 1 μM Shield-1 or left untreated for 6 or 24 hours. Following treatment with Shield-1, growth media was collected from the cells and diluted 10, 40, 160 or 640 fold and FKBP-IL12 levels were quantified using IL12-p40 ELISA assay. Average IL12 ELISA readings are presented in Table 10.

TABLE 10
IL12 induction after stable transfection

| Media | 6 hours | | 24 hours | |
|---|---|---|---|---|
| dilution | 1 μM | No | 1 μM | No |
| (fold) | Shield-1 | Shield-1 | Shield-1 | Shield-1 |
| 10 | 0.58 | 0.17 | 1.33 | 0.28 |
| 40 | 0.26 | 0.10 | 0.79 | 0.12 |
| 160 | 0.12 | 0.08 | 0.31 | 0.09 |
| 640 | 0.08 | 0.09 | 0.12 | 0.08 |

IL12 induction following both 6 and 24 hours of Shield-1 treatment was most prominent at the lowest dilution factor of 10. A mild induction of IL12 induction was also observed at media dilution factor of 40.

To evaluate Shield-1 dependent FKBP-IL12 induction over time, 2 million cells were plated in growth medium and incubated overnight in the presence of 1 μM Shield-1 or left untreated. Cells were then incubated for an additional 2-72 hours and growth media was collected for the cells at all time points. Growth media was diluted 400 fold and IL12 levels were measured using IL12 p40 ELISA. Average IL12 ELISA readings are presented in Table 11.

TABLE 11
IL12 induction over time

| Time (hrs) | 1 μM Shield-1 | No Shield-1 |
|---|---|---|
| 2 | 0.1774 | 0.12615 |
| 4 | 0.2567 | 0.1359 |
| 6 | 0.29085 | 0.12655 |
| 8 | 0.2752 | 0.1385 |
| 24 | 0.99475 | 0.1819 |
| 48 | 1.78525 | 0.23145 |
| 72 | 1.6288 | 0.25955 |

IL12 Expression in Shield-1 Treated Cells was Higher than Untreated Following 24, 48 and 72 Hours after Shield-1 Treatment.

To evaluate the dependence of FKBP-IL12 production on Shield-1 dose levels, OT-IL12-004 transduced HEK293T cells were plated at different densities (40,000 cells, 20,000 cells, 10,000 cells or 5,000 cells per well) onto a 96-well plate. Following overnight incubation, cells were treated with growth medium containing 0 to 10 μM Shield-1 for 24 hours. Media was then collected, diluted 400 fold and FKBP-IL12 levels were measured using IL12-p40 ELISA. Average IL12 ELISA readings are presented in Table 12.

TABLE 12
Dose and cell number dependent IL12 induction

| Shield- | 40000 | 20000 | 10000 | 5000 |
|---|---|---|---|---|
| 1 (μM) | cells/well | cells/well | cells/well | cells/well |
| 10.00 | 623.77 | 656.70 | 214.11 | 193.62 |
| 3.33 | 670.64 | 618.10 | 273.74 | 207.55 |
| 1.11 | 677.27 | 872.24 | 322.56 | 203.71 |
| 0.37 | 368.17 | 582.71 | 250.49 | 172.50 |
| 0.12 | 197.29 | 343.34 | 156.98 | 95.92 |
| 0.04 | 171.50 | 205.68 | 63.79 | 48.89 |
| 0.01 | 117.25 | 103.56 | 13.30 | −2.35 |
| 0.00 | 66.34 | 60.58 | 2.11 | −8.53 |
| 0.00 | 100.43 | 39.55 | −13.58 | −21.76 |
| 0.00 | 83.49 | 7.92 | −21.76 | −26.97 |

A dose dependent IL12 induction was observed at all cell numbers tested. IL12 induction increased with Shield-1 up to a dose of 1 μM; following which IL12 induction plateaued. Notably, greater IL12 induction was observed at 2000 and 4000 cells/well.

### Example 6. FKBP and ecDHFR Regulated IL12 Mediated Functions

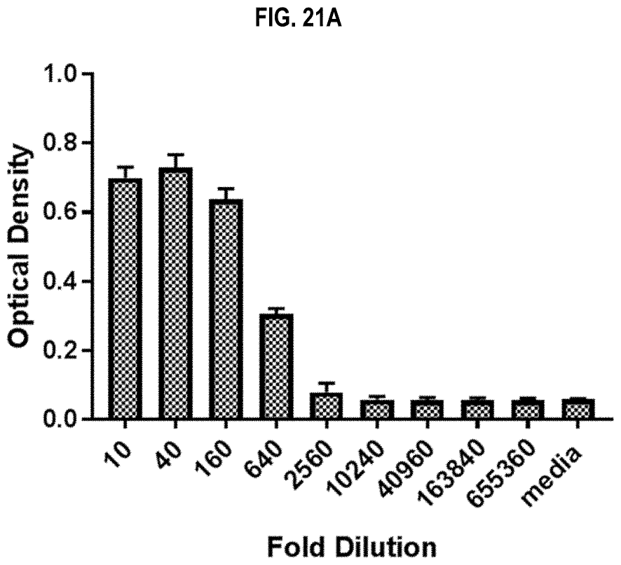

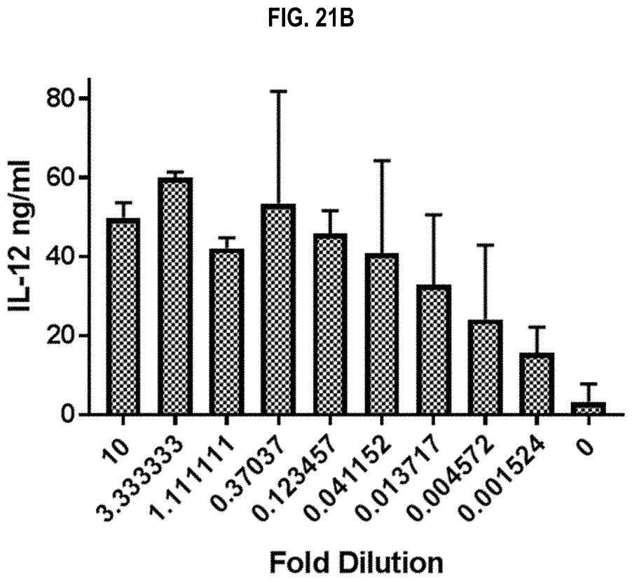

HEK-Blue sensor cells (InvivoGen, San Diego, Calif.) were utilized to evaluate whether DD regulated IL12 is capable of regulating signaling downstream of IL12. In these cells, the IL12 receptor, STAT4 and downstream transcriptional elements are tied to a reporter gene such that IL12 signaling can be monitored. To evaluate production of functional IL12, one million HEK 293T were transfected with 200 ng of OT-IL-12-003 plasmid using Lipofectamine 2000 (Thermo Fisher Scientific, Waltham, Mass.). 48 hours after transfection, cells were treated with growth media containing 10 μM Shield-1, incubated for another 24 hours, following which, media was collected. 50,000 HEK 293 Blue sensor cells were plated onto 96 well plates and incubated overnight with media (at different dilutions) from Shield-1 treated OT-IL12-003 expressing HEK293 T cells. After overnight incubation, 20 μl media was removed from each well and incubated with 180 μl Quanti-Blue reagent (InvivoGen, San Diego, Calif.) for 30 minutes at 37° C. Absorption was measured at 620 nm using a spectrophotometer. To generate a standard curve, 180 μl Quanti-Blue reagent was mixed with 20 μl□of recombinant IL12 at following concentrations 500, 250, 125, 62.5, 31.25, 15.62, 7.8 and 3.9 pg/ml. Functional IL12 concentrations were determined by comparing the optical density of each sample with IL12 standard curve. Measurable levels of functional IL12 were reached with 640 fold dilutions of IL12 containing growth media and further plateaued at higher concentrations of the media (FIG. 21A).

The dependence of functional IL12 production on the dose of Shield-1 used was also evaluated. 10,000 HEK293T cells stably transduced with OT-IL-12-004 were plated onto 96 well plated and treated with growth media containing 10, 3.33, 1.11, 0.37, 0.12, 0.04, 0.01, 0.005, 0.002 or 0 μM Shield-1 for 24 hours. Following Shield-1 treatment, media from cells was diluted 200 fold and 20 μL of the diluted media was added to HEK Blue sensor cells. After overnight incubation, 20 μl□of media was removed from each well and incubated with 180 μl Quanti-Blue reagent (InvivoGen, San Diego, Calif.) for 30 minutes at 37° C. Absorption was measured at 620 nm using a spectrophotometer. To generate a standard curve, 180 μl Quanti-Blue reagent was mixed with 20 μl□of recombinant IL12 at following concentrations 500, 250, 125, 62.5, 31.25, 15.62, 7.8 and 3.9 pg/ml. Functional IL12 concentrations were determined by comparing the optical density of each sample with IL12 standard curve. A dose dependent increase in the levels of functional IL12 levels was observed (FIG. 21B).

### Example 7. DD Regulated Luciferase

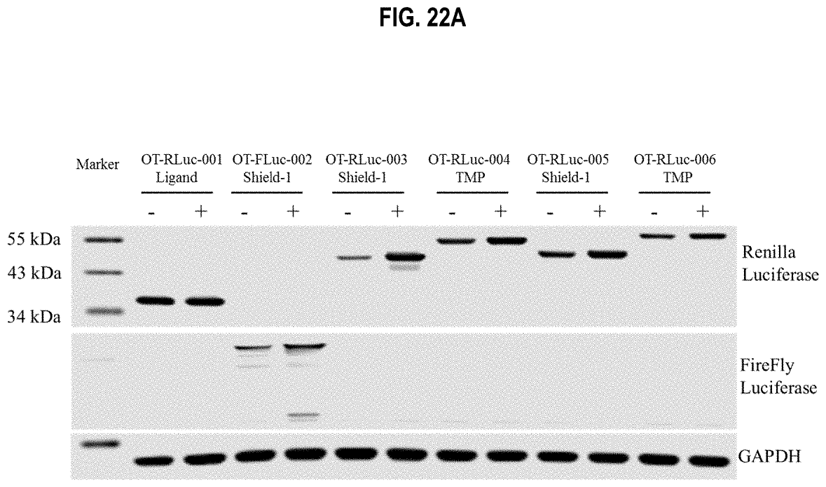

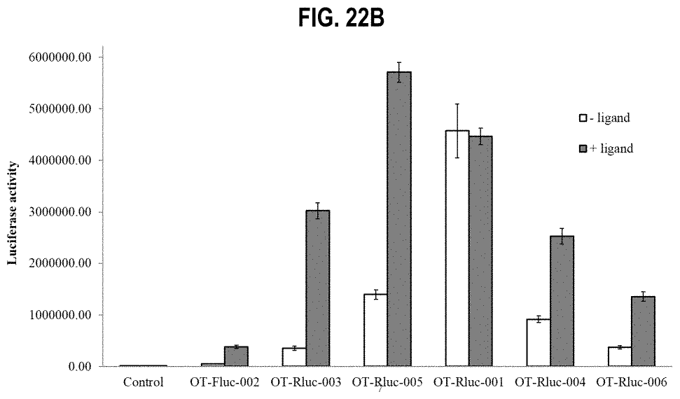

DD regulated luciferase can be used to track cells in vivo e.g. T cells. Firefly luciferase or Renilla luciferase may be utilized as the payload. HCT-116 cells were stably transduced with the constitutive (OT-RLuc-001) or DD regulated constructs (OT-RLuc-002, OT-RLuc-003, OT-RLuc-004, OT-RLuc-005 and OT-RLuc-006). Cells were treated with 1 μM Shield-1, or 10 μM Trimethoprim or vehicle control for 24 hours and luciferase expression and activity was measured. Luciferase expression was measured via western blotting using Anti-Renilla luciferase and anti-Firefly luciferase antibodies (Abcam, Cambridge, UK). Blots were also probed with anti-GAPDH antibody to ensure even protein loading in all samples. As expected, the constitutive luciferase construct (OT-RLuc-001) showed expression of Renilla luciferase both in the presence and absence of ligand. In contrast, OT-RLuc-003 showed strong Shield-1 dependent stabilization of Renilla luciferase. OT-RLuc-004, 005 and 006 showed modest stabilization of Renilla luciferase in the presence of their corresponding ligand, while OT-FLuc-002 showed modest stabilization of firefly luciferase with the addition of Shield-1 (FIG. 22A).

Ligand dependent activity of Renilla and firefly luciferase constructs was also measured in using coelentrazine and luciferin substrates respectively. Cells were treated with 1 μM Shield-1, or 10 μM Trimethoprim or vehicle control for 24 hours, lysed with assay lysis buffer and incubated with the luciferase substrate. Luciferase activity was measured as luminescence reading using a luminometer and the values were compared to control comprising of lysis buffer and substrate. In this assay, all DD regulated showed ligand dependent increase in luciferase activity compared to control. As expected, the constitutive construct OT-RLuc-001 showed high luciferase activity both in the presence and absence of ligand (FIG. 22B).

### Example 8. Regulation of Glucagon Expression in Mammalian Cells and/or Systems for the Treatment of Hypoglycemia

Hypoglycemia refers to a condition when the blood glucose levels are below normal. Blood glucose levels can be restored by administering glucagon. To generate biocircuits for the treatment of hypoglycemia, SREs fused to the payload of interest, glucagon, are cloned into retroviral vectors using standard molecular cloning techniques. Optional furin cleavage sites are included in the SREs to induce secretion of glucagon in non-secretory cells. NIH3T3 cells are transfected with retroviral vectors. Transfected cells are treated with high affinity ligand of the SRE and the glucagon expression is measured in the media using western blotting. Changes in glucose levels in the media is measured using mass spectrophotometry. An increase in glucagon protein levels and a concomitant increase in glucose levels in the media indicates that the ligand is able to stabilize the construct thereby allowing glucagon expression.

### Example 9. SRE-Based Cas9 Expression Systems

CRISPR/Cas9 technology has been widely used to generate heritable genomic changes. However, constitutive expression of Cas9 can result in toxicity and off-target effects. These limitations may be overcome by generating a regulatable Cas9 expression system where the Cas9 is fused to an SRE. Lentiviral vectors consisting of a first vector where a single guide RNA (sgRNA) targeting a gene of interest is driven by a constitutive promoter, and a second vector containing Cas9 fused at its N-terminal with a ligand-responsive SRE under the control of a suitable promoter are generated (see FIG. 19A and FIG. 19B). The constructs are then transduced into target cells such as cancer cells and embryonic stem cells. The expression levels of Cas9 and target gene of interest are measured in the presence or absence of varying doses of the ligand specific to the SRE, using western blot and RT-PCR. Cas9 expression is expected to be below detection levels in untreated cells and cells are expected to show a ligand dose-dependent increase in Cas9 expression. In contrast, the expression levels of target gene of interest are expected to be high in the untreated cells and below detection levels in ligand treated cells.

### Example 10. Ex Vivo Expansion of Stem Cells for Engraftment Using SRE Regulated Expansion Factors

To generate biocircuits for the expansion of stem cells, SREs are fused to the payload of interest, a stem cell expansion factor such as GM-CSF, IL-3, Il-6, SCF, FL, or TPO. SRE fusion constructs are cloned into retroviral vectors and transduced into freshly isolated CD34+ hematopoietic stem cells. Cells are then treated with high affinity ligand of the SRE and the stem cell expansion factor expression is measured using western blotting. An increase in expansion factor protein level indicates that the ligand is able to stabilize the construct thereby allowing its expression. Growth of the transduced hematopoietic stem cells over time is also monitored to test the functionality of the construct.

### Example 11. Regulation of Microbiome Using SREs or DDs

Microorganisms, e.g. bacteria used in microbiome therapy may be programmed to die at a specific time, after the delivery of gene or genes, and/or after the host has experienced the therapeutic effect. Standard molecular cloning techniques are used to generate a fusion of the signal response element (SRE), e.g., a destabilization domain (DD) and the payload of interest (POI), a bacterial toxin gene into an appropriate bacterial vector, generating a SRE-toxin construct. To facilitate monitoring of in vivo colonization by transformed bacteria, a detectable label, luciferase is also appended to the construct. Bacteria are transformed with the SRE-POI construct following standard transformation protocol and successfully transformed clones are sequence verified.

Colony forming unit (CFU) cell viability assays are used to measure ligand dependent protein stabilization and functionality of the SRE-toxin construct. Two overnight cultures are grown under survival conditions i.e. in the absence of ligand, following which a high affinity ligand of the SRE is added to one plate and the other plate is treated with an appropriate vehicle control. Samples are collected every two hours, serially diluted and spotted onto agar plates with appropriate survival signals. CFU and survival ratios are calculated.

To test functionality of SRE-toxin construct in vivo, mice are administered bacteria expressing SRE-toxin by oral gavage for up to 1 week. After successful colonization of the bacteria has been confirmed using bioluminescence imaging, treatment with the ligand of the SRE begins. Test group mice are injected with various concentrations of the ligand, while a control group is injected with the appropriate vehicle control. Ligand dependent expression of the toxin in the test group is expected to result in the death of colonized bacteria as measured by the loss of bioluminescence following ligand injection.

### Example 12. Regulatable Expression of Biomolecules in the Liver

The liver may be exploited as a biofactory for the production of hepatic and non-hepatic proteins such as coagulation factors, insulin, growth hormones, cytokines, and enzymes. To generate regulatable biofactory, liver tropic adeno-associated virus vectors (rAAVs) such as rAAV2 and rAAV8 are engineered with SRE fused to payload of interest e.g. immunomodulatory cytokine, IL10. rAAV vector formulations are injected into mice via the tail vein. The number of injections and the dose of rAAV formulation delivered is optimized to the payload of interest. A few days after the delivery of the rAAV, mice are injected with the ligand specific to the SRE or appropriate vehicle control. Serum is isolated from the mice and IL10 levels are measured. IL10 levels are expected to be high in ligand treated mice and below detection in in vehicle control treated mice.

### Example 13. Regulation of Ammonia Metabolizing Enzymes by SRE Based-Ammonia Biosensors

Ammonia is a key nitrogen source and a metabolic intermediate. Yet, excess ammonia can be detrimental to cell growth, especially in artificial growth environments such as bioreactors. SREs that utilize ammonia as the ligand and contain payloads that are capable of metabolizing ammonia may be useful in mitigating the negative consequences of ammonia. To engineer constructs that display ammonia dependent stability, a candidate ammonia binding domain is selected and a screened as described in Example 1 to identify a binding domain with the desired characteristics of a destabilizing domain. An ammonia metabolizing enzyme such as Carbamoyl Phosphate Synthetase 1 is selected as the Payload of interest (POI). Next, standard molecular cloning techniques are used to generate a fusion of the signal response element (SRE), e.g., ammonia sensitive-destabilization domain and the POI. Constructs are packaged into lentiviral vectors and NIH3T3 cells are transfected with lentiviral vectors. Transfected cells are treated with ammonium and the POI expression is measured using western blotting. Changes in intracellular ammonia and related metabolites is measure using mass spectrophotometry. An increase in POI protein levels in the and a concomitant increase in intracellular levels of ammonia metabolism products indicates that ammonia is able to stabilize the construct thereby allowing its expression.

### Example 14. Regulation of Protein Expression in Transgenic Animals (Mice)

Standard molecular cloning techniques are used to generate a fusion of the signal response element (SRE), e.g., a destabilization domain (DD) and the payload of interest (POI) into a retroviral plasmid, generating a SRE-POI construct. The SRE-POI constructs are then microinjected into fertilized embryos and transplanted into pseudo pregnant female mice. Resulting offspring are genotyped to identify mice carrying transgene and mice positive for the transgene. To test the expression level of the SRE-POI transgene, mice are dosed with the ligand and POI protein stabilization is analyzed. Functional effects of POI stabilization dependent on the ligand treatment are also evaluated.

### Example 15. DD Regulated IL15

To test ligand dependent IL15 production, 1 million HEK-293T cells were plated in a 6-well plate in growth media containing DMEM and 10% FBS and incubated overnight at 37° C. at 5% CO2. Cells were then transfected with 100 ng of OT-IL15-001 (constitutive) or OT-IL15-002 (ecDHFR-IL15) using Lipofectamine 2000 and incubated for 48 hrs. Following the incubation, media was exchanged for growth medium containing 10 μM Trimethoprim or vehicle control and further incubated for 24 hrs. Media was collected and the undiluted media samples or media samples diluted 4, 16, 256, 1024, 4096 or 16384-fold were tested using human IL-15 ELISA. Average IL15 ELISA readings are presented in Table 13.

TABLE 13
DD-IL15 induction

| Media dilution | | 10 μM |
|---|---|---|
| (fold) | Vehicle | TMP |
| 1 | 0.396 | 0.820 |
| 4 | 0.154 | 0.287 |
| 16 | 0.074 | 0.116 |
| 64 | 0.056 | 0.073 |
| 256 | 0.053 | 0.057 |
| 1024 | 0.053 | 0.048 |
| 4096 | 0.049 | 0.049 |
| 16384 | 0.050 | 0.049 |

The 64-fold, 16-fold, 4-fold diluted, and undiluted media samples showed IL15 levels greater than vehicle control, suggesting Trimethoprim dependent stabilization of IL15 at these dilutions.

### Example 16. DD Regulated CD19 Chimeric Antigen Receptor

A CD19 CAR fusion polypeptide was linked to either FKBP-DD or ecDHFR-DD and the constructs were cloned into pLVX-IRES-Puro vector. FKBP, and ecDHFR were positioned either between the CD19 scFv and the CD8αhinge (OT-CD19C-002, OT-CD19C-003), between the CD8αhinge and the transmembrane domain (OT-CD19C-004, OT-CD19C-005) or at the C terminus of the construct (OT-CD19C-006 and OT-CD19C-007). A constitutively expressed CAR construct, OT-CD19C-001 was used as a positive control.

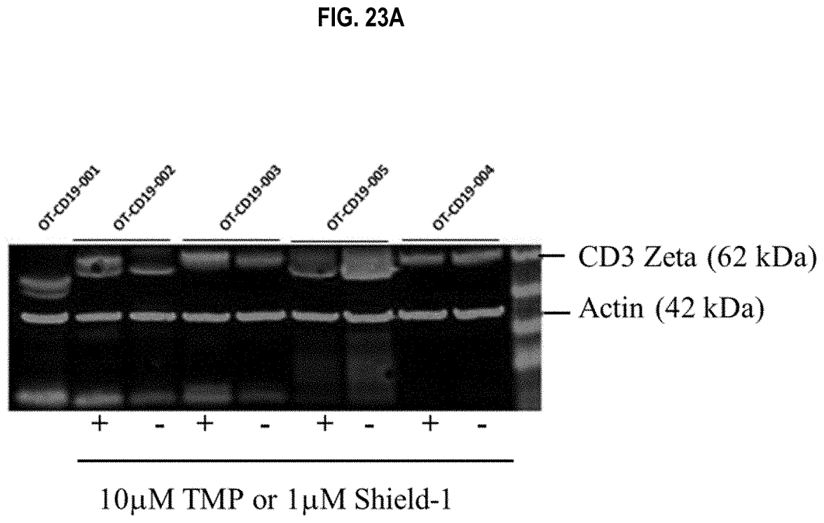

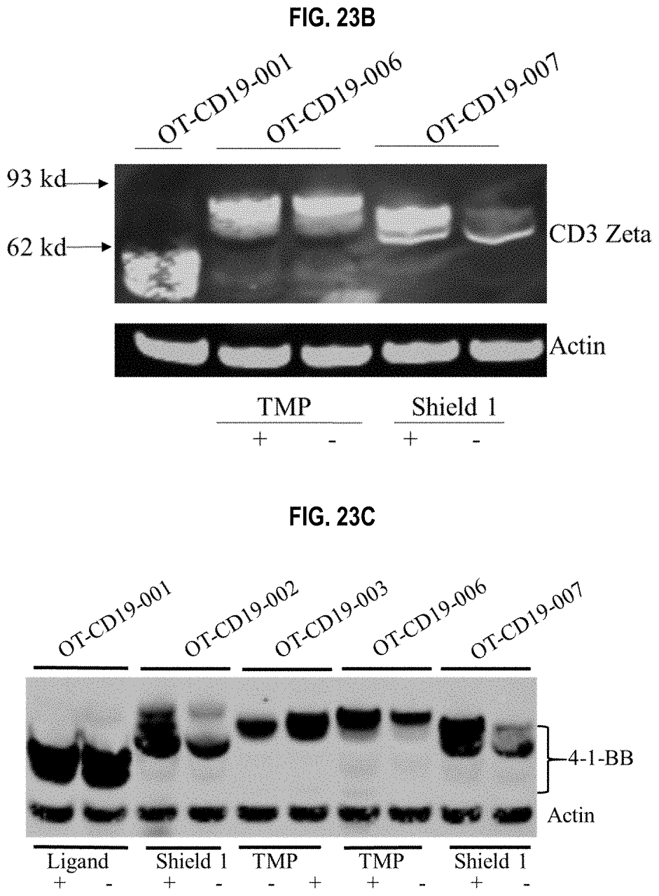

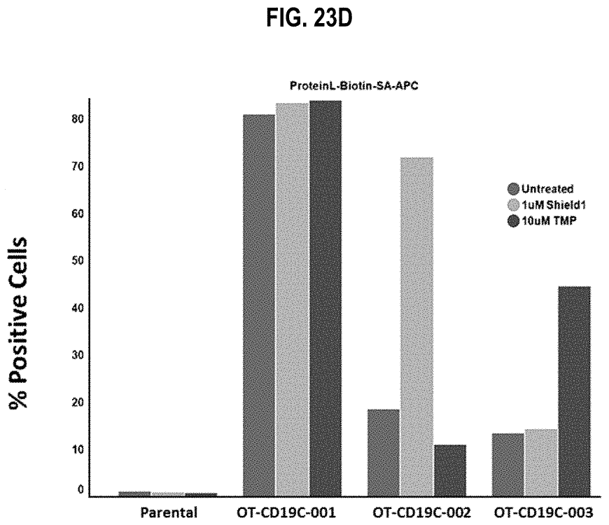

To test ligand dependent expression of DD-CD19 CAR constructs, 1 million HEK 293T cells were cultured in growth medium containing DMEM and 10% FBS and transfected with CAR constructs using Lipofectamine 2000. 48 hours after transfection, cells were treated with 1 μM or 10 μM Shield-1, 10 μM Trimethoprim, 1 μM Methotrexate, or vehicle control and incubated for 24 hours. Cells were harvested, lysed and immunoblotted for CD3 Zeta, a component of the CAR, using anti-CD247 (BD Pharmingen, Franklin Lanes, N.J.) and Alexa 555-conjugated-goat-anti mouse antibody (red) (Li-Cor, Lincoln, Nebr.). Lysates were also immunoblotted for Actin and probed with Alexa 488-conjugated secondary antibody (green) to confirm uniform protein loading in all the samples. Compared to the untreated control, OT-CD19C-002 and OT-CD19C-003 showed increased levels of CD3 Zeta in the presence of ligands, Shield-1 and TMP respectively, indicating the stabilization of the CD19 CAR (FIG. 23A). As shown in FIG. 23B, OT-CD19C-007 showed an increase in CD3 Zeta levels in the presence of Shield-1 and Methotrexate, indicating a ligand-dependent stabilization of CD19 CAR. As expected, the constutively expressed, OT-CD19C-001 showed strong expression of CD19 CAR in the absence of ligand treatment.

Lysates from cells expressing CD19 CAR constructs were also immunoblotted for 4 1-BB, a component of the CAR. As shown in FIG. 23C, OT-CD19C-003, OT-CD19C-006 and OT-CD19C-007 showed increase in 4-1BB expression levels with the treatment of corresponding ligands—TMP and Shield-1, as compared to 4-1BB levels in the absence of ligand, indicating a ligand dependent stabilization of CD19 CAR.

Surface expression of DD-CD19 CAR constructs in HEK 293T cells was measured using Fluorescence activated cell sorting (FACS) with Protein L-Biotin-Strepavidin-Allophycocyanin which binds to the kappa light chain of the CAR (ThermoFisher Scientific, Waltham, Mass.). Cells were treated with 1 μM Shield-1, 1 μM Methotrexate, 10 μM Trimethoprim or vehicle control for 24 hours and subject to FACS analysis. As shown in FIG. 23D, surface expression of OT-CD19C-002 with FKBP-DD was detected only in the presence of Shield-1, while OT-CD19C-003 with ecDHFR-DD showed surface expression only in the presence of Trimethoprim. As expected, constitutively expressed construct OT-C19C-001 showed high expression both in ligand and control vehicle treated cells.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. The present invention is further illustrated by the following non-limiting examples.

1. A regulatable human T cell engineered to express an effector module or a population of regulatable human T cells engineered to express the effector module. wherein the effector module comprises:
a first component and a second component, wherein said first component is a stimulus response element (SRE) comprising an E. coli DHFR (ecDHFR) destabilizing domain (DD), wherein the ecDHFR DD comprises the mutations (R12Y, Y100I) or (R12H, El29K), wherein said second component is a chimeric antigen receptor or a cytokine, and wherein said effector module is responsive to at least one stimulus.

2. The regulatable human T cell or population of human T cells of claim 1, wherein the chimeric antigen receptor recognizes a CD19 antigen.

3. The regulatable human T cell or population of human T cells of claim 1, wherein the cytokine is IL12 or IL15.

4. The regulatable human T cell or population of human T cells of claim 1, wherein the ecDHFR DD comprises SEQ ID NO: 5 or a sequence at least 80% sequence identity, at least 85% sequence identity, at least 90% sequence identity or at least 95% sequence identity to SEQ ID NO: 5.

5. The regulatable human T cell or population of human T cells of claim 1, wherein the ecDHFR DD comprises SEQ ID NO: 34 or a sequence at least 80% sequence identity, at least 85% sequence identity, at least 90% sequence identity or at least 95% sequence identity to SEQ ID NO: 34.

6. The regulatable human T cell or population of human T cells of claim 2, wherein the effector module comprises SEQ ID NO: 37 or SEQ ID NO: 40.

7. The regulatable human T cell or population of regulatable human T cells of claim 1, wherein the T cells are primary T cells.

8. The regulatable human T cell or population of regulatable human T cells of claim 1, wherein the T cell is selected from the group consisting of cytotoxic T cells, helper T cells, memory T cells, regulatory T cells, tissue infiltrating-T cells and combinations thereof.

9. The regulatable human T cell or population of regulatable human T cells of claim 1, wherein the human T cell or T cell population is obtained from a subject suffering from, being treated for, diagnosed with, or suspected of having a disorder selected from the group consisting of an immune disorder (including autoimmune disorders), a hyperproliferative condition, an infectious disease, a non-infectious disease, and graft vs. host disease.

10. The regulatable human T cell or population of regulatable human T cells of claim 1, wherein the stimulus is Methotrexate (MTX), or Trimethoprim (TMP).

11. A pharmaceutical composition comprising the regulatable human T cell or T cell population of regulatable human T cells of claim 1.

12. A method for treating a subject in need of T cell therapy comprising administering to the subject in need of T cell therapy the regulatable human T cell or T cell population of claim 1.

13. The method of claim 12, wherein the treatment comprises adoptive immunotherapy.

14. The method of claim 12, further comprising expanding the regulatable human T cell or T cell population prior to administration to the subject.


SEQUENCE LISTING (52 sequences)

SEQ ID NO: 1 - protein, 20 aa, synthetic construct
MYRMQLLSCIALSLALVTNS

SEQ ID NO: 2 - protein, 6 aa, synthetic construct
EFSTEF

SEQ ID NO: 3 - protein, 133 aa, synthetic construct [sequence omitted]

SEQ ID NO: 4 - protein, 107 aa, synthetic construct
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 5 - protein, 158 aa, synthetic construct
ISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 6 - protein, 268 aa, synthetic construct
MYRMQLLSCIALSLALVTNSEFSTEFGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEMHAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 319 aa, synthetic construct
MYRMQLLSCIALSLALVTNSEFSTEFISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRMHAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 22 aa, synthetic construct
MCHQQLVISWFSLVFLASPLVA

SEQ ID NO: 10 - protein, 5 aa, synthetic construct
GGSGG

SEQ ID NO: 11 - protein, 15 aa, synthetic construct
GGGGSGGGGSGGGGS

SEQ ID NO: 12 - protein, 9 aa, synthetic construct
SARNRQKRS

SEQ ID NO: 13 - protein, 8 aa, synthetic construct
ARNRQKRS

SEQ ID NO: 14 - protein, 12 aa, synthetic construct
ESRRVRRNKRSK

SEQ ID NO: 15 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 16 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 17 - protein, 107 aa, synthetic construct
GVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 18 - protein, 652 aa, synthetic construct
MCHQQLVISWFSLVFLASPLVAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGGSGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - protein, 653 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEGGSGGMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 20 - protein, 656 aa, synthetic construct
MCHQQLVISWFSLVFLASPLVAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPESARNRQKRSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - protein, 655 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASARNRQKRSGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 22 - protein, 651 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGSGGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 23 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 24 - protein, 710 aa, synthetic construct
MCHQQLVISWFSLVFLASPLVAISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRESRRVRRNKRSKIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 659 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASESRRVRRNKRSKGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 26 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 134 aa, synthetic construct
MYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 303 aa, synthetic construct
MYRMQLLSCIALSLALVTNSEFSTEFISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 29 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 31 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 42 aa, synthetic construct
KRGRKKLLYIFKQPFMRPVQTTQEEDGCSCRFPEEEEGGCEL

SEQ ID NO: 33 - protein, 21 aa, synthetic construct
MALPVTALLLPLALLLHAARP

SEQ ID NO: 34 - protein, 158 aa, synthetic construct
ISLIAALAVDHVIGMENAMPWNLPADLAWFKRNTLNKPVIMGRHTWESIGRPLPGRKNIILSSQPGTDDRVTWVKSVDEAIAACGDVPEIMVIGGGRVYEQFLPKAQKLYLTHIDAEVEGDTHFPDYKPDDWESVFSEFHDADAQNSHSYCFEILERR

SEQ ID NO: 35 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 593 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 644 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 593 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 644 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 649 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGGISLIAALAVDHVIGMENAMPWNLPADLAWFKRNTLNKPVIMGRHTWESIGRPLPGRKNIILSSQPGTDDRVTWVKSVDEAIAACGDVPEIMVIGGGRVYEQFLPKAQKLYLTHIDAEVEGDTHFPDYKPDDWESVFSEFHDADAQNSHSYCFEILERR

SEQ ID NO: 41 - protein, 598 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGSGGGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 42 - protein, 311 aa, synthetic construct
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 43 - protein, 550 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 44 - protein, 108 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPE

SEQ ID NO: 45 - protein, 108 aa, synthetic construct; identical to SEQ ID NO: 44

SEQ ID NO: 46 - protein, 159 aa, synthetic construct
MISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 47 - protein, 311 aa, synthetic construct; identical to SEQ ID NO: 42

SEQ ID NO: 48 - protein, 660 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPEEFMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 49 - protein, 420 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKPESGTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 50 - protein, 471 aa, synthetic construct
MISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERRSGTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 51 - protein, 420 aa, synthetic construct
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQSGGVQVETISPGDGRTFPKRGQTCVVHYTGMLGDGKKVDSSRDRNKPFKFMLGKQEVIRGWEEGVAQMSVGQGAKLTISPDYAYGATGHPGIIPPHATLVFDVELLELE

SEQ ID NO: 52 - protein, 471 aa, synthetic construct
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQSGISLIAALAVDYVIGMENAMPWNLPADLAWFKRNTLNKPVIMGRHTWESIGRPLPGRKNIILSSQPGTDDRVTWVKSVDEAIAACGDVPEIMVIGGGRVIEQFLPKAQKLYLTHIDAEVEGDTHFPDYEPDDWESVFSEFHDADAQNSHSYCFEILERR